(19) United States
(12) Patent Application Publication
(10) Pub. No.: US 2005/0170351 A1
(43) Pub. Date: Aug. 4, 2005

(54) Materials and methods relating to cancer diagnosis

(76) Inventors: Patrick Tan, Singapore (SG); Yu Kun, Singapore (SG); Amit Aggarwal, Singapore (SG); Chia Huey Ooi, Skudai (MY)

Correspondence Address: HARNESS, DICKEY, & PIERCE, P.L.C, 7700 BONHOMME, STE 400, ST. LOUIS, MO, 63105, US

(21) Appl. No.: 10/505,626

(22) PCT Filed: Feb. 20, 2003
(86) PCT No.: PCT/GB03/00755

(30) Foreign Application Priority Data
Feb. 20, 2002 (GB) 0203998.0
May 2, 2002 (JP) 2002-130927

(51) Int. Cl. C12Q 1/68; G01N 33/574

(52) U.S. Cl. 435/6; 435/7.23

(57) ABSTRACT

The invention provides a number of genetic identifiers (genesets) which may be used as diagnostic tools to determine the presence or risk of breast cancer in a patient. The invention also provides genesets which may be used to classify a breast tumour cell as to its molecular subgroup. Each of the identified genesets may be used to product customised specific nucleic acid microarrays for use in diagnosis and classification of breast tumour cells.

The present invention concerns materials and methods for diagnosing cancer, especially breast cancer. Particularly, but not exclusively, the invention relates to methods and kits for diagnosing the presence or risk of breast cancer using genetic identifiers.

Carcinoma of the breast is one of the leading causes of death and major illness amongst female populations worldwide. Despite rapid advances in understanding the molecular and genetic events that underlie breast carcinogenesis and the introduction of clinical screening programs, morbidity and mortality due to this disease unfortunately still remains at an unacceptably high level. Indeed, for many parts of the world, breast cancer remains one of the fastest growing cancers in local female populations (Chia et al., 2000). One major challenge in the diagnosis and treatment of breast cancer is its clinical and molecular heterogeneity. Individual breast cancers can exhibit tremendous variations in clinical presentation, disease aggressiveness, and treatment response (Tavassoli and Schitt, 1992), suggesting that this clinical entity may actually represent a conglomerate of many different and distinct cancer subtypes. In addition to variations in clinical behaviour, breast cancer can also display strikingly distinct patterns of incidence in different regional and ethnic populations. For example, in Caucasian populations, the majority of breast cancers occurs in post-menopausal women at a mean and median age of 60 and 61 respectively (Giuliano, 1998). In contrast, studies in Asian populations show a bi-modal age of incidence pattern beginning at age 40 (Chia et al., 2000, see discussion). Thus, one outstanding question in tumour biology is to explain these regional and ethnic differences on the basis of genetic or environmental factors, and to ascertain if research findings obtained using Caucasian populations can be clinically translated to other ethnic populations as well.

Expression profiling using DNA microarrays has recently proved to be an extremely powerful and versatile approach towards the investigation of multiple aspects of tumour biology. Previous reports using microarrays on breast cancers have focused on the identification of novel tumour subtypes, or on the identification of genes that are differentially expressed between known cancer subgroups (Perou et al., 2000, Gruvberger et al., 2001, Hedenfalk et al., 2001). However, because these studies have primarily focused on samples obtained primarily from Caucasian populations, it is thus an open question if the findings described in these reports will also apply to breast cancers from other ethnic populations. There are also many other key issues also need to be addressed before the use of molecular profiling can become a clinical reality. For instance, there are at present almost no published reports where the expression signatures and molecular subtypes defined in one institution's study have been independently confirmed in a separate series from another centre. Such validations are obviously essential, however, as different health-care institutions are likely to differ in multiple ways which may affect the expression profile of the tumor being studied, such as in the surgical handling of tumor samples, choice of array technology platform, and patient population base. In addition, because it is usually unfeasible to sample the same tumor over an extended period of time, it is often unclear if the different subtypes defined using these approaches truly represent distinct biological entities, or if they represent a single tumor class in different stages of clinical evolution. As one example, there are currently conflicting opinions and data in the field on whether estrogen receptor negative (ER −) breast cancers represent biological entities that have directly arisen from an ER− progenitor cell type in the breast epithelia, or if they have ‘evolved’ from an originally ER+ state (Kuukasjarri et al., 1996; Parl 2000; Gruvberger et al, 2001).

To address these issues, the inventors have embarked upon a large-scale expression profiling project of breast tumours derived from Asian patients. First, using a combination of supervised and unsupervised clustering methods, they have been able to define a small set of genes which when used in combination serves as a ‘genetic identifier’ to distinguish if an unknown breast sample is either normal or malignant in a patient of ethnic Chinese descent. The use of such ‘genetic identifiers’ is of considerable use in the development of molecular diagnostic assays for specific patient populations. Second, using principal component analysis (PCA), the inventors show that the expression profiles of normal breast tissues are considerably less varied than tumour profiles. This finding supports current models of breast tumourigenesis, in which to a first approximation normal breast tissues can be thought of as a relatively constant ‘ground state’, and that the widely varying expression profiles associated with individual tumours are probably indicative of their arising from this ‘ground state’ through many different and highly distinct tumourigenic pathways.

Third, by comparing the expression profiles of a series of invasive breast cancers from Chinese patients to published reports using patient samples of primarily Caucasian origin, they found that despite several inter-study methodological differences including choice of array technology platform, many of the key gene signatures and molecular subtypes were remarkably conserved between the two patient populations, suggesting that the molecular subtypes defined using expression-based genomics are indeed highly robust. To the inventors' knowledge, this is the first cross-institution validation study of this type reported for breast cancer.

Fourth, by studying the expression profiles of a series of ductal in-situ cancers (ductal carcinoma in situ, or DCIS), they also found that DCIS tumors express many of the ‘hallmark’ subtype-specific expression signatures associated with their invasive counterparts. Since DCIS cancers currently represent the earliest non-invasive malignant lesion detectable by conventional histopathology, these results suggest that the molecular subtypes defined in these studies probably arise at a relatively early stage of tumorigenesis (ie pre-invasive) and represent distinct biological entities, rather than a single cancer class in different stages of evolution.

Besides providing a molecular framework for the temporal progression of breast cancer, the inventors' results also support the feasibility of using expression-based genomic technologies for clinical cancer diagnosis and classification across different health-care institutions.

Thus, at its most general, the present invention provides a new diagnostic assay for determining the presence or risk of cancer, particularly breast cancer, in a patient using specific genetic identifiers. Further, the inventors have determined a series of multi-gene classifiers for breast cancer.

In the first instance, the inventors have determined a set of 20 genes (a “genetic identifier”) which may be used in combination to predict if an unknown breast tissue sample is either normal or malignant.

In addition to this first geneset (which can distinguish between tumor and normal breast samples), the inventors have also determined other genesets which, can be used as genetic identifiers to classify tumour samples as to subtype. This is of great importance, not only from a research standpoint, but also to ensure the most appropriate treatment is provided.

Thus, the inventors have determined the following genesets which may be used to predict the presence of breast tumour and/or the class of tumour.
1) The geneset provided in Table 2, which when used as a combination, allows a user to predict if an unknown breast tissue sample is either normal or malignant, particularly using spotted cDNA microarrays.
2) A further set of genes (Table 4a and 4b) which when used in combination can also be used to distinguish between normal and tumour breast tissue samples. This geneset is more preferably used on expression profiles obtained using a commercially available technology platform such as genechips, e.g. Affymetrix U133A Genechips, but can also be utilized employing the spotted cDNA microarray technology described in 1).
3) A set of genes (Table 5a) which when used in combination can predict the Estrogen Receptor status of a confirmed breast tumour sample. A second set of genes (Table 5b) which when used in combination can predict the ERBB2 status of a confirmed breast tumour sample.
4) A set of genes (Table 6) which when used in combination can be used to predict the “molecular subtype” of a breast tumour sample according to the following 5 categories: Luminal, Basal, ERBB2, Normal-like, and ER-negative subtype II. In this embodiment of the present invention, the inventors have used two different types of classification algorithms, namely, (1) one-vs-all (OVA) support vector machines (SVM); and (2) genetic algorithm (GA/maximum likelihood discriminant (MLHD) analysis. Different sets of genes are optimally used depending upon the type of classification algorithm used. Thus, distinct sets of genes are described below for each part.
5) A set of genes (Table 7) which when used in combination can be used to predict luminal subclass in Asian breast cancer patients. The inventors have determined that breast tumours of the “luminal” variety can be “split” into two distinct subtypes Luminal A and Luminal D which are clinically relevant. The genetic identifier (Table 7) is therefore preferably used after the tumour has been formally recognised as “luminal” in nature. This of course, can be achieved using the multi-class predictor of Table 6. The Luminal D tumours are associated with certain expression signatures that are also found highly aggressive non-Luminal tumours, e.g. ERBB2 and Basal. This supports the clinical importance of knowing the tumour subtype.

The determination of specific genesets (genetic identifiers) allows tissue samples to be classified (e.g. tumour v normal) according to the expression pattern of those genes in the tissue. For example, in the first genetic identifier (tumor vs normal) the inventors have determined 10 genes that are usually up-regulated in tumour cells relative to normal cells and 10 genes that are usually down-regulated in tumour cells relative to normal cells. By studying the expression pattern of these particular genetic identifiers, i.e. the composite levels of expression products of these genes in a test sample, it is possible to classify the sample as malignant or normal. Thus, the expression products are able to provide an expression profile or “fingerprint” that can serve to distinguish between normal and malignant cells.

In a first aspect of the present invention, there is provided a method of creating a nucleic acid expression profile for a breast tumour cell comprising the steps of
(a) isolating expression products from said breast tumour cell and a normal breast cell;
(b) identifying the expression profile of a plurality of genes selected from Table 2; for both the tumour and normal cell;
(c) comparing the expression profile of the tumour cell and the normal cell; and
(d) determining a nucleic acid expression profile characteristic of a breast tumour cell.

For the purposes of diagnosis, it is important to obtain an expression profile that is characteristic of a tumour cell, i.e. distinct from the expression profile of the equivalent normal cell. The method according to the first aspect determines the expression profile of a plurality of genes identified by the inventors to be a “genetic identifier” of breast tumour cells (see Table 2).

The expression profile of the individual genes that comprise the genetic identifier will differ slightly between independent samples. However, the inventors have realised that the expression profile of these particular genes that comprise the genetic identifier when used in combination provide a characteristic pattern of expression (expression profile) in a tumour cell that is recognisably different from that in a normal cell.

By creating a number of expression profiles of the genetic identifier from a number of known tumour or normal samples, it is possible to create a library of profiles for both normal and tumour samples. The greater the number of expression profiles, the easier it is to create a reliable characteristic expression profile standard (i.e. including statistical variation) that can be used as a control in a diagnostic assay. Thus, a standard profile may be one that is devised from a plurality of individual expression profiles and devised within statistical variation to represent either the tumour or normal cell profile.

Thus, the method according to the first aspect of the invention comprises the steps of
(a) isolating expression products from a breast tumour cell; contacting said expression products with a plurality of binding members capable of specifically and independently binding to expression products of a plurality of genes selected from Table 2, so as to create a first expression profile of a tumour-cell;
(b) isolating expression products from a normal breast cell; contacting said expression products with the plurality of binding members used in step (a), so as to create a comparable second expression profile of a normal breast cell;
(c) comparing the first and second expression profiles to determine an expression profile characteristic of a breast tumour cell.

The expression products are preferably mRNA, or cDNA made from said mRNA. Alternatively, the expression product could be an expressed polypeptide. Identification of the expression profile is preferably carried out using binding members capable of specifically identifying the expression products of genes identified in Table 2. For example, if the expression products are cDNA then the binding members will be nucleic acid probes capable of specifically hybridising to the cDNA.

Preferably, either the expression product or the binding member will be labelled so that binding of the two components can be detected. The label is preferably chosen so as to be able to detect the relative levels/quantity and/or absolute levels/quantity of the expressed product so as to determine the expression profile based on the up-regulation or down-regulation of the individual genes that comprise the genetic identifiers. In other words, it is preferable that the binding members are capable of not only detecting the presence of an expression product but its relative abundance (i.e. the amount of product available).

The determination of the nucleic acid expression profile may be computerised and may be carried out within certain previously set parameters, to avoid false positives and false negatives.

The computer may then be able to provide an expression profile standard characteristic of a normal breast cell and a malignant breast cell as discussed above. The determined expression profiles may then be used to classify breast tissue samples as normal or malignant as a way of diagnosis.

Thus, in a second aspect of the invention, there is provided an expression profile database comprising a plurality of gene expression profiles of both normal and malignant breast cells where the genes are selected from Table 2; retrievably held on a data carrier. Preferably, the expression profiles making up the database are produced by the method according to the first aspect.

With the knowledge of the particular genetic identifiers, it is possible to devise many methods for determining the expression pattern or profile of the genes in a particular test sample of cells. For example, the expressed nucleic acid (RNA, mRNA) can be isolated from the cells using standard molecular biological techniques. The expressed nucleic acid sequences corresponding to the gene members of the genetic identifiers given in Table 2 can then be amplified using nucleic acid primers specific for the expressed sequences in a PCR. If the isolated expressed nucleic acid is mRNA, this can be converted into cDNA for the PCR reaction using standard methods.

The primers may conveniently introduce a label into the amplified nucleic acid so that it may be identified. Ideally, the label is able to indicate the relative quantity or proportion of nucleic acid sequences present after the amplification event, reflecting the relative quantity or proportion present in the original test sample. For example, if the label is fluorescent or radioactive, the intensity of the signal will indicate the relative quantity/proportion or even the absolute quantity, of the expressed sequences. The relative quantities or proportions of the expression products of each of the genetic identifiers will establish a particular expression profile for the test sample. By comparing this profile with known profiles or standard expression profiles, it is possible to determine whether the test sample was from normal breast tissue or malignant breast tissue.

Alternatively, the expression pattern or profile can be determined using binding members capable of binding to the expression products of the genetic identifiers, e.g. mRNA, corresponding cDNA or expressed polypeptide. By labelling either the expression product or the binding member it is possible to identify the relative quantities or proportions of the expression products and determine the expression profile of the genetic identifiers. In this way the sample can be classified as normal or malignant by comparison of the expression profile with known profiles or standards. The binding members may be complementary nucleic acid sequences or specific antibodies. Microarray assays using such binding members are discussed in more detail below.

In a third aspect of the present invention, there is provided a method for determining the presence or risk of breast cancer in a patient comprising the steps of
(a) obtaining expression products from breast tissue cells obtained from a patient suspected of having or at risk of having breast cancer;
(b) contacting said expression products with one or more binding members capable of detecting the presence of an expression product corresponding to one or more genes identified in Table 2; and
(c) determining the presence or risk of breast cancer in said patient based on the binding profile of the expression products from the breast tissue cells to the one or more binding members.

The patient is preferably a woman of Asian descent, e.g. ethnic Chinese descent.

The step of determining the presence or risk of breast cancer may be carried out by a computer which is able to compare the binding profile of the expression products from the breast tissue cells under test with a database of other previously obtained profiles and/or a previously determined “standard” profile which is characteristic of the presence or risk of the tumour. The computer may be programmed to report the statistical similarity between the profile under test and the standard profiles so that a diagnosis may be made.

As mentioned above, the present inventors have identified several key genes which have a different expression pattern in tumour cells as opposed to normal cells of the breast. Collectively, these genes comprise a ‘genetic identifier’. The inventors have shown (see below) that the combinatorial expression pattern of the genes belonging to the “genetic identifier” serves to distinguish between normal and tumour cells. Thus, by detecting the expression pattern of the genetic identifier in a breast tissue sample, it is possible to predict the state of the cell (normal or malignant) and whether that patient has or is at risk of developing breast cancer.

The genes that comprise the genetic identifier are given in Table 2. There are 20 genes shown, 10 of which are commonly highly expressed in tumour cells relative to normal cells and 10 of which commonly have decreased expression in tumour cells relative to normal cells. The differential expression of the genes was determined using tumour biopsies and normal tissue biopsies. By detecting the levels of expression products of these genes in a test sample, it is possible to classify the cells as normal or malignant based on the expression profile produced, i.e. an increase or decrease in their expression, relative to a standard pattern or profile seen in normal cells.

Thus, in a further aspect of the invention, there is provided a method of classifying a sample of breast tissue as normal or malignant, said method comprising the steps of
a) obtaining expression products from the cells of the breast tissue sample;
b) contacting said expression products with a plurality of binding members capable of specifically binding to the expression products of a plurality of genes selected from Table 2; and
c) classifying the sample as normal or malignant based on the binding profile of the expression products from the sample and the binding members.

The sample of breast tissue is preferably from a woman of Asian descent, e.g. ethnic Chinese descent.

As before, the expression product may be a transcribed nucleic acid sequence or the expressed polypeptide. The transcribed nucleic acid sequence may be RNA or mRNA. The expression product may also be cDNA produced from said mRNA.

The binding member may a complementary nucleic acid sequence which is capable of specifically binding to the transcribed nucleic acid under suitable hybridisation conditions. Typically, cDNA or oligonucleotide sequences are used.

Where the expression product is the expressed protein, the binding member is preferably an antibody, or molecule comprising an antibody binding domain, specific for said expressed polypeptide.

The binding member may be labelled for detection purposes using standard procedures known in the art. Alternatively, the expression products may be labelled following isolation from the sample under test. A preferred means of detection is using a fluorescent label which can be detected by a light meter. Alternative means of detection include electrical signalling. For example, the Motorola e-sensor system has two probes, a “capture probe” which is freely floating, and a “signalling probe” which is attached to a solid surface which doubles as an electrode surface. Both probes function as binding members to the expression product. When binding occurs, both probes are brought into close proximity with each other resulting in the creation of an electrical signal which can be detected.

As discussed above, the binding members may be oligonucleotide primers for use in a PCR (e.g. multi-plexed PCR) to specifically amplify the number of expressed products of the genetic identifiers. The products would then be analysed on a gel. However, preferably, the binding member a single nucleic acid probe or antibody fixed to a solid support. The expression products may then be passed over the solid support, thereby bringing them into contact with the binding member. The solid support may be a glass surface, e.g. a microscope slide; beads (Lynx); or fibre-optics. In the case of beads, each binding member may be fixed to an individual bead and they are then contacted with the expression products in solution.

Various methods exist in the art for determining expression profiles for particular gene sets and these can be applied to the present invention. For example, bead-based approaches (Lynx) or molecular bar-codes (Surromed) are known techniques. In these cases, each binding member is attached to a bead or “bar-code” that is individually readable and free-floating to ease contact with the expression products. The binding of the binding members to the expression products (targets) is achieved in solution, after which the tagged beads or bar-codes are passed through a device (e.g. a flow-cytometer) and read.

A further known method of determining expression profiles is instrumentation developed by Illumina, namely, fibre-optics. In this case, each binding member is attached to a specific “address” at the end of a fibre-optic cable. Binding of the expression product to the binding member may induce a fluorescent change which is readable by a device at the other end of the fibre-optic cable.

The present inventors have successfully used a nucleic acid microarray comprising a plurality of nucleic acid sequences fixed to a solid support. By passing nucleic acid sequences representing expressed genes e.g. cDNA, over the microarray, they were able to create an binding profile characteristic of the expression products from tumour cells and normal cells derived from breast tissue.

The present invention further provides a nucleic acid microarray for classifying a breast tissue sample as malignant or normal comprising a solid support housing a plurality of nucleic acid sequences, said nucleic acid sequences being capable of specifically binding to expression products of one or more genes identified in Table 2. The classification of the sample will lead to the diagnosis of breast cancer in a patient. Preferably the solid support will house nucleic acid sequences being capable of specifically and independently binding to expression products of at least 5 genes, more preferably, at least 10 genes or at least 15 genes identified in Table 2. In a most preferred embodiment, the solid support will house nucleic acid sequences being capable of specifically and independently binding to expression products of all 20 genes identified in Table 2.

Typically, high density nucleic acid sequences, usually cDNA or oligonucleotides, are fixed onto very small, discrete areas or spots of a solid support. The solid support is often a microscopic glass side or a membrane filter, coated with a substrate (or chips). The nucleic acid sequences are delivered (or printed), usually by a robotic system, onto the coated solid support and then immobilized or fixed to the support.

In a preferred embodiment, the expression products derived from the sample are labelled, typically using a fluorescent label, and then contacted with the immobilized nucleic acid sequences. Following hybridization, the fluorescent markers are detected using a detector, such as a high resolution laser scanner. In an alternative method, the expression products could be tagged with a non-fluorescent label, e.g. biotin. After hybridisation, the microarray could then be ‘stained’ with a fluorescent dye that binds/bonds to the first non-fluorescent label (e.g. fluorescently labelled strepavidin, which binds to biotin).

A binding profile indicating a pattern of gene expression (expression pattern or profile) is obtained by analysing the signal emitted from each discrete spot with digital imaging software. The pattern of gene expression of the experimental sample can then be compared with that of a control (i.e. an expression profile from a normal tissue sample) for differential analysis.

As mentioned above, the control or standard, may be one or more expression profiles previously judged to be characteristic of normal or malignant cells. These one or more expression profiles may be retrievable stored on a data carrier as part of a database. This is discussed above. However, it is also possible to introduce a control into the assay procedure. In other words, the test sample may be “spiked” with one or more “synthetic tumour” or “synthetic normal” expression products which can act as controls to be compared with the expression levels of the genetic identifiers in the test sample.

Most microarrays utilize either one or two fluorophores. For two-colour arrays, the most commonly used fluorophores are Cy3 (green channel excitation) and Cy5 (red channel excitation). The object of the microarray image analysis is to extract hybridization signals from each expression product. For one-color arrays, signals are measured as absolute intensities for a given target (essentially for arrays hybridized to a single sample). For two-colour arrays, signals are measured as ratios of two expression products, (e.g. sample and control (controls are otherwise known as a ‘reference’)) with different fluorescent labels.

The microarray in accordance with the present invention preferably comprises a plurality of discrete spots, each spot containing one or more oligonucleotides and each spot representing a different binding member for an expression product of a gene selected from Table 2. In a preferred embodiment, the microarray will contain 20 spots for each of the 20 genes provided in Table 2. Each spot will comprise a plurality of identical oligonucleotides each capable of binding to an expression product, e.g. mRNA or cDNA, of the gene of Table 2 it is representing.

In a still further aspect of the present invention, there is provided a kit for classifying a breast tissue sample as normal or malignant, said kit comprising one or more binding members capable of specifically binding to an expression product of one or more genes identified in Table 2, and a detection means.

Preferably, the one or more binding members (antibody binding domains or nucleic acid sequences e.g. oligonucleotides) in the kit are fixed to one or more solid supports e.g. a single support for microarray or fibre-optic assays, or multiple supports such as beads. The detection means is preferably a label (radioactive or dye, e.g. fluorescent) for labelling the expression products of the sample under test. The kit may also comprise means for detecting and analysing the binding profile of the expression products under test.

Alternatively, the binding members may be nucleotide primers capable of binding to the expression products of the genes identified in Table 2 such that they can be amplified in a PCR. The primers may further comprise detection means, i.e. labels that can be used to identify the amplified sequences and their abundance relative to other amplified sequences.

The kit may also comprise one or more standard expression profiles retrievably held on a data carrier for comparison with expression profiles of a test sample. The one or more standard expression profiles may be produced according to the first aspect of the present invention.

The present invention further provides a method of diagnosing the presence or risk of breast cancer in a patient of Asian descent, said method comprising
obtaining a breast tissue sample;
isolating expression products from said sample;
labelling said expression products;
contacting said labelled expression products with a plurality of binding members representing a plurality of genes selected from Table 2;
determining the presence or risk of breast cancer in said patient, based on the binding profile of said labelled expression products and the binding members.

The breast tissue sample may be obtained as excisional breast biopsies or fine-needle aspirates.

Again, the expression products are preferably mRNA or cDNA produced from said mRNA. The binding members are preferably oligonucleotides fixed to one or more solid supports in the form of a microarray or beads (see above). The binding profile is preferably analysed by a detector capable of detecting the label used to label the expression products. The determination of the presence or risk of breast cancer can be made by comparing the binding profile of the sample with that of a control e.g. standard expression profiles.

In all of the aspects described above, it is preferred to use binding members capable of specifically binding (and, in the case of nucleic acid primers, amplifying) expression products of all 20 genetic identifiers. This is because the expression levels of all 20 genes make up the expression profile specific for the cells under test. The classification of the expression profile is more reliable the greater number of gene expression levels tested. Thus, preferably expression levels of more than 5 genes selected from Table 2 are assessed, more preferably, more than 10, even more preferably, more than 15 and most preferably all 20 genes.

The genetic identifier (Table 2) mentioned above is particularly suitable for spotted cDNA microarray technology where the microarray (or other similar technology) has been created specifically for this purpose. However, the present inventors have appreciated that the present invention may be modified so that commercially available genechips may be used, rather than going to the trouble of creating one specifically containing the genes identified in Table 2. With this in mind, the inventors have identified a further genetic identifier (Table 5a or 5b) which, although it may be utilized using microarray technology described above, it may also be used on commercially available genechips, e.g. Affymetrix U133A Genechips.

Thus, the aspects of the invention described above may also be carried out using the geneset of Table 4a or 4b instead of that of Table 2 and in addition these may be used on either on commercially available genechips such as Affymetrix U133A Genechips, or using microarray technology described above.

The present inventors have also identified a further set of genes (Table 5a) which may be used to classify a breast tumour on the basis of the Estrogen Receptor (ER) status. This is clinically important as ER⁺ tumours can be treated with hormonal therapies (e.g. tamoxifen) and ER⁻ tumours are typically more aggressive and refractory to treatment.

Likewise, the present inventors have also identified a further set of genes (Table 5b) which may be used to classify a breast tumour on the basis of the ERBB2+ status. Knowing the ERBB2⁺ status of a breast tumour is also clinically important as ERBB2⁺ tumours are typically highly aggressive and carry a poor clinical prognosis. ERBB2+ tumors are also candidates for treatment with Herceptin (an anti-cancer drug).

The genesets provided in Tables 5a and 5b were determined by generating expression profiles for a set of breast tumour samples using Affymetrix U133A Genechips. A series of statistical algorithms were used to identify a set of genes that were differentially expressed in ER⁺ vs ER⁻ samples as well as ERBB2⁺ vs ERBB2⁻ samples. Accordingly, the present invention further provides genesets which may be used in methods of classifying breast tumours according to ER and ERBB2 status.

Thus, in a further aspect of the present invention, there is provided a method of classifying a breast tumour according to its ER and/or ERBB2 status comprising.
a) obtaining expression products from the tumour cells;
b) contacting said expression products with a plurality of binding members capable of specifically binding to the expression products of a plurality of genes selected from Table 5; and
c) classifying the tumour cell on the basis of ER and/or ERBB2 status based on the binding profile of the expression products from the sample and the binding members.

As with the first aspect of the present invention, the plurality of binding members are preferably nucleic acid sequences and more preferably nucleic acid sequences fixed to a solid support, for example as a nucleic acid microarray. The nucleic acid sequences may be oligonucleotide probes or cDNA sequences.

The tumour cell may be classified according to its ER and/or ERBB2 status on the basis of the expression of the genes identified in Table 5. Table 5 identifies each gene as either being upregulated (+) or down regulated (−) in an ER⁺ or ERBB2⁺ tumour. With this information, it is possible to determine whether the breast tumour cell under test is ER⁻ or ER⁺ and/or ERBB2⁺ or ERBB2⁻.

As with all aspects of the present invention, the plurality of genes selected from the determined genesets (Tables 2-7 with the exception of Table 6b) may vary in actual number. It is preferable to use at least 5 genes, more preferably at least 10 genes in order to carry out the invention. Of course, the known microarray and genechip technologies allow large numbers of binding members to be utilized. Therefore, the more preferred method would be to use binding members representing all of the genes in each geneset. However, the skilled person will appreciate that a proportion of these genes may be omitted and the method still carried out in a reliable and statistically accurate fashion. In most cases, it would be preferable to use binding members representing at least 70%, 80% or 90% of the genes in each respective geneset.

In a further aspect of the invention, there is provided a method of classifying a breast tumour cell as to its molecular subtype comprising
a) obtaining expression products from the tumour cells;
b) contacting said expression products with a plurality of binding members capable of specifically binding to the expression products of a plurality of genes selected from Table 6; and
c) classifying the tumour cell with regard to its molecular subtype based on the binding profile of the expression products from the tumour cell and the binding members.

The molecular subtypes are preferably Luminal, ERBB2, Basal, ER-type II and Normal/normal like. These sub-types are defined in the following text.

In practice, the expression profile of the tumour sample to be classified is determined using the genesets described in Table 6 (Table 6a or 6b depends on the type of classification algorithm used). Secondly, the expression profile would be compared to a database of “references” (control profiles, where each “reference” (control) profiles, where each “reference” profile corresponds to the “average” tumour belonging to that particular molecular type. In this case, rather than just having normal and tumour, or ER⁺ and ER⁻, the “reference” profiles will correspond to five distinct subtypes. Third, by using a suitable classification algorithm, the unknown tumour sample can be assigned to the specific subtype for which the expression profile finds a good reference match.

Where the plurality of binding members are selected as being capable of binding to the expression products of a plurality of genes from Table 6a, the number of binding members used will govern the reliability of the test. In other words, it is not necessary to use binding members capable of specifically and independently to all genes identified in Table 6a, but the more binding members used, the better the test. Therefore, by plurality it is meant preferably at least 50%, more preferably at least 70% and even more preferably at least 90% of the genes as mentioned above.

In a still further aspect of the invention, there is provided a method of further sub-classifying a breast tumour cell as either luminal A or luminal D subtype comprising
a) obtaining expression products from the tumour cells;
(b) contacting said expression-products with a plurality of binding members capable of specifically binding to the expression products of a plurality of genes selected from Table 7; and
c) classifying the tumour cell with regard to its molecular subtype based on the binding profile of the expression products from the tumour cell and the binding members.

Preferably, the method is carried out on expression products obtained from a breast tumour cell which has already been classified as “luminal”, e.g. using the genetic identifier of Table 6a or 6b.

With regard to the geneset provided in Table 6b, it is preferable that all of the genes in the geneset are used for classification. The reduction in the number of genes will take away the likelihood of a reliable result. This is because this geneset is selected using the genetic algorithm approach.

The inventors have provided a number of genetic identifiers (Tables 2 to 7) which can be used to diagnose and/or predict risk of breast cancer and, further, can be used to classify the type of breast cancer, particularly for women of Asian descent.

The provision of these genetic identifiers allows diagnostic tools, e.g. nucleic acid microarrays to be custom made and used to predict, diagnose or subtype tumours. Further, such diagnostic tools may be used in conjunction with a computer which is programmed to determine the expression profile obtained using the diagnostic tool (e.g. microarray) and compare it to a “standard” expression profile characteristic of normal v tumour and/or molecular subtypes depending on the particular genetic identifier used. In doing so, the computer not only provides the user with information which may be used diagnose the presence or type of a tumour in a patient, but at the same time, the computer obtains a further expression profile by which to determine the “standard” expression profile and so can update its own database.

Thus, the invention allows, for the first time, specialized chips (microarrays) to be made containing probes corresponding to the genesets identified in Tables 2 to 7. The exact physical structure of the array may vary and range from oligonucleotide probes attached to a 2-dimensional solid substrate to free-floating probes which have been individually “tagged” with a unique label, e.g. “bar code”.

A database corresponding to the various biological classifications (e.g. normal, tumour, molecular subtype etc.) may be created which will consist of the expression profiles of various breast tissues as determined by the specialized microarrays. The database may then be processed and analysed such that it will eventually contain (i) the numerical data corresponding to each expression profile in the database, (ii) a “standard” profile which functions as the canonical profile for that particular classification; and (iii) data representing the observed statistical variation of the individual profiles to the “standard” profile.

In practice, to evaluate a patient's sample, the expression products of that patient's breast cells (obtained via excisional biopsy or find needle aspirate) will first be isolated, and the expression profile of that cell determined using the specialized microarray. To classify the patient's sample, the expression profile of the patient's sample will be queried against the database described above. Querying can be done in a direct or indirect manner. The “direct” manner is where the patient's expression profile is directly compared to other individual expression profiles in the database to determined which profile (and hence which classification) delivers the best match. Alternatively, the querying may be done more “indirectly”, for example, the patient expression profile could be compared against simply the “standard” profile in the database. The advantage of the indirect approach is that the “standard” profiles, because they represent the aggregate of many individual profiles, will be much less data intensive and may be stored on a relatively inexpensive computer system which may then form part of the kit (i.e. in association with the microarrays) in accordance with the present invention. In the direct approach, it is likely that the data carrier will be of a much larger scale (e.g. a computer server) as many individual profiles will have to be stored.

By comparing the patient expression profile to the standard profile (indirect approach) and the pre-determined statistical variation in the population, it will also be possible to deliver a “confidence value” as to how closely the patient expression profile matches the “standard” canonical profile. This value will provide the clinician with valuable information on the trustworthiness of the classification, and, for example, whether or not the analysis should be repeated.

As mentioned above, it is also possible to store the patient expression profiles on the database, and these may be used at any time to update the database.

Aspects and embodiments of the present invention will now be illustrated, by way of example, with reference to the accompanying figures. Further aspects and embodiments will be apparent to those skilled in the art. All documents mentioned in this text are incorporated herein by reference

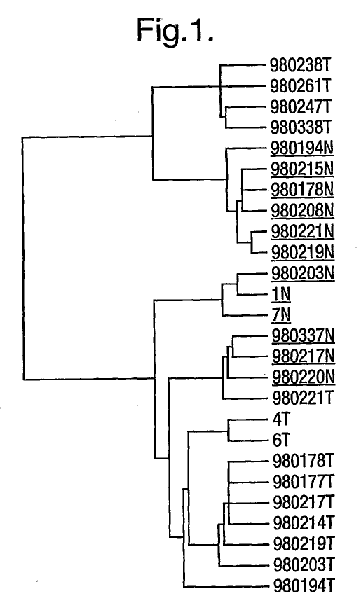

FIG. 1: Unsupervised Partitioning of Normal and Tumour Breast Samples. Individual expression profiles were subjected to standard data selection filters (see text), and the resultant data matrix, comprising approximately 800 array targets, was sorted using hierarchical clustering. Normal samples (‘xxxN’) are underlined, while tumour samples (‘xxxT’) are not. Numbers represent the NCC Tissue Repository numbers associated with each sample. The dendogram branches illustrate the extent of similarity between the biological samples. Normal and Tumour samples segregate independently, but only at secondary levels of the dendogram. Minor variations on the data filters used to select this data set also yielded highly similar dendograms (P. Tan, unpublished observations)

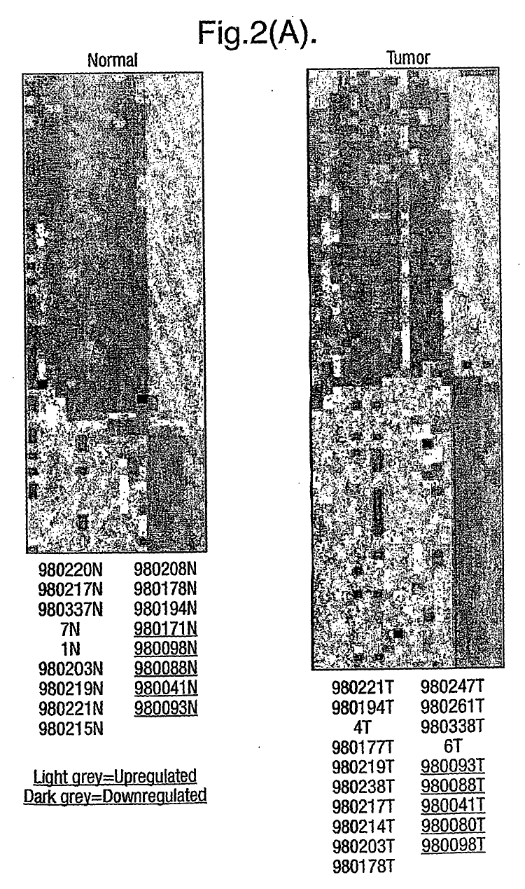

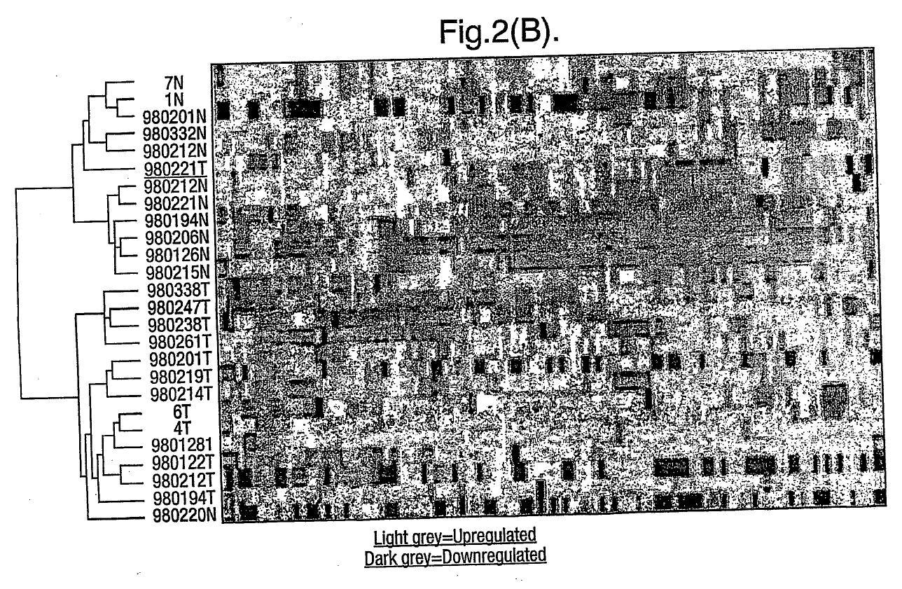

FIG. 2: Improvement of Normal and Tumour Sample Partitioning Using Combined Outlier Genesets (COG). (A) Independent outlier genesets for normal (left) and tumour (right) samples were defined. Each clustergram consists of a matrix of array targets (rows) by biological samples (columns), and light grey represents upregulation, while dark grey represents downregulation (see Materials and Methods for selection criteria). The outlier geneset for normal samples consists of 60 genes, while the outlier geneset for tumour samples consists of 75 genes. Specific normal and tumour samples used in the establishment of the outlier genesets are listed below each clustergram. Underlined sample numbers indicate reciprocal hybridizations, where the tumour/normal sample was labelled using Cy5 and the reference sample Cy3. (B) Partitioning of normal and tumour samples using the COG. The 108 unique array targets comprising the COG were used to segregate the tumour and normal samples from FIG. 1 using standard hierarchical clustering. In contrast to FIG. 1, division of the normal (xxxN) and tumour (xxxT) samples is now observed as a primary class division, with 2 misclassifications.

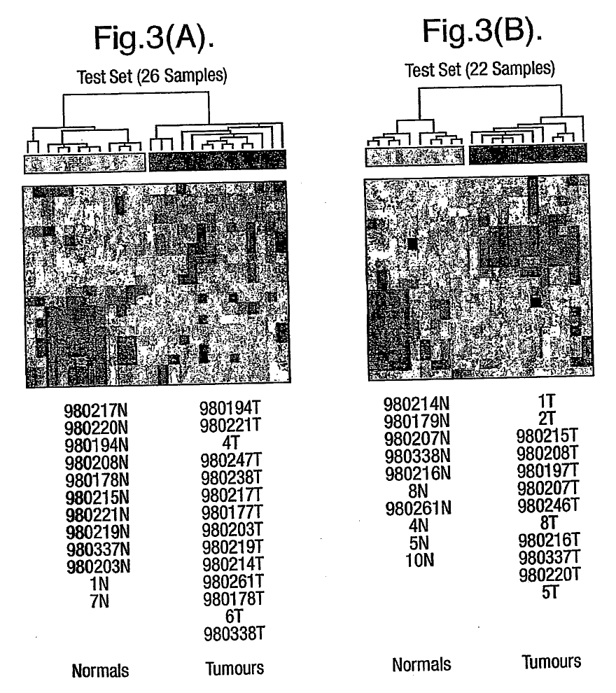

FIG. 3: Partitioning of Normal and Tumour Samples using a Minimal 20-Element Genetic Identifier. The 20 array targets from the COG (Table 2) that were most highly correlated to the tumour/normal class distinction were used to segregate (A) the training set from FIGS. 1 and 2b, and (B) a naïve test set of 10 normals and 11 tumours. In both cases, accurate segregation of normal and tumour samples at the level of the primary class division can be observed.

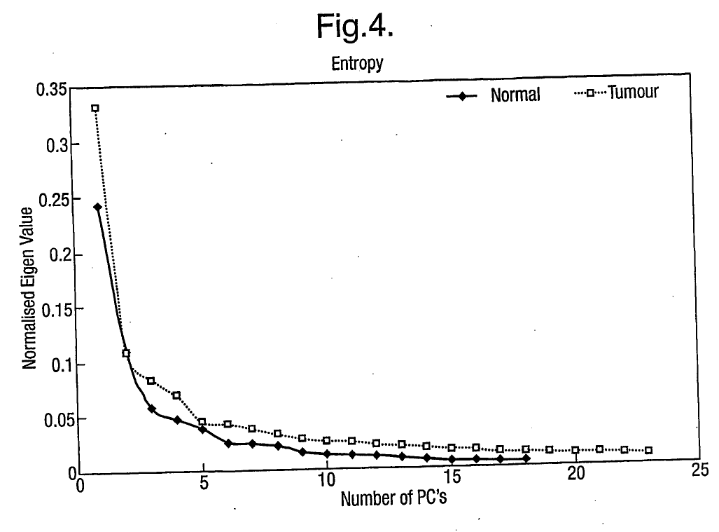

FIG. 4: Comparison of expression profile variation in normal and tumour samples. Independent normal and tumour datasets were established using the combined samples of FIGS. 3a and 3b (total=48 samples). Using PCA, the entire gene expression matrix of approximately 8000 array targets in these datasets were reduced to basic principal components. The extent of variance of each component normalized to the 1^{st} component (normalized eigenvalue) is depicted on the y-axis, and the principal component number on the x-axis, beginning with the 2^{nd} component (since the first component of each set is 1). To observe the rate of ‘decay’ of information, the components for each dataset are depicted in decreasing order of variance. Normal samples consistently exhibit a lower information decay rate across their components compared with tumours.

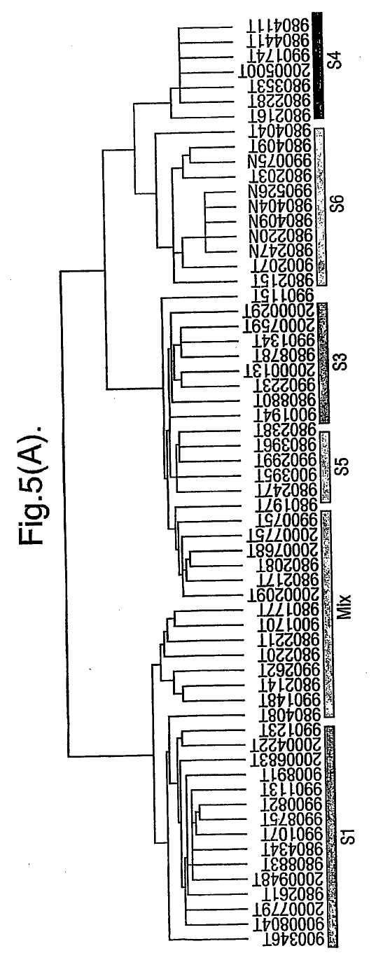

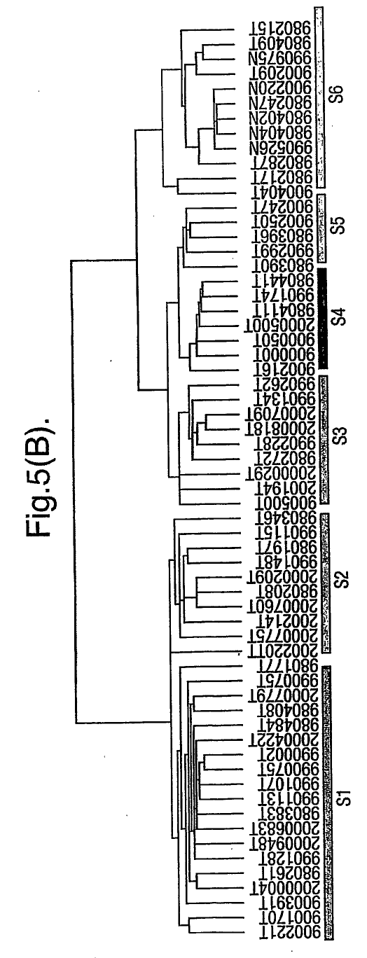

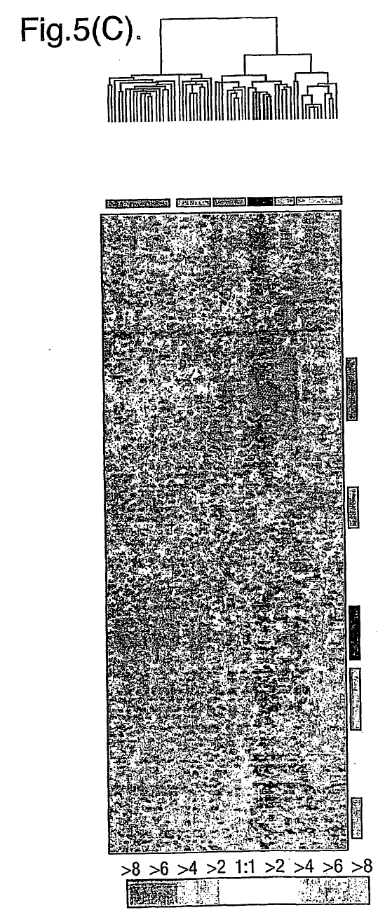

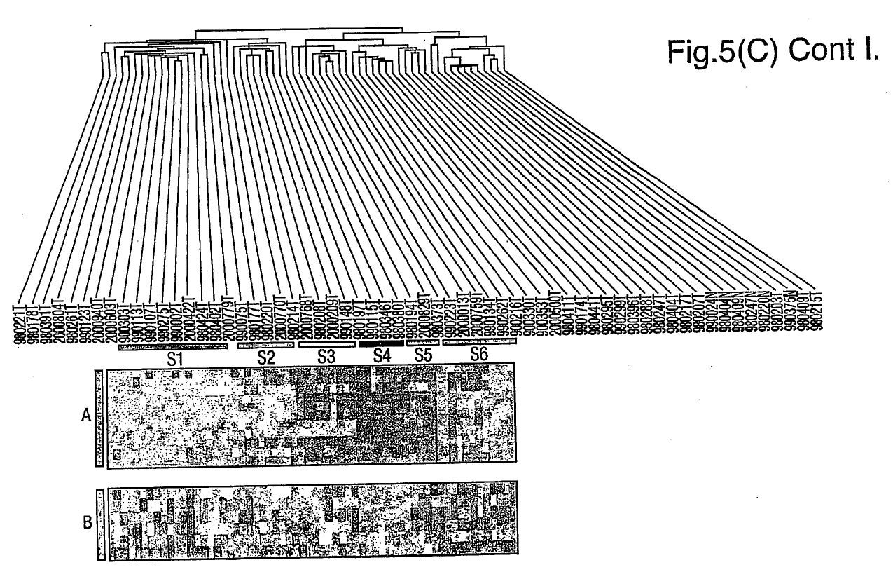

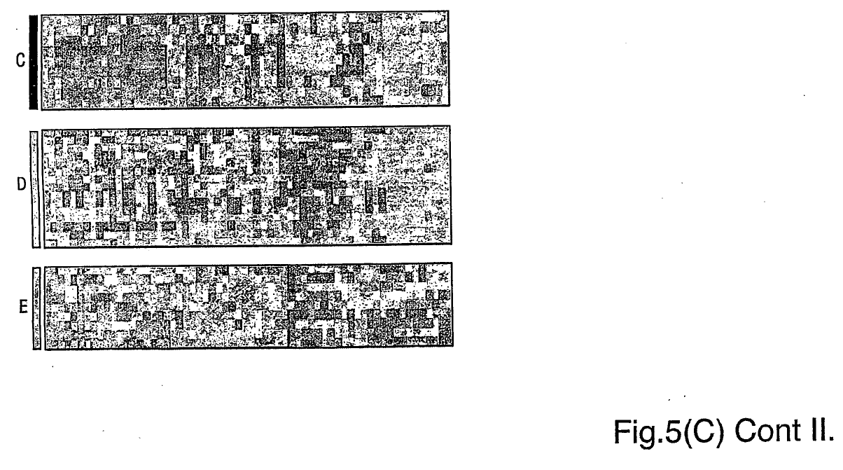

FIG. 5: Gene expression patterns of 62 samples including 56 carcinomas and 6 normal tissues, analyzed by hierarchical clustering using different gene sets. Samples were divided into 6 subtypes based on differences in gene expression (legend), and are: Luminal, (S1); ERBB2+/ER+ (S2, ERBB2+/er− (S3), Basal-like (S4), ER negative subtype II (S5), and Normal/Normal-like (S6)

(a) Unsupervised hierarchical clustering using a dataset of 1796 genes. The gray underline indicates a cluster which contains a mixture of Luminal and ERBB2+/ER+ samples. (b) Semi-supervised hierarchical clustering using the ‘common intrinsic gene set’ (CIS, 292 genes). (c) The full cluster diagram using the CIS. Shaded bars to the right of the clustergram represent gene clusters A-E (Table 3), and are (A) Luminal epithelial genes with ER. (B) ‘Novel’ genes. (C) Basal epithelial genes. (D) Normal breast-like genes. (E) ERBB2-related genes.

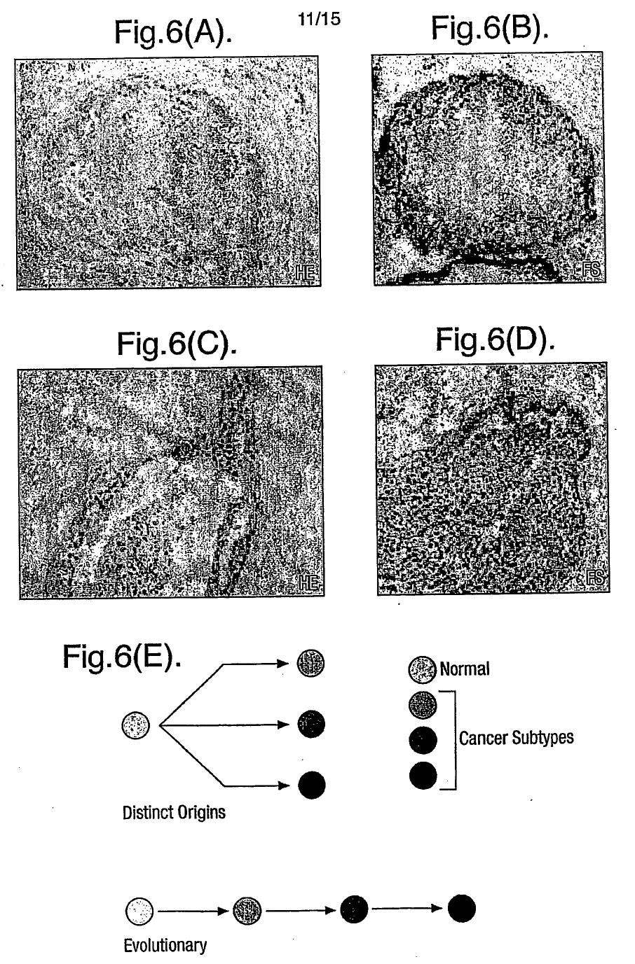

FIG. 6(a)-(d) Representative Examples of DCIS Samples Used in this Study. Two samples are shown (a)/(b), and (c)/(d) The DCIS status of each sample was confirmed both by examination of paraffin H & E sections of samples ((a) and (c), HE), as well as frozen cryosections ((b) and (d), FS) of the actual sample that was processed for expression profiling. (e) ‘Distinct Origins’ and ‘Evolutionary’ Theories of Breast Cancer Development. The ‘Distinct Origins’ hypothesis proposes that different molecular subtypes of cancer arise via different tumorigenic pathways, and thus constitute distinct biological entities. The ‘Evolutionary’ hypothesis proposes that the different molecular subtypes arise as a result of a single (or a few) cancer classes undergoing different stages of phenotypic development. One cannot distinguish between the two hypotheses by only studying advanced invasive cancers obtained at a single point in time.

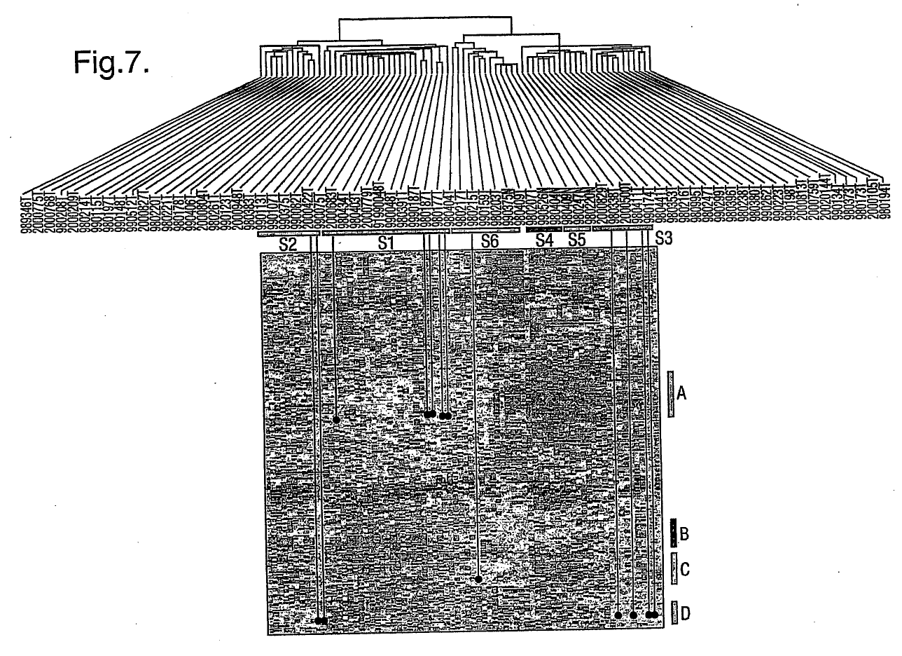

FIG. 7: DCIS samples express the hallmark genes of advanced carcinoma subtypes. DCIS samples are shown as dark vertical lines. Based upon the CIS geneset, six out of twelve DCIS samples cluster within the ERBB2+groups (S2 and S3), 5 samples in the Luminal group, and one sample was in the normal-like group. Shaded bars to the right of the clustergram represent the same gene clusters as shown in FIG. 5. (A) Luminal epithelial genes with ER. (B) Basal epithelial genes. (C) Normal breast-like genes. (D) ERBB2.

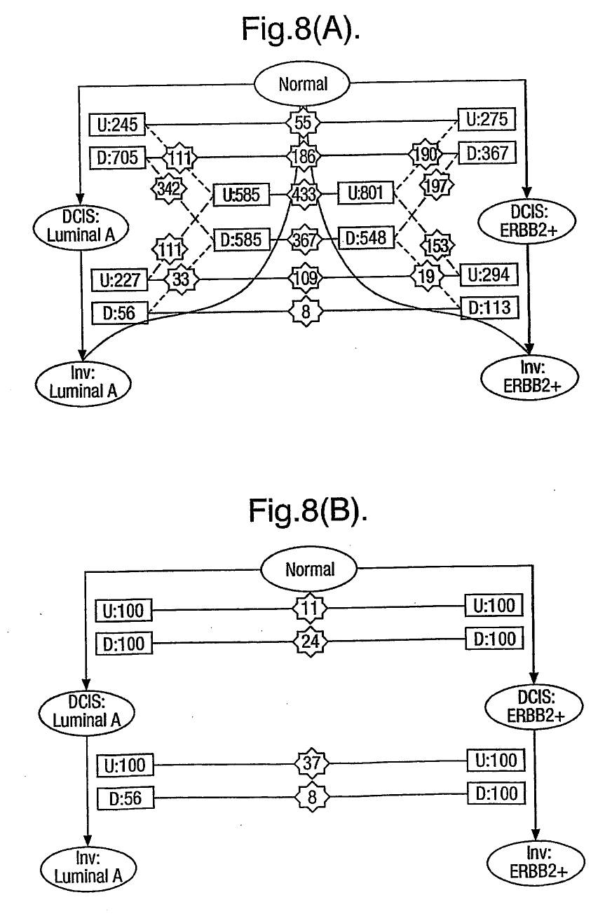

FIG. 8: Summary of pathway-specific and overlapping genes for the Luminal A and ERBB2+tumor subtypes. ‘U’ indicates upregulated genes and ‘D’ indicates downregulated genes.

For example, there are 245 genes upregulated and 705 genes downregulated during the normal/DCIS (Luminal) transition. Numbers in bold are overlapping genes between two gene sets. a) Results based upon a false-discovery rate (FDR) of 5%. b) Results when only the top 100 most significantly regulated unique genes are compared.

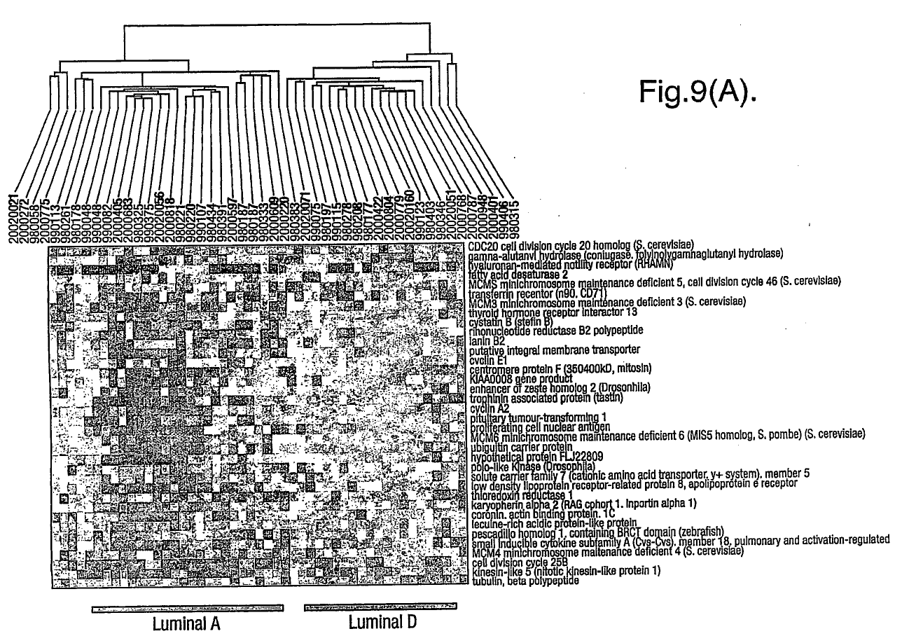

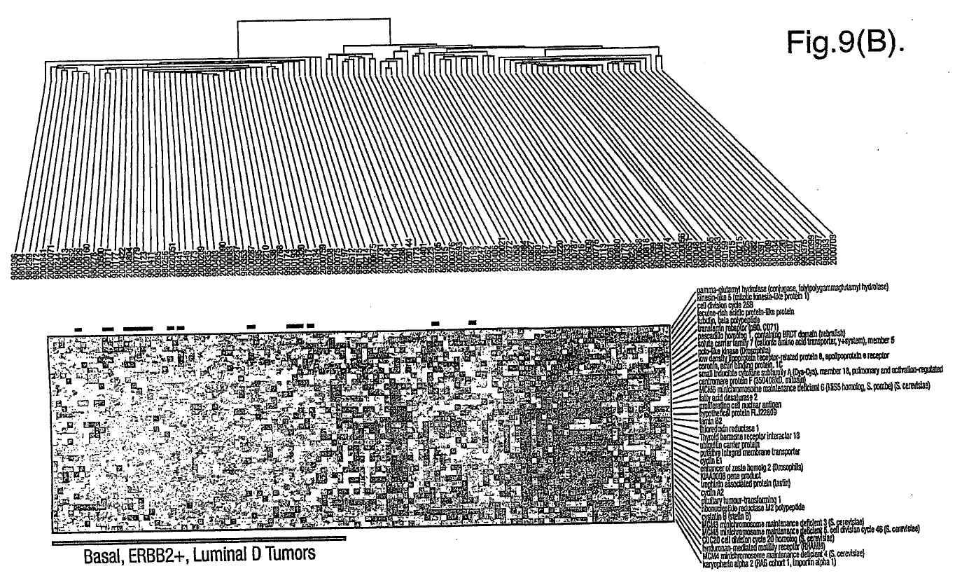

FIG. 9. a) Discovery of a Luminal D subtype. A series of previously homogenous Luminal A tumors (identified as subtype S1 by the CIS in FIGS. 5 and 7 were regrouped by hierarchical clustering based upon ‘proliferation cluster’ linked genes. Two broad groups are observed, which exhibit low (Luminal A) and high (Luminal D) levels of expression of the ‘proliferation cluster’ respectively. b) High levels of the 36-gene ‘proliferation cluster’ is also observed in other aggressive tumor types. Luminal D (15 out of 17 samples, indicated as dark bars under sample numbers), Basal (ER−) and ERBB2+ve samples all strongly express the 36-gene ‘proliferation cluster’ (bar below clustergram, left branch), while Luminal A (all but one boundary case), normal-like and normals are show low levels of expression. Light grey/white indicates upregulation, while dark grey/black indicates downregulation.

### MATERIALS AND METHODS

Breast Tissue Samples

Primary breast tissues were obtained from the NCC Tissue Repository, after appropriate approvals had been obtained from the institution's Repository and Ethics Committees. In general, all tumour and matched normal tissues were simultaneously harvested during surgical excision of the tumour. After surgical excision, the samples were immediately grossly dissected in the operating theatre, and flash-frozen in liquid N2. Histological confirmation of tumour status was subsequently provided by the Dept of Pathology at Singapore General Hospital. Samples were stored in liquid N2 until processing was performed. With the exception of 1 tumour and matched normal sample pair that came from an Indian patient, all other samples were derived from Chinese patients. Confirmation of the DCIS status of tissue samples used in this report was achieved both by conventional H & E staining of archival samples, as well as direct cryosections of the actual sample that was processed for expression profiling.

Sample Preparation and Microarray Hybridization

For hybridisations involving Affymetrix Genechips, RNA was extracted from tissues using Trizol reagent, purified through a Qiagen Spin Column, and processed for Affymetrix Genechip hybridization according to the manufacturer's instructions. For each spotted cDNA microarray hybridization 2-3 μg of total RNA was used following single-round linear amplification (Wang et al., 2000). All breast samples for the spotted cDNA microarray hybridisations were compared against a standard commercially available mRNA reference pool (Strategene) that had been similarly amplified. cDNA microarrays were fabricated following standard procedures (DeRisi et al., 1997), using cDNA clones obtained from various commercial vendors (Incyte, Research Genetics). Except where mentioned, samples were fluorescently labelled using Cy3 dye, while the reference was labelled with Cy5. Hybridizations were performed using Affymetrix U133A Genechips. After hybridization, microarray images were captured using a CCD-based microarray scanner (Applied Precision, Inc).

Data Processing and Analysis

For spotted cDNA microarray data, fluoresence intensities corresponding to individual microarrays were uploaded into a centralized Oracle 8i database. Establishment of various data sets and gene retrievals were performed using standard SQL queries. Hierarchical clustering was performed using the program Xcluster (Stanford) and visualized using the program Treeview (Eisen et al., 1998). To identify outlier genes in tumour and normal datasets, array elements were chosen which consistently exhibited greater than 3-fold regulation across 90% of all arrays for the normal dataset and 80% of all arrays for the tumour dataset. Correlation analysis was performed using the similarity metric concept employed in Golub et. al. (1999). Briefly, the similarity metrics corresponding to the normal/tumour class distinction were calculated for each gene, and the genes then sorted based on descending order of their similarity values. After being sorted by their positive and negative correlation to the class distinction, the top 10 genes from each class were chosen for subsequent cluster analysis. Principal Component Analysis (PCA) was performed by linearly transforming the gene expression matrix, which consists of a number of correlated variables, into a ‘smaller’ number of uncorrelated variables (principal components). For datasets in linear subspace, the data can be ‘compressed’ in this manner without losing too much information while simplifying the data representation. The first principal component accounts for maximum variability in the data, and each succeeding component accounts for parts of the remaining variability.

For Affymetrix Genechips, Raw Genechip scans were quality controlled using a commercially available software program (Genedata Refiner) and deposited into a central data storage facility. The expression data was filtered by removing genes whose expression was absent in all samples (ie ‘A’ calls), subjected to a log2 transformation, and normalized by median centering all remaining genes and samples. Data analysis was then performed either using the Genedata Expressionist software analysis package or using conventional spreadsheet applications. The unsupervised dataset of 1796 genes used in FIG. 1 was established by selecting genes exhbiting a standard deviation (SD) of >1 across all well-measured samples. Average-linkage hierarchical clustering, was applied by using the CLUSTER program and the results were displayed by using TREEVIEW (9). Significance analysis of microarrays (SAM) was performed essentially as described in Tusher et al., (2001) (10), using a fold-change cutoff of 2 and an appropriate delta value to cap the gene false-discovery rate (FDR) at 5% (0.05).

Creation of a Common Intrinsic Geneset (CIS)

Genes common to both the U133A Genechip Probe Set and the ‘intrinsic’ dataset as defined in Perou et al., (2000) were selected in the following manner: Out of the original ‘intrinsic’ set consisting of 456 cDNA clones, 428 could be assigned to a specific Unigene cluster using the Stanford Source database (Unigene Build 156). This number was then reduced to 403 genes after the removal of duplicate genes. The U133A Genechip probe set was then queried using this list, yielding 292 matches, or 72.5% of the original ‘intrinsic’ set (counting only unique genes).

Results

Partitioning of Normal and Tumour Breast Specimens Using Unsupervised Clustering

The inventors used cDNA microarrays of approximately 13,000 elements to generate gene expression profiles for a set of 26 grossly-dissected breast tissue specimens (14 tumour, 12 normal) obtained from patients of primarily Chinese ethnicity (see Materials and Methods). After hybridization and scanning, approximately 8,000 array elements were found to exhibit flourescence signals significantly above background levels, and these elements were used for subsequent analysis. Initially, the inventors found that an unsupervised clustering methodology based upon a number of commonly used data filters (e.g. selecting genes exhibiting at least 3-fold regulation across at least 4-5 arrays) (see Perou et al., 1999, Wang et al., 2000) resulted in an array clustergram shown in FIG. 1. Specifically, the sample set segregated into two broad groups, with each group consisting of a mixture of tumour and normal specimens. However, within each group, the inventors found that the tumour and normal tissues effectively segregated into fairly independent sub-branches. The observation that tumour and normal tissues can be segregated using unsupervised clustering suggests that specific genes may exist that can effectively distinguish between a tumour and normal sample. However, in the context of a large unsupervised data set, it is also clear that these genes are only capable of distinguishing between normal and tumour samples in sub-branches of the correlation dendogram, rather than at the level of a primary class division. Similar findings have also been reported in other breast cancer expression profiling projects (Perou et al., 2000), suggesting that at the level of global transcriptosome, the expression levels of other genes may ‘supercede’ the information encoded by genes involved in the tumour/normal class distinction (see discussion).

Use of Outlier Genesets to Classify Normal and Tumour Samples

One of the main objectives of the inventors' research is to identify genes or gene subsets that are of significant diagnostic or therapeutic potential. To be of clinical utility, it will be necessary to identify a class of genes that can accurately predict if an unknown breast tissue sample is normal or malignant at the level of the primary, rather than secondary, class division. To identify these genesets, or ‘genetic identifiers’, a number of supervised learning strategies, such as neigborhood analysis and artificial neural networks, have been previously described (Golub et al., 1999, Khan et al., 2001). However, the inventors used a slightly different strategy to identify these elements that focuses on the use of highly reproducible outlier genes. In this methodology, samples belonging to different classes are initially established as independent datasets. Within each group, genes that are consistently up or downregulated (‘outliers’) across all or close to all arrays are then identified. These separate ‘outlier groups’ are then combined, and the ability of the combined set of genes to distinguish between the two classes is then assessed using standard clustering methodologies.

The inventors first established outlier gene subsets for both the normal and tumour populations. To avoid biases that might be introduced by fluorophore labelling, they also included in each group 5 ‘reciprocal’ expression profiles in which the sample and reference RNA population were inversely labelled. This analysis identified 60 highly reproducible ‘outlier’ genes for the normal group and 75 genes for the tumour group that were either consistently up or down-regulated across all or close to all arrays (FIG. 2). A cross-comparison of the normal and tumour outlier sets revealed a number of genes in common between both sets. (Table 1), leading to a final combined outlier geneset (referred to as the COG) of 108 genes.

The COG was then used to cluster the 26 breast tissue samples. In contrast to the large-scale clustergram observed in FIG. 1, the inventors found that clustering using the genes found in the COG effectively segregated the majority of tumour and normal samples into two principal branches, with 2 mis-classifications (FIG. 2a). Specifically, 1 normal sample and 1 tumour sample were mis-assigned, and in the former case a quality check of the gene expression values revealed that this sample was associated with a number of so-called ‘missing’ values (grey bars in clustergram), which may have led to this sample being mis-classified. Nevertheless, the majority of samples were correctly grouped, suggesting that for certain datasets, ‘outlier analysis’ may serve as a simple and effective method to identify discriminating genes between distinct classes.

Definition of a Minimal Genetic Identifier for the Normal vs Tumour Class Distinction in Breast Tissues

Despite representing a dramatic reduction in the number of genes from the initial data set (8,000 to 108), the number of elements contained in the COG is still too large to be feasibly included in its entirety as part of a potential diagnostic assay. Ideally, a diagnostic geneset should consist of i) a minimal number of elements, ii) be of high predictive accuracy, and iii) represent a mixture of genes that are positively and negatively correlated to the class distinction in question. To further reduce the combined outlier geneset to its most informative elements, the inventors used correlation analysis to identify and rank genes in the COG that are most highly correlated to the tumour/normal class distinction (see Materials and Methods). The 10 most highly positively and negatively correlated genes were then assessed in their ability to accurately classify the breast samples. The inventors found that this minimal set of 20 genes, referred to as a ‘genetic identifier, accurately classified all of the normal and tumour samples (FIG. 2b and Table 2). The genes that make up the ‘genetic predictor’ represent a mixture of genes known to be involved in breast and tumour biology, as well as other genes whose role in tumour formation have not as yet been described (see discussion).

Predictive Capacity of the 20-gene ‘Genetic Identifier’

All analyses done up to this point were performed on the same ‘training’ set of 26 breast samples, and thus the predictive power of the 20-element geneset has not been addressed. To assess the robustness of this ‘genetic identifier’, the inventors followed the strategy of Golub et al (1999) and tested the ability of the minimal predictor to classify a naïve ‘test set’ of another 22 breast samples, of which 12 samples were tumours and the remaining 10 were non-malignant. In a similar fashion to the training set, they found that the 20-gene genetic identifier was also able to classify the naïve set with complete accuracy (FIG. 3b). Thus, it appears that the ability of the ‘genetic identifier to predict if a given breast sample is normal or malignant is not confined to the training-set from which it was generated. Instead, the number of elements in this geneset, although minimal, may be of sufficient sensitivity and informative power to give it predictive value.

Assessing the Global Level of Variation between Normal and Tumour Breast Tissues

Breast tumours are clinically characterized by wide variations in clinical courses, disease aggressiveness, and response to medication. Consistent with these wide phenotypic variations has been the finding that individual breast tumours can exhibit large variations in their global gene expression patterns (Perou et al., 2000). One common hypothesis to explain these wide variations is to consider them as the consequences of multiple independent pathways of tumourigenesis. However, normal breast tissues are also highly environmentally and hormonally sensitive, and the specific state of a normal breast tissue in a particular patient is often dependent upon numerous demographic factors, such as age, menopausal status, and medication history. Thus, it is formally possible that a certain amount of the variations in expression state observed in tumours may also be reflected in non-malignant breast tissue as well. Since the inventors' data set consists of both normal and malignant samples, they were able to compare the inherent variability of normal and tumour samples to each other. To perform this comparison, they utilized principal component analysis (PCA) on the entire 8,000 gene expression matrix, comprising a total of 22 non-malignant and 26 tumour specimens. Using PCA, the inventors reduced the total gene set to a series of distinct ‘components’, in which each component represents a finite amount of gene expression variation across the primary data set. They hypothesized that observed variation in the data could arise from multiple sources, such as intrinsic biological variation, as well as experimentally introduced variation (such as differences in sample harvesting, hybridization and labelling conditions, etc). However, since the normal and tumour samples were identically harvested, treated and processed in their experiments, variations due to experimental conditions and handling should be equally shared between both groups. Thus, any differences in variation between the tumour and normal groups can most likely be attributed to intrinsic biological variation.

The inventors plotted the amount of variation observed in the normal and tumour data sets against their principal components (FIG. 4). In order to effectively compare the two datasets, each component was normalized to the first component in that dataset, resulting in a graph that depicts how the total variation across the dataset “decays” with each successive principal component (By convention, the first principal component is usually taken to represent the elements that exhibit maximal variation across the dataset). The inventors observed that as a general rule, every component corresponding to the tumour data set consistently exhibited higher variation than an analogous component in the normal data set. This data indicates that the gene expression profiles of normal breast samples are significantly more ‘static’ or ‘unchanging’ when compared to tumour profiles, supporting the hypothesis that the wide variations in gene expression observed in tumours may be a consequence of breast tumours arising from multiple tumourgenic pathways.

Conservation of Molecular Subtypes of Breast Cancer Across Distinct Ethnic Populations

The inventors then used Affymetrix Genechips to profile 56 invasive breast cancers and 6 normal breast tissues that had been isolated from Chinese patients. The raw expression profile scans were subjected to one round of quality control, data filtering and processing (see Materials and Methods), and an unsupervised hierarchical clustering algorithm was used to order the normalized profiles to one another on the basis of their transcriptional similarity. Using a dataset of 1796 genes, which constitute genes that are both well-measured across at least 70% of all samples and which exhibited considerable transcriptional variation across the samples (as reflected by having a high standard deviation), the inventors observed that the majority of the samples segregated into several discernible groups that could be correlated to specific histopathological parameters. For example, many of the ER+ tumors clustered together ((S1) bar, FIG. 5a), as did the ERBB2+/ER − samples ((S3) bar). The normal breast samples also clustered as a discernible group whose individual members exhibited very high correlation to one another, suggesting that there is less transcriptional variation in normal breast tissues as compared to tumors. A number of samples, however, were not accurately segregated by the unsupervised clustering algorithm (gray bar)—it is possible that such ‘mixed clustering’ results may be attributable to ‘noise’ contributed by non-malignant components in the primary tissue sample, such as normal breast epithelial tissue, lymphocytic infiltrates, and reactive desmoplastic tissue. As previously mentioned, a similar observation was obtained using the cDNA microarray platform, suggesting that this phenomena is technology-platform independent.

One objective of this study was to determine if the molecular subtypes and associated expression signatures defined in previous published studies were also detectable in a separate patient population. The inventors focused on correlating their expression results to that of Perou et al (2000), a landmark study in which a similar analysis had been performed on a series of breast cancer specimens derived from US and Norwegian patients. Briefly, in that study and a subsequent companion report (Sorlie et al., 2001), the authors determined that invasive breast cancers could be subdivided into at least 5 distinct molecular subtypes based upon an ‘intrinsic’ geneset representing genes whose transcriptional variation is primarily due to the malignant tumor component. The specific expression signatures that represent the ‘hallmark’ elements of each particular subtype are summarized in Table 1 (this dataset is henceafter referred to as the Stanford study). Between the Stanford study and the inventors work, there are several differences in methodology and experimental design, such as differences in sample handling protocols, patient population, and expression array platform (2-color cDNA microarray in the Stanford study vs 1-color Genechips in the inventors' study, as well as different array probe sequences). The availability of two distinct breast cancer expression datasets from independent institutions (Stanford and the inventors) thus allowed the inventors to test whether, despite these differences, if the molecular subtypes defined in one institution's experiments are indeed sufficiently robust to be detectable in another institution's study.

To perform this analysis, the inventors first identified probes on the Affymetrix U133A Genechip corresponding to genes belonging to the ‘intrinsic’ set as defined by the Stanford study (see Materials and Methods). Of 403 unique genes found in the Stanford ‘intrinsic’ set, 292 genes, or 72.5% of the intrinsic set, were also found on the Genechip array. The inventors henceforth refer to this overlapping set of genes as the ‘common intrinsic set’ (CIS). Importantly, the CIS still contains many of the ‘hallmark’ genes whose transcription was reported in the Stanford study to be useful for discriminating between subtype, and reclustering of the Stanford tumors using the CIS also yielded highly similar groupings to that obtained using the full intrinsic set (data not shown). When the invasive cancers in the inventors' series were reclustered on the basis of the CIS, they observed a striking improvement in the segregation pattern where now all the cancer samples grouped into highly distinct classes. The inventors then proceeded to compare the molecular subtypes defined in their study to those discovered by the Stanford study (Luminal A, Luminal B/C, Basal, Normal-like, and ERBB2+) (Perou et al., 2000; Sorlie et al., 2001).

Luminal subtypes: All of the cancers in this group were ER + by conventional immunohistochemisty. The Stanford study defined at least two groups of luminal tumors—Luminal A and Luminal B/C, the latter being associated with a poorer clinical prognosis (Luminal B and C tumors are treated as a single class, as it is reportedly difficult to divide them into two discrete groups (Sorlie et al., 2001). Consistent with the Stanford study, the inventors also observed the presence of a robust Luminal molecular subtype that was highly similar to the Luminal A subtype of the Standford study, as this subtype was characterized by high levels of expression of ER and related genes such as GATA3, HNF3a, and X-box Binding Protein 1 (bar (S1). They could not, however, clearly determine if the Luminal B/C subtypes as defined by the Standford study were also present in their patient population, based upon the criteria that both the B/C subtypes are associated with intermediate levels of ER related gene expression, and that the luminal C subtype also expresses high levels of a ‘novel’ gene cluster. The inventors also observed the presence of a second luminal subclass (ER+/ERBB2+) which was distinct from the luminal A cancers in that this other subclass expressed intermediate levels of ER-related genes (similar to Luminal B/C) and genes found in the ‘novel’ cluster (similar to luminal C, bar (S2). This subclass, however, also expressed high levels of ERBB2-related genes, and is thus likely to be distinct from the luminal C cancers defined by the Stanford study, as luminal C cancers express low levels of the ERBB2 gene cluster. Taken collectively, the inventors' results indicate that Luminal A tumors (“Luminal in FIG. 5) constitute a robust molecular subtype that can be commonly found across different patient populations. Conversely, the luminal B/C and ER+/ERBB2 +ve subtypes may represent less robust variants whose presence may be more significantly affected by differences in ethnic specificity, sample handling protocols, or array technology.

As seen in FIG. 5, tumours belonging to the Luminal category (subtype S1) appear to be transcriptionally homogenous on the basis of the CIS. To determine if tumours belonging to this subtype could be further subdivided, the inventors reclustered a larger group of Luminal tumours using a separate set of genes which in a previous report had been shown to be indicative of a tissue's cellular proliferative status (Sorlie et al., 2001).

On the basis of these “proliferation genes”, they found that the Luminal tumours could be subdivided into two distinct types, namely, “pure” luminal A and another subtype that they have referred to as a Luminal D subtype (FIG. 9a). It is likely that the Luminal A/D subdivision is clinically meaningful, as a reclustering of a more diverse set of tumours on the basis of the “proliferation genes” resulted in two broad subdivisions, one representing clinically aggressive tumours (Basal, ERBB2 and Luminal D), and the other representing tumours that are more clinically tractable (Luminal, Normal/Normal-like) (FIG. 9b).

Basal-like: The basal molecular subtype was reported in the Stanford study to be characterized by high levels of two expression signatures—I) markers of the basal mammary epithelia, such as keratin 5 and 17, and II) genes belonging to the ‘novel’ cluster. Consistent with the Stanford study, the inventors also observed a basal subtype associated with similar expression signatures (bar(S4)), indicating that the basal molecular subtype is also highly robust. In addition, however, they also detected the apparent presence of another subtype (bar (S5)) that was not associated with any of the expression signatures described in the Stanford study.

Normal Breast-like: The ‘normal-like’ subtype is ssociated with expression of a gene cluster that is also highly expressed in normal breast tissues, and includes genes such as four and a half LIM domains 1, aquaporin 1, and alcohol dehydrogenase 2 (class I) beta. A number of tumors in the inventors' series also clustered with the normal breast tissues and exhibited this expression signature (bar (S6)). Thus, the ‘normal-like’ molecular subtype can also be considered to be a robust subtype.

ERBB2+: The Stanford study also defined a final ERBB2+ subtype in which these tumors were characterized by high levels of expression of ERBB2 related genes (column E), intermediate levels of expression of the ‘novel’ cluster (column B), and absent expression of ER-related genes (column A). A similar ERBB2+ subtype was also clearly present in the inventors' series (bar (S3)). Consistent with the expression data, they also subsequently confirmed that the tumors belonging to this molecular subtype were all ERBB2+ by conventional immunohistochemistry as well.

To summarize, of the 5 molecular subtypes defined by the Stanford study, the inventors clearly detected at least 4 subtypes in their own patient population (luminal A, basal-like, normal breast-like, and ERBB2+). They could not clearly determine if one particular subtype (luminal B/C) was present in their series using the genes in the CIS, and they also detected the potential presence of 2 additional subtypes (ER+ ERBB2+ and ER− Subtype II) which have not been reported before. The finding that that the majority (4/5) of the Stanford molecular subtypes were also clearly detectable in the inventors' study suggests that despite many methodological differences between centres, that molecular subtypes as defined by expression based genomics are indeed remarkably robust and conserved between different patient populations.

Ductal Carcinoma In Situ (DCIS) Cancers Express The Hallmark Expression Signatures of Invasive Cancer Molecular Subtypes

The previous results indicate that molecularly similar subtypes of breast cancer can indeed occur and be detected across distinct ethnic populations. One limitation of these studies, however, is that it is often very difficult to profile the same cancer over an extended period of time. As such, one question that is often raised is whether these molecular variants represent subtypes that are truly distinct biological entities, or whether they simply reflect a single or a few subtypes in different stages of evolution. Since these two different theories, referred to as the ‘distinct origins’ and the ‘evolutionary’ hypotheses respectively (FIG. 6e), have different implications for clinical diagnosis and subsequent staging and monitoring, it is thus important to determine which of these proposed mechanisms is the case for breast cancer. Unfortunately, it is not possible to distinguish between these two models by only studying invasive cancers that have been sampled at a single point in time, as both hypotheses would be expected to produce results similar to that shown in FIG. 5.

In conventional histopathology, ductal carcinoma-in-situ (or DCIS) has long been recognised as the major precursor to invasive breast cancer, and likely represents the earliest morphologically detectable malignant non-invasive breast lesion. Despite their malignant status, however, DCIS cancers are also distinct from invasive cancers in a number of respects. Clinically, DCIS cancers are treated differently from invasive cancers (DCIS cases are primarily treated with surgery with or without adjuvent radiotherapy) (Harris et al., 1997), and DCIS and invasive cancers also differ substantially in their distribution of specific cancer types (Barnes et al., 1992; Tan et al., 2002). Differences such as these raise the possibility that while DCIS cases are malignant, they may also be molecularly distinct in some respects from more advanced invasive cancers. The inventors reasoned that the ‘distinct origins’ and ‘evolutionary’ hypotheses could be tested by profiling a series of DCIS cancers and comparing their profiles to their invasive counterparts. Each hypothesis carries different predictions. If the ‘distinct origins’ hypothesis is true, then the DCIS cancers, representing ‘early’ cancers, should express many, if not all, of the hallmark expression signatures associated with their more mature invasive counterparts. Alternatively, if the ‘evolutionary’ hypothesis is correct, then one might expect that the DCIS profiles to be more closely similar to one another than to their invasive counterparts. The inventors obtained 12 DCIS tissue samples whose histopathological status was confirmed by a pathologist both using conventional H & E staining as well as frozen cryosections of the actual sample that was processed (FIGS. 2a and b).

Expression profiles of the DCIS samples were then generated and compared to their invasive counterparts. Using the CIS as a starting dataset, the inventors found that the DCIS samples segregated amongst the various invasive cancer samples into distinct categories. Specifically, 5 DCIS samples segregated into the Luminal subtype, 4 into the ER−/ER-/ERBBZT ERBB2+ subtype, 2 into the ER+/ERBB2+ subtype, and 1 into the ‘normal breastlike’ subtype. Importantly, within each subtype, each of the DCIS cancers was found to robustly express the hallmark expression signatures of its particular molecular group. Interestingly, no DCIS samples were found to cluster within the basal or ER− subtype II molecular subtypes, which is consistent with previously proposed theories that these subtypes may develop without a (or possess an extremely transient) DCIS component (Barnes et al., 1992). These results suggest that distinct breast cancer molecular subtypes are present even at the DCIS stage of breast cancer tumorigenesis, supporting the hypothesis that the subtypes represent truly distinct biological entities, possibly arising via different tumorigenic pathways (the ‘distinct origins’ hypothesis).

Genes Associated with the Normal/DCIS/Invasive Cancer Transitions Implicate Disregulation of Wnt Signaling as a Common Early Event in Breast Tumorigenesis and that Luminal A and ERBB2+ Cancers Exhibit Similar Invasion Programs

Mammary tumorigenesis can be broadly divided into two main steps: First, normal breast epithelial tissue is transformed to a malignant state via the concerted deregulation of various cellular pathways (Hahn and Weinberg, 2002). Second, to progress to an invasive cancer, several additional biological subprograms also have to be further executed, including penetration of the surrounding basement membrane, invasion of the cancer into the adjacent normal stroma, and angiogenic recruitment of endothelial vessels for tumor nourishment and maintenance (Hanahan and Weinberg, 2000). Given the molecular heterogeneity of breast cancer, one important question in the field is the extent to which the genetic programs that control these two key steps are subtype specific or commonly shared among all breast cancer subtypes.

To identify genes whose expression level was significantly different between normal breast tissues, DCIS cancers, and their invasive counterparts, the inventors used significance analysis of microarrays (SAM), a robust statistical methodology that has been used in previous reports to identify significantly regulated genes (Tusher et al., 2001). They concentrated on studying the luminal and ERBB2+ cancers, as most of the DCIS samples in their study belonged to these two molecular subtypes. First, they tested and confirmed the hypothesis that DCIS cancers, despite expressing many of the hallmarks of invasive cancers, are nevertheless still transcriptionally distinct from invasive cancers. The inventors compared 5 luminal DCIS cancers to 5 luminal invasive cancers, and determined that there existed 222 genes that were significantly regulated using a 2-fold cut-off criterion and a false-discovery rate (FDR) of 5%. In contrast, a control analysis comparing only invasive luminal A cancers which had been randomly distributed into 2 groups failed to identify any significantly regulated genes under these stringent conditions. A similar result was also obtained for DCIS and invasive cancers belonging to the ERBB2+ subtype (data not shown), indicating that significant transcriptional differences exist between DCIS and invasive cancers belonging to both the Luminal A and ERBB2+ subtypes.

SAM was then used to identify genes that were significantly regulated during either the normal/DCIS and DCIS/invasive transitions for both the luminal A and ERBB2 molecular subtypes (FDR=5%). The results are summarized in FIG. 8a. In total, for the luminal A subtype, a greater number of genes were significantly down-regulated during the normal/DCIS transition than upregulated (705 genes down vs 245 genes up), while for the DCIS/Invasive transition more genes were significantly increased in expression than decreased (56 genes down vs 277 genes up). Similarly, for the ERBB2 subtype, 367 genes were significantly downregulated and 275 genes upregulated during the normal/DCIS transition, while 113 genes were down-regulated and 294 genes upregulated during the transition from DCIS to invasive cancer.

The following provides an outline as to how the genesets of Table 4, 5, 6 and 7 were determined.

A “Genetic Identifier” that can Distinguish Between a Normal vs Tumour Breast Sample

Methodology:

Data set: 95 Breast Tissue Samples (11 Normal and 84 Tumors)

Step 1: The data for each sample was normalized by median centering each expression profile around 5000 flouresence units (the Genechip technology measures expression abundance of each gene in terms of flouresence units, from 0 to 65535)

Step 2: An intensity filter was applied such that only genes with intensity values in the range of 200 to 100,000 were retained

Step 3: A ‘Valid value’ filter was applied such that genes that were at least 70% present (ie above a minimum threshold value, usually about 200) in either normals or tumors or both were retained chosen

Step 4: A statistical T-test was performed to select genes that were differentially expressed in normal vs tumors at a confidence level of p<0.00001. This resulted in the selection of 507 genes

Step 5: Of the 507 genes, a high fold change filter was applied to select genes that exhibited large differences in expression between normal and tumor samples (2.5-fold and above). This resulted in the identification of 49 genes (up in tumors) and 81 genes (up in normals) respectively. These genes are listed in Table 4a.

Step 6: The 130 (49 and 81) genes were ranked using support vector machine gene ranking in order to rank genes in the order of their importance in being able to assign an unknown breast sample to either a tumor or normal group. This was done to arrive at a small subset of genes that can accurately predict normal from tumors. Top 32 genes gave close to 1% misclassification. The results are given in Table 4b.

Step 7: The 32 geneset was tested for its predictive accuracy in the classification of normal vs tumor samples, using leave-one-out cross-validation (LVO CV) testing. No misclassifications were observed.

Support Vector Machine (SVM) Gene Ranking

This approach is used to rank the genes in a dataset according to their importance in being able to assign an unknown sample to a particular group. Typically, the samples in the dataset are divided into a (75%) training and (25%) test set. A maximum margin hyperplane separating the two classes (eg ER+ vs ER−) is calculated for the training set.

Assuming ‘m’ genes are present in the set, the equation of maximum margin hyperplane is
H═W₁*G₁+W₂*G₂+. . . +Wᵢ*Gᵢ+. . . +Wₘ*Gₘ
Where Wᵢ's are the weights and Gᵢ's refer to the variables (genes).

Using the genes corresponding to various top ‘N’ weights (weight is indicator of importance of gene in classification) the class of all samples in the test set is predicted. The prediction rules are built for varying sets of top N genes. The above procedure is repeated 100 times and the gene ranks and misclassification rates are averaged.

“Genetic Identifiers” that can Predict the Estrogen Receptor Status and the ERBB2 Receptor Status of a Breast Tumour Sample

Methodology:

Data set: 55 invasive breast tumor samples. The individual tumors were assigned to the following groups on the basis of IHC (immunohistochemistry):
a) Estrogen receptor (ER) status: 35 ER positive and 20 ER negative samples
b) c-erbB-2 (ERBB2) status: 21 ERBB2 positive and 34 ERBB2 negative samples.

Step 1: Gene selection to identify genes that are differentially expressed between a) ER+ vs ER− tumors, and b) ERBB2+ vs ERBB2− samples. Three independent gene selection techniques were used
Significance Analysis of Microarrays (SAM), a statistical technique that uses random permutations of the expression data to estimate the ‘false discovery rate’, ie the chance at which a particular gene will be falsely called as being differentially expressed (Tusher et al., 2001). The genes are then ranked by their “relative difference”, which is similar to the ranking used in Step 6, above. The top 100 significant genes were selected.
A signal to noise (S2N) strategy was used to rank genes based on their correlation with the class distinction (either ER+/ER− or ERBB2+/ERBB2−) (Golub et al., 1999). The top 100 genes were selected.
A support vector machine (SVM) ranking strategy was used to rank the genes according to their importance in assigning a breast tumor sample to the correct class (see below). The optimal gene set (with highest accuracy) was selected.

Step 2: Common Gene Set (CGS): The genes from the 3 independent analysis were pooled, and the common genes selected by all three methods were selected. Hence these genes are method-independent and sufficiently robust to be used as a ‘genetic identifier’ to predict either the ER or ERBB2 status of a breast tumor sample.

Result:
For ER classification, the CGS contains 25 unique genes (18 up, 7 down regulated)
For ERBB2 classification, the CGS contains 26 unique genes (19 up, 7 down regulated)

The genes belonging to each CGS are listed in Table 5.

Finally, the accuracy of each CGS for tumor classification was assessed using LVO CV testing. The classification algorithm used was a Support Vector Machine (SVM). Average cross validation error rate=7.286% for ER classification (overall accuracy 92%), and 6.26% for ERBB2 classification (overall accuracy 93%).

“Genetic Identifiers” that can Predict the Molecular Subtype of a Breast Tumour Sample

Methodology

Data set: Expression Profiles for tumors belonging to the various subtypes were generated using Affymetrix U133A Genechips. The hallmark expression signatures that characterize each subtype are described above.
a) Luminal (19)
b) ERBB2 (19)
c) Basal (7)
d) ER negative type 2 (5)
e) Normal and Normal like (12)
   A. Identification of a Minimal Geneset for Classification Using a One-vs-All Support Vector Machine Approach

Step 1: The data for each sample was normalized by median centering each expression profile around 1000 flouresence units (the Genechip technology measures expression abundance of each gene in terms of flouresence units, from 0 to 65535)

Step 2: A ‘Valid value’ filter was applied such that genes that were at least 70% present (ie above a minimum threshold value, usually about 200) across all samples were chosen.

Step 3: Five different data sets were created are by leaving one of the above-mentioned groups out and combining our remaining groups (ie ‘One-vs-all’).

| Dataset | Description |
|---|---|
| 1 | Luminal (19) vs Rest (43) |
| 2 | ERBB2 (19) vs Rest (43) |
| 3 | Basal (7) vs Rest (55) |
| 4 | ER negative type 2 (5) vs Rest (57) |
| 5 | Normal and Normal like (12) vs Rest (50) |

Step 4: For each of the 5 datasets, genes were selected that exhibited a minimum 2 fold change between groups (Ratio of means was used to calculate the fold change between two groups).

The results are as follows

| | | Differentially |
|---|---|---|
| | | regulated |
| Dataset | Description | (2 fold) |
| 1 | Luminal (19) vs Rest (43) | 116 |
| 2 | ERBB2 (19) vs Rest (43) | 46 |
| 3 | Basal (7) vs Rest (55) | 318 |
| 4 | ER negative type 2 (5) vs | 309 |
| | Rest (57) | |
| 5 | Normal and Normal like (12) | 188 |
| | vs Rest (50) | |

Step 5: A support vector machine gene ranking analysis was performed for each of the five datasets to rank genes in the order of their importance in assigning an unknown breast sample to its appropriate class (e.g. ER or ERBB2 status, see above).

For datasets 1, 3, 4, and 5, a geneset was selected that yielded a 3% misclassification rate. In case the case of dataset 2 (ERBB2 vs rest), the use of all 46 genes gave a minimum of 9.7 error rate. Hence, all 46 were used in the predictor set. The predictor sets are shown in Table 6.

| | | Differentially | | |
|---|---|---|---|---|
| | | regulated | Top ‘N’ | Error |
| Dataset | Description | (2 fold) | genes | rate |
| 1 | Luminal (19) vs Rest (43) | 116 | 35 | 3 |
| 2 | ERBB2 (19) vs Rest (43) | 46 | 46 | 9.7 |
| 3 | Basal (7) vs Rest (55) | 318 | 20 | 3 |
| 4 | ER negative type 2 (5) vs | 294 | 111 | 3 |
| | Rest (57) | | | |
| 5 | Normal and Normal like | 188 | 50 | 3 |
| | (12) vs Rest (50) | | | |

Step 6: The samples were all combined into one dataset and one vs all cross-validation analysis was carried out using the various predictor sets. 100 independent iterations of 75:25 (training: test) random splits were used, resulting in an overall cross validation error rate of 5.25% (Overall accuracy 94%).

B. Identification of a Minimal Geneset for Classification Using a Genetic Algorithm/Maximum Likelihood Discriminant (GA/MLHD) Approach

The GA/MLHD approach is a different classification algorithm (Ooi & Tan, 2003) that serves as an alternative to the OVA SVM described in A.

Step 1: Samples were broken down into the following classes:

| | No. of |
|---|---|
| Class | samples |
| ER- subtype II | 5 |
| ERBB2+ | 19 |
| Normal and | 12 |
| Normal-like | |
| Luminal | 19 |
| Basal | 7 |

A truncated dataset of 1000 genes was then established by selecting genes that exhibited the largest standard deviation (SD) across all the samples.

Step 2: 24 runs of the GA/MLHD algorithm were performed on the 62 breast cancer samples based on the class distinction described in Table 4. The accuracy of the predictor sets selected by the GA/MLHD algorithm were assessed by cross-validation and independent test studies.

Details of GA/MLHD Properties:

(a) Crossover rates: 0.7, 0.8, 0.9, 1.0.
(b) Mutation rates: 0.0005, 0.001, 0.002, 0.0025, 0.005, 0.01
(c) Uniform crossover
(d) Selection: stochastic uniform sampling
(e) Predictor set size range: Rₘᵢₙ=1 and Rₘₐₓ=80.

30 optimal predictor sets with sizes ranging from 13 to 17 genes per predictor set were obtained. Each predictor set was associated with a classification accuracy of 1 error out of 62 samples. (error rate: 1.61%, overall classification accuracy 98%). 10 out of the 30 predictor sets wrongly classified the Luminal-A sample 980221T as a Normal sample. For the other 20 predictor sets, 19 misclassified the ERBB2+ sample 990262T as a ER− subtype II sample, while 1 predictor set wrongly classified the same 990262T sample as a Basal-type sample. Two of the optimal predictor sets are displayed in Table 6b.

Identification of a Luminal D Subclass in the Asian Breast Cancer Population

Previous breast cancer expression profiling studies done on primarily Caucasian populations revealed the existence of a ‘luminal’ subtype characterized by the high expression of estrogen-receptor related genes such as ESR1, GATA3, and LIV-1. Further, these ‘luminal’ cancers could be further subdivided into at least 2 further subtypes: Luminal A and Luminal B/C. While Luminal A tumors express very high levels of ER related genes, Luminal B/C cancers express intermediate levels of the ER gene cluster. Furthermore, luminal C tumors also express high levels of a ‘novel’ gene cluster. Luminal B/C tumors were found to exhibit a worse clinical prognosis than Luminal A tumors, arguing that these subtypes are indeed clinically relevant.

A similar study on breast cancers derived from Chinese patients performed in Singapore confirmed that the luminal A subtype is also present in the Asian patient population. However, the luminal B/C subtype was not detected. The reasons behind this difference may be due to methodological differences between the two studies or true differences in patient population.

A careful inspection of the original Caucasian study by the inventors subsequently revealed that Luminal C tumors are also associated with high levels of a gene cluster whose members are involved in cellular proliferation. In contrast, this ‘proliferation cluster’ is lowly expressed in Luminal A tumors. The high expression of genes in the ‘proliferation cluster’ may functionally contribute to the worse clinical prognosis associated with Luminal C tumors, as this high expression levels of this cluster is also seen in tumors belonging to the clinically aggressive ERBB2+ and basal (ER−) subtypes as well. Thus, although a luminal B/C subtype was not observed in the Asian breast cancer population, the inventors hypothesized that the genes in this ‘proliferation’ cluster could also be used to subdivide the previously homogenous Luminal A tumors found in the Asian population into distinct luminal subtypes.

Results

Identification of ‘Proliferation Cluster’ Linked-Genes on the Affymetrix U133A Genechip

In the inventor's study, the expression profiles of several breast tumors were obtained using commercially available Affymetrix U133A Genechips. Genes corresponding to the original ‘proliferation’ cluster members were then selected from the Genechip. Of the 65 genes comprising the original ‘proliferation cluster’, the inventors determined at 36 (55%) were also present on the Genechip array.

Discovery of a ‘Luminal D’ Subtype in the Asian Luminal Tumor Population

The inventors then used this 36-geneset to recluster a group of tumors which in their previous analysis had been homogenously assigned to the Luminal A subtype. As seen in FIG. 1, the 36-geneset strikingly divided the tumors into two broad groups chracterized by low and high levels of expression of the 36-geneset respectively. The former group is from henceforth referred to as the true ‘luminal A’ subtype, while the latter group is referred to as ‘luminal D’, as its expression profile is distinct from previously identified subtypes.

High Levels of Expression of the 36-Geneset is Also Observed in Other Aggressive Tumor Subtypes

To determine if Luminal D tumors are also more clinically aggressive than Luminal A tumors, the inventors then determined if high expression levels of this cluster was also observed in aggressive tumors subtypes by reclustering a larger series of their tumors using only the 36-gene ‘proliferation cluster’. As seen in FIG. 2, Luminal D tumors intermixed with tumors of the ERBB2+ and Basal subtypes, while Luminal A tumors mixed with the normal and ‘normal-like’ tumors. This result suggests that the Luminal D tumors may share certain hallmarks of more highly aggressive tumors, and that the Luminal D subtype may be clinically relevant.

A ‘Genetic Identifier’ for the Luminal D Subtype

The inventors then proceeded to develop a ‘genetic identifier’ for the Luminal D subtype. In this strategy, the ‘genetic identifier’ should only be applied to a tumor that has previously been characterized as Luminal in nature, for example by the other ‘genetic identifiers’ shown in Tables 5 and 6.

Step 1: A series of expression profiles for 19 tumors which had been previously characterized as Luminal A were normalized by median centering each expression profile around 1000 flouresence units.

Step 2: A ‘Valid value’ filter was applied such that genes that were at least 70% present (ie above a minimum threshold value, usually about 200) across all samples were chosen.

Step 3: To divide the samples in a more robust fashion, a Principal Component Analysis (PCA) was then used to ascertain the Luminal A and D subgroups using the 36 proliferation geneset (FIG. 3).

Step 4: Using the Luminal A (12 samples) vs. Luminal D (7 samples) groupings, genes were selected from the entire expression profile that exhibited a minimum 2 fold change between the two groups (Ratio of means was used to calculate the fold change between two groups). 111 such genes were identified in this analysis.

Step 5: A SVM gene ranking analysis was then performed for the 111-gene dataset to rank genes in the order of their importance in assigning a luminal breast cancer sample into either the Luminal A or Luminal D subtypes. The top 45 genes gave lowest error rate (about 12%). 18 genes were up regulated in Luminal D and 27 were down regulated in luminal D. The genes are depicted in Table 7.

Step 6: The accuracy of the 45-gene Genetic identifier was then assesed using leave one out cross validation. No misclassifications were observed.

Discussion

One outstanding challenge of the post-genomic era is to translate the huge amounts of raw sequence data generated by various genome sequencing projects into applications that improve healthcare and the treatment of disease. One area which could be revolutionised by the availability of these new resources is in the field of molecular diagnostics, where the pathologic classification of a tissue, in complementation to conventional histopathology, is also based upon a set of informative molecular markers. Importantly, one advantage of the molecular approach is that the resolving power of classification schemes based upon molecular data can be sufficiently sensitive to detect clinically relevant disease subtypes that have currently eluded traditional light microscropy approaches (Ash et al., 2000, Bittner et al., 2000).

However, before the potential of molecular diagnostics can fully realized, a number of challenges must be met and overcome. Firstly, for many common diseases, key informative genes that are able to discriminate between the relevant disease sub-classes in question must be identified. Secondly, in order to be feasibly utilized as part of a clinical assay, these genes must be ‘pared’ down to a minimal set. (‘genetic identifiers’) that collectively still delivers high predictive accuracy. Thirdly, because the clinical behaviour of many diseases can vary extensively amongst different ethnic groups and populations, it will be necessary to define appropriate limits of use of these ‘genetic identifiers’ for specific patient populations.

To address these issues, the inventors have embarked upon a large-scale expression profiling project of breast tissues derived from Asian patients. Previous reports have primarily focused on using samples derived from patients of primarily Caucasian origin (Perou et al., 2000, Gruvberger et al., 2000, Hedenfalk et al., 2000), and it is essential to determine if findings obtained from these studies will be applicable to other ethnic populations. This is especially so given the epidemiological and clinical differences in breast cancer between these distinct ethnic groups. In Caucasian populations, the majority of breast cancers tend to occur in post-menopausal women. However, in Singapore and Japan, the absolute number of breast cancer cases per year is roughly ⅓ that of the US and the incidence of breast cancer in these populations is bi-modal—the first peak, representing the majority of breast cancers, occurs in pre-menopausal women occurs at around the age of 40 (Chia et al., 2000). This first peak is then followed by a second peak at about age 55-60. The earlier incidence of breast cancer in Asian populations is unlikely to be due to earlier detection, as breast cancer screening programs in these countries are still relatively novel compared to Western countries. To explain these observations, one possibility may be that the breast cancers observed in these groups may represent distinct heterogenous subtypes arising from specific genetic or environmental differences. For example, it is known that the levels of estrogen and progesterone in Chinese women tend to be substantially lower than in Caucasians (Lippman, 1998).

To ensure maximal diversity in the repertoire of expression profiles used in the inventors' analysis, the inventors selected samples derived from patients from a wide variety of demographic and clinical backgrounds, as well as tumours of varying grades and appearances. First, the inventors identified a ‘genetic identifier’ in breast cancer for what is perhaps the most basic distinction of clinical utility—i.e. distinguishing if a given sample is ‘normal’ or ‘malignant’. Although this distinction can be currently made by a qualified pathologist using conventional histopathology, the availability of such a molecular assay would still be of use in clinical settings where rapid diagnosis is required, or when a pathologist may not be readily available. By focusing on highly reproducible ‘outlier’ genes in both normal and tumour datasets, the inventors identified a minimal set of 20 genes that is apparently able to accurately predict if an unknown breast sample is normal or malignant in both a training set and naïve test set of comparable sample quantity. In addition, using principal component analysis, they were able to show that at the expression profiles of normal breast samples appears to be far less varied than their corresponding tumour profiles. In the field of breast cancer research, there are surprisingly relatively few reports in the literature that have directly addressed the question of distinguishing between normal and tumour tissues using the relatively unbiased manner afforded by the DNA microarray approach. In one major study, it was found that that the expression profiles of normal breast tissues were sufficiently similar for them to co-segregate with each other using an unsupervised clustering methodology (Perou et al., 2000). However, in that report, the investigators also found that the normal samples, rather than segregating as an independent branch distinct from the tumour samples, instead segregated within a broad tumour class originating from mammary epithelial cells of ‘basal’ or ‘myoepithelial’ origin. This result, most likely due to the similarity of genes that are expressed in normal tissues and tumours of this subclass, illustrates that it may not be trivial to use purely unsupervised methodologies to discriminate between normal and tumour breast tissues. However, while this appears to be an issue for breast cancer genomics, it may not apply to other tissue types. For example, it appears that unsupervised clustering is able to discriminate between normal and malignant colon samples (Alon et al., 1999). One reason for this may be that colon tumours, which primarily arise from disruption of the APC/β-catenin pathway, may be genetically more uniform than breast tumours.

The genes involved in the 20-gene ‘genetic identifier’ belong to many different categories. Genes such as apolipoprotein D are well-known terminal differentiation genes in breast biology, while MAGED2 was previously isolated as a gene that is overexpressed in primary breast tumours, but not in normal mammary tissue or breast cancer cell lines (Kurt et al., 2000). Another gene, ITA3, which produces the alpha-3 subunit of the alpha-3/beta-1 integrin, has been shown to be associated with mammary tumour metastasis (Morini et al., 2000). The CAV1 protein, which links integrin signaling to the Ras/ERK pathway, has also previously been identified as a potential tumour suppressor gene (Wary et al., 1998, Weichen et al., 2001), which may explain its expression in normal breast tissues but not tumours. In addition to genes with known roles in breast and tumour biology, other intriguing genes were identified whose role in tumourgenesis is unclear or not known. For example, thrombin, best known for its role in the coagulation cascade, has recently been shown to inhibit tumour cell growth, which may explain its expression in normal but not tumour breast samples (Huang et al., 2000).

Another example is the human homolog of the S. cerevisiae PWP2 gene, which in yeast plays an essential role in cell growth and separation (Shafaatian et al., 1996).

To gain insights into the diversity of breast cancer molecular subtypes in the Asian population, the inventors then generated and analyzed a series of expression profiles of both invasive breast cancers and DCIS cancers. The aim of this work was to attempt to validate the molecular subtyping scheme defined in the Stanford study using another breast cancer expression dataset. By comparing their expression profiles to previously published studies performed using patient samples of primarily Caucasian origin, they found that the majority of molecular subtypes and hallmark expression signatures were robustly conserved between the two series. Although a similar validation study has recently been reported for prostate cancer (Rhodes et al., 2002), this report is the first time such a comparative analysis has been performed for breast cancer. The conservation of molecular subtypes between the two populations is all the more remarkable when one considers the many methodological differences existing between the studies. For example, one finding of interest was the inventors' ability to detect similar subtypes in both series despite the differences in array technology platform. This result is significant as there is currently conflicting data in the field regarding the feasibility of integrating data from different genomic expression technologies. For example, in Rhodes et al., (2002), it was reported that prostate cancer expression data from spotted cDNA arrays yielded similar data to oligonucleotide arrays.

In contrast, another recent report comparing the expression profiles of cell lines as measured by spotted and oligonucleotide arrays reported a very poor correlation between the studies (Kuo et al., 2002). The inventors' results suggest that data from different technology platforms can indeed be compared, so long as the subtype distinctions in question are fairly robust in nature. The inventors' results also suggest that despite the epidemiological differences in breast cancer between the Asian and Caucasian population (see beginning of Discussion), that breast cancers between the ethnic groups are to a first approximation highly molecularly similar.

The inventors also found that DCIS cancers robustly express many subtype-specific gene expression signatures, suggesting that these molecular subtypes can be discerned even at this pre-invasive stage. Thus, it is unlikely that these subtypes represent an evolving cancer class, but are distinct biological entities that may posses different tumorigenic origins. Despite the expression of subtype-specific expression signatures in DCIS cancers (as reported in this study), there is other evidence in the field that DCIS cancers may be distinct from invasive cancers. For example, previous retrospective reports have shown that the majority of low nuclear grade DCIS tumors undergo a long clinical evolution to invasive cancer (Page et al., 1982; Betsill et al., 1978; and Rosen et al., 1980), suggesting that additional genetic events must occur before they become invasive. In addition, histopathological studies have found that there is a considerable difference in the histopathological distribution of tumor types in DCIS cancers vs invasive cancers, with ERBB2+cancers being much more highly represented in DCIS compared to invasive cases (Barnes et al., 1992). It has been unclear, however, if this observation should be interpreted to mean that that the ER-ERBB2− cancers lack a DCIS component, or if the ERBB2+ cancers will eventually evolve to a ERBB2− state. The distinctive segregation of the DCIS cancers in the inventors' series suggests that the former is true, since the ERBB2+ cancers already express many ERBB2+ invasive hallmarks.

Finally, by integrating the expression profiles of normal, DCIS, and invasive cancers belonging to the luminal A and ERBB2+subtypes, the inventors were able to define sets of genes which were regulated in a common and subtype-specific manner during the normal, DCIS, and invasive cancer transitions. Although the results of these analyses clearly need to be supported by further experimental work before any definitive conclusions can be made, there were a number of intriguing observations. The inventors found that a number of components of the Wnt signaling pathway were commonly regulated during the transition from normal —>DCIS for both subtypes, implicating deregulation of Wnt signaling as an important common event in breast cancer carcinogenesis. Although previous reports have reported the involvement of the Wnt pathway in human breast cancer carcinogenesis (Smalley et al., 2001), it has been less clear if this is an early or late event. The inventors' results suggest the former possibility is more likely.

Secondly, the remarkable commonality of genes regulated from the DCIS to the invasive stage between the two subtypes suggests that many of the genetic processes that underlie cellular invasion, desmoplastic reaction, stromal remodeling etc, may be fairly general and shared across different breast cancer subtypes. Finally, the inventors' results also suggest that both cancer subtypes may be highly metabolically distinctive, with ERBB2+ tumors having a greater reliance on ionic-related processes, while Luminal A tumors may be under a state of chronic metabolic stress. These results are extremely important, for example, the increased metabolic load of Luminal A tumors may explain why ER+ tumors are more radiosensitive than ER− tumors (Villalobos et al., 1996), and calcium signaling may play a role in tumor cell motility controlled by the ERBB2+ receptor (Feldner and Brandt (2002).

### REFERENCES

Alon, U., N. Barkai, D. A. Notterman, K. Gish, S. Ybarra, D. Mack, and A. J. Levine (1999) Broad patterns of gene expression revealed by clustering analysis of tumour and normal colon tissues probed by oligonucleotide arrays. Proc Natl Acad Sci 96,
Ash, A. A., M. B. Eisen, R. E. Davis, C. Ma, I. S. Lossos, A. Rosenwald, J. C. Boldrick, H. Sabet, T. Truc, Y. Xin, J. I. Powell, L. Yang, G. E. Marti, T. Moore, J. Hudson, L. Lisheng, D. B. Lewis, R. Tibshirani, G. Sherlock, W. C. Chan, T. C. Greiner, D. D. Weisenburger, J. O. Armitage, R. Warnke, R. Levy, W. Wilson, M. R. Grever, J. C. Byrd, D. Botstein, P. O. Brown, and L. M. Staudt (2000) Distinct types of diffuse large B-cell lymphoma identified by gene expression profiling. Nature 403, 503-511
Barnes, D. M., J. Bartkova, R. S. Champlejon, W. J. Gullick, P. J. Smith, and R. R. Millis (1992) Overexpression of c-erbB2 Oncoprotein: Why does this occur more frequently in ductal carcinoma in situ than in invasive mammary carcinoma and is this of prognostic significance? Eur J Cancer 28, 644-648
Betsill, W. L. J., P. P. Rosen, P. H. Lieberman, and G. F. Robbins (1978) Intraductal carcinoma. Long-term follow-up after treatment by biopsy alone. JAMA 239, 1863-1867
Bittner, M., P. Meltzer, Y. Chen, Y. Jiang, E. Seftor, M. Hendeix, M. Radmacher, R. Simon, Z. Yakhini, A. Ben-Dor, N. Sampas, E. Dougherty, E. Wang, F. Marincola, C. Gooden, J. Lueders, A. Glatfelter, P. Pollock, J. Carpten, E. Gillanders, D. Leja, K. Dietrich, C. Beaudry, M. Berens, D. Alberts, V. Sondak, N. Hayward, and J. Trent (2000) Molecular classification of cutaneous malignant melenoma by gene expression profiling. Nature 406, 536-540
Chia, K. S., A. Seow, H. P. Lee, and K. Shanmugaratnam (2000) Cancer Incidence in Singapore, 1993-1997. In (Singapore Cancer Registry)
DeRisi, J. L., V. R. Iyer, and P. O. Brown (1997) Exploring the Metabolic and Genetic Control of Gene Expression on a Genomic Scale. Science 278, 680-686
Eisen, M. B., P. T. Spellman, P. O. Brown, and D. Botstein (1998) Cluster analysis and display of genome-wide expression patterns. Proc Natl Acad Sci 95, 14863-14868
Feldner, J. C. and B. H. Brandt (2002) Cancer cell motility—on the road from c-erbB-2 receptor steered signaling to actin reorganization. Exp Cell Res 272, 93-108
Giuliano, A. E. (1998) Breast. In Current Medical Diagnosis and Treatment, 37, Ed. Tierney, L. M. S. J. McPhee and M. A. Papadakis (Appleton and Lange, Stamford) 666-690
Golob, T. R., D. K. Slonim, P. Tamayo, C. Huard, J. P. Gaasenbeek, H. Coller, M. L. Loh, J. R. Downling, M. A. Caligiuri, C. D. Bloomfield, and E. S. Lander (1999) Molecular Classification of Cancer: Class Discovery and Class Prediction by Gene Expression Monitoring. Science 286, 531-537
Gruvberger, S., M. Ringner, Y. Chen, S. Panavally, L. H. Saal, A. Borg, M. Ferno, C. Peterson, and P. Meltzer (2001) Estrogen Receptor Status in Breast Cancer is Associated with Remarkably Distinct Gene Expression Patterns. Cancer Research 61, 5979-5984
Hahn, W. C. and R. A. Weinberg (2002) Rules for making human tumor cells. N Engl J Med 347, 1593-1603
Harris, J. R., M. Morrow, and L. Norton (1997) Malignant Tumors of the Breast. In Cancer: Principles and Practice of Oncology, Ed. Devita, V. T. S. Hellman and S. A. Rosenberg (Lippincott-Raven, Philadelphia/N.Y.)
Hanahan, D. and R. A. Weinberg (2000) The hallmarks of Cancer. Cell 100, 57-70

Hedenfalk, I., D. Duggan, Y. Chen, M. Radmacher, M. Bittner, R. Simon, P. Meltzer, B. Gusterson, M. Esteller, O. P. Kallioniemi, M. Wilfond, A. Borg, and J. Trent (2001) Gene Expression Profiles in Hereditary Breast Cancer. NEJM 344, 539-548
Huang, Y., J. Li, and S. Karpatkin (2000) Thrombin inhibits tumour cell growth in association with up-regulation of p21(wafl/cipl) and Caspases via a p53-independent, STAT-1-dependent pathway. J. Biol. Chem. 275, 6462-6488
Khan, J., J. s. Wei, M. Ringner, L. H. Saal, M. Ladanyi, F. Westermann, F. Berthold, M. Schwab, C. R. Antonescu, C. Peterson, and P. S. Meltzer (2001) Classification and diagnostic prediction of cancers using gene expression profiling and artificial neural networks. Nature Med 7, 673-679
Kurt, R. A., W. J. Urba, and D. D. Schoof (2000) Isolation of genes overexpressed in freshly isolated breast cancer specimens. Breast Cancer Res. Treat. 59, 41-48
Kuo, W. P., T. K. Jenssen, A. J. Butte, L. O. Machado, and I. S. Kohane (2002) Analysis of measured mRNA measurements from two different microarray technologies. Bioinformatics 18, 405-412
Kuukasjarvi, T., J. Kononen, H. Helin, K. Holli, and J. Isola (1996) Loss of estrogen receptor in recurrent breast cancer is asociated with poor response to endocrine therapy. J. Clin. Oncol. 14, 2584-2589
Lippman (1998) Breast Cancer. In Harrison's Principles of Internal Medicine, 91, Ed. Fauci, A. S. E. Braunwald K. J. Isselbacher J. D. Wilson J. B. Martin D. L. Kasper S. L. Hauser and D. L. Longo (McGraw-Hill, New York) 562-568
Morini, M., M. Mottolese, N. Ferrari, G. Ghiorzo, S. Buglioni, R. Mortarini, D. M. Noonon, P. G. Natali, and A. Albini (2000) The alpha-3 beta 1 integrin is associated with mammary carcinoma cell metastasis, invation, and gelatinase B (MMP-9) activity. Int J Cancer 87, 336-342
Ooi C. H. and Patrick Tan (2003). Genetic algorithms applied to multi-class prediction for the analysis of gene expression data. Bioinformatics. 19, 37-44.
Page, D., W. Dupont, L. Rogers, and M. Landenberger (1982) Intraductal carcinoma of the breast: follow-up after biopsy only. Cancer 49, 751-758.
Parl, F. F. (2000) Estrogens, Estrogen Receptor, and Breast Cancer. (IOS Press)
Perou, C. M., S. S. Jeffrey, M. van de Rijn, C. A. Rees, M. B. Eisen, D. T. Ross, A. Pergemenschikov, C. F. Williams, S. X. Zhu, J. C. F. Lee, D. Lashkari, D. Shalon, P. O. Brown, and D. Botstein (1999) Distinctive gene expression patterns in human mammary epithelial cells and breast cancers. Proc Natl Acad Sci 96, 9212-9217
Perou, C. M., T. Sorlie, M. B. Eisen, v. d. R. M., S. S. Jeffrey, C. A. Rees, J. R. Pollack, D. T. Ross, H. Johnsen, L. A. Akslen, O. Fluge, A. Pergamenschikov, C. Williams, S. X. Zhu, P. E. Lonning, A. L. Borresen-Dale, P. O. Brown, and D. Botstein (2000) Molecular Portraits of Human Breast Tumours. Nature 406, 747-752
Rhodes, D. R., T. R. Barrette, M. A. Rubin, D. Ghosh, and A. M. Chinnaiyan (2002) Meta-analysis of Microarrays: Interstudy Validation of Gene Expression Profiles Reveals Pathway Dysregulation in Prostate Cancer. Cancer Research 62, 4427-4433
Rosen, P., D. Braun, and D. Kinne (1980) The clinical significance of pre-invasive breast carcinoma. Cancer 46, 919-925
Shafaatian, R., M. A. Payton, and J. D. Reid (1996) PWP2, a member of the WD-repeat family of proteins, is an essential Saccharomyces cerevisiae gene involved in cell separation. Mol Gen Genet. 252, 101-114
Smalley, M. J. and T. C. Dale (2001) Wnt signaling and mammary tumorigenesis. J Mammary Gland Biol Neoplasia 6, 37-52
Sorlie, T., C. M. Perou, R. Tibshirani, T. Aas, S. Geisler, H. Johnsen, T. Hastie, M. B. Eisen, M. van de Rijn, S. S. Jeffrey, T. Thorsen, H. Quist, J. C. Matese, P. O. Brown, D. Botstein, P. E. Lonning, and A. L. Borresen-Dale (2001) Gene Expression Patterns of Breast Carcinomas Distinguish Tumor Subclasses with Clinical Implications. Proc. Natl. Acad. Sci. 98, 10879-10874
Tan, P. H., K. L. Chuah, G. Chiang, C. Y. Wong, F. Dong, and B. H. Bay (2002) Correlation of p53 and cerbB2 expression and hormonal receptor status with clinicopathological parameters in ductal carcinoma in situ of the breast. Oncology Reports 9, 1081-1086
Tavassoli, F. A. and S. J. Schnitt (1992) Pathology of the Breast. In (Elsevier)
Tusher, V. G., R. Tibshirani, and G. Chu (2001) Significance Analysis of Microarrays Applied to the Ionizing Radiation Response. Proc. Natl. Acad. Sci. 98, 5116-5121
van't Veer, L. J., H. Dai, M. J. van de Vijver, Y. D. He, A. A. M. Hart, M. Mao, H. L. Peterse, K. van der Kooy, M. J. Marton, A. T. Witteveen, G. J. Schreiber, R. M. Kerkhoven, C. Roberts, P. S. Linsley, R. Bernards, and S. H. Friend (2002) Gene expression profiling predicts clinical outcome of breast cancer. Nature 415, 530-536
Villalobos, M., d. Becerra, M. I. Nunez, M. T. Valenzuela, E. Siles, N. Olea, V. Pedraza, and J. M. Ruiz de Almodovar (1996) Radiosensitivity of human breast cancer cell lines of different hormonal responsiveness. Modulatory effects of oestradiaol. Int J Radiat Biol 70, 161-169
Wang, E., L. D. Miller, G. A. Ohnmacht, E. T. Liu, and F. M. Marincola (2000) High-fidelity mRNA amplification for gene profiling. Nature Biotech. 18, 457-459
Wary, K. K., A. Mariotti, c. Zurzolo, and F. G. Giancotti (1998) A requirement for caveolin-1 and associated kinase Fyn in integrin signaling and anchorage-dependent cell growth. Cell 94, 625-634

Wiechen, K., L. Diatchenko, A. Agoulnik, K. M. Scharff, H. Schober, K. Arlt, B. Zhumabayeva, P. D. Siebert, M. Dietel, R. Schafer, and C. Sers (2001) Caveolin-1 is down-regulated in human ovarian carcinoma and acts as a candidate tumour suppressor gene. Am J Pathol. 159, 1635-1643

TABLE 1
Common Genes in Both Normal and Tumour Datasets

| | Unigene | Accession | | |
|---|---|---|---|---|
| NCC ID | ID | No | GeneName | Annotation |
| 2914401 | Hs.151738 | NM_004994 | MMP9 | matrix metalloproteinase 9 (gelatinase B, 92 |
| | | | | kD gelatinase, 92 kD type IV collagenase) |
| 2957001 | Hs.50758 | BF239180 | SMC4L1 | SMC4 (structural maintenance of chromosomes |
| | | | | 4, yeast)-like 1 |
| 3080701 | Hs.279009 | BF679062 | MGP | matrix Gla protein |
| 3080801 | Hs.98428 | NM_018952 | HOXB6 | homeo box B6 |
| 3082201 | Hs.211573 | NM_005529 | HSPG2 | heparan sulfate proteoglycan 2 (perlecan) |
| 3085601 | Hs.156110 | AW404507 | IGKC | immunoglobulin kappa constant |
| 3119301 | Hs.78045 | NM_001615 | ACTG2 | actin, gamma 2, smooth muscle, enteric |
| 3174801 | Hs.95972 | BE892678 | SILV | silver (mouse homolog) like |
| 3296301 | Hs.153952 | AW072424 | NT5 | 5′ nucleotidase (CD73) |
| 3390901 | Hs.572 | X02544 | ORM1 | orosomucoid 1 |
| 3401301 | Hs.155421 | AA334619 | AFP | alpha-fetoprotein |
| 3404301 | Hs.25817 | AW195430 | BTBD2 | BTB (POZ) domain containing 2 |
| 3437301 | Hs.78771 | AI525579 | PGK1 | phosphoglycerate kinase 1 |
| 3451301 | Hs.56205 | AW663903 | INSIG1 | insulin induced gene 1 |
| 3610001 | Hs.30743 | AI017284 | PRAME | preferentially expressed antigen in melanoma |
| 3617301 | Hs.10842 | AF052578 | RAN | RAN, member RAS oncogene family |
| 3619101 | Hs.337764 | AB038162 | NA | trefoil factor 1 |
| 3767201 | Hs.274184 | AF207550 | TFE3 | transcription factor binding to IGHM enhancer 3 |
| 3812201 | Hs.914 | X03100 | AGL | Human mRNA for SB classII histocompatibility |
| | | | | antigen alpha-chain |
| 3955201 | Hs.19710 | H60423 | SLC17A2 | solute carrier family 17 (sodium phosphate), |
| | | | | member 2 |
| 4021001 | Hs.2055 | AA232386 | UBE1 | ubiquitin-activating enzyme E1 |

TABLE 2
Genes found in the minimal breast cancer genetic identifier

| | | Accession | | | On in |
|---|---|---|---|---|---|
| NCC ID | Unigene ID | No | Genename | Annotation | Tumour |
| 2920901 | Hs.76530 | AU121309 | F2 | coagulation factor II (thrombin) | N |
| 2933601 | Hs.278411 | AB014509 | NCKAP1 | NCK-associated protein 1 | N |
| 2934801 | Hs.79380 | AP001753 | PWP2H | PWP2 homolog | N |
| 2936101 | Hs.1940 | AV733563 | CRYAB | crystallin, alpha B | N |
| 2987501 | Hs.75736 | J02611 | APOD | apolipoprotein D | N |
| 3041201 | Hs.295944 | BG621010 | TFPI2 | tissue factor pathway inhibitor 2 | N |
| 3110601 | Hs.74034 | BG541572 | CAV1 | caveolin 1, caveolae protein, 22 kD | N |
| 3119401 | Hs.184411 | AL558086 | ALB | albumin | N |
| 3143701 | Hs.156346 | NM_001067 | TOP2A | topoisomerase (DNA) II alpha (170 kD) | N |
| 3401301 | Hs.155421 | AA334619 | AFP | alpha-fetoprotein | N |
| 2919801 | Hs.177766 | BE740909 | ADPRT | ADP-ribosyltransferase (NAD+; poly | Y |
| | | | | (ADP-ribose) polymerase) | |
| 2930501 | Hs.265829 | D01038 | ITGA3 | integrin, alpha 3 (antigen CD49C, | Y |
| | | | | alpha 3 subunit of VLA-3 receptor) | |
| 2961201 | Hs.4437 | AU131942 | RPL28 | ribosomal protein L28 | Y |
| 3048301 | Hs.4943 | BE891065 | MAGED2 | hepatocellular carcinoma associated | Y |
| | | | | protein; breast | |
| | | | | cancer associated gene 1 | |
| 3085601 | Hs.156110 | AW404507 | IGKC | immunoglobulin kappa constant | Y |
| 3119301 | Hs.78045 | NM_001615 | ACTG2 | actin, gamma 2, smooth muscle, | Y |
| | | | | enteric | |
| 3124401 | Hs.145279 | NM_003011 | SET | SET translocation (myeloid | Y |
| | | | | leukemia-associated) | |
| 3134101 | Hs.73885 | 088244 | HLA-G | HLA-G histocompatibility antigen, | Y |
| | | | | class I, G | |
| 3193001 | Hs.84298 | BE741354 | CD74 | CD74 antigen (invariant polypeptide | Y |
| | | | | of major histocompatibility complex, | |
| | | | | class II antigen-associated) | |
| 3296401 | Hs.183601 | U70426 | RGS16 | regulator of G-protein signalling 16 | Y |

Genes are ordered according to their correlation to the tumour/normal class distinction.

TABLE 3
Tabulation of expression signatures associated with breast tumor subtypes. Subclasses
include Luminal A (L-A_, Luminal B (L-B), Luminal C (L-C_, Basal (Bas),
Normal like (Nor), ERBB2 (ERB). Levels of expression are indicated by H (high
expression), I (intermediate expression), and A (absent expression).

| | | Tumor subtype | | | | | |
|---|---|---|---|---|---|---|---|
| Expression Signature | Unigene | L-A | L-B | L-C | Bas | Nor | ERB |
| Luminal Epithelium | | H | I | I | A | A | A |
| estrogen receptor 1 | Hs.1657 | | | | | | |
| GATA binding protein 3 | Hs.169946 | | | | | | |
| LIV-1 | Hs.79136 | | | | | | |
| Xbox binding protein 1 | Hs.149923 | | | | | | |
| Hepatocyte Nuclear Factor 3 alpha | Hs.299867 | | | | | | |
| Basal Epithelium | | A | A | A | H | H | A |
| Keratin5 | Hs.195850 | | | | | | |
| Keratin17 | Hs.2785 | | | | | | |
| Laminin gamma 2 | Hs.54451 | | | | | | |
| Fatty acid binding protein 7 | Hs.26770 | | | | | | |
| erbb2 related genes | | A | A | A | A | A | H |
| C-ERB-B2 | Hs.323910 | | | | | | |
| GRB7 | Hs.86859 | | | | | | |
| TIAF1 | Hs.75822 | | | | | | |
| TRAF4 | Hs.8375 | | | | | | |
| Normal breast like | | A | A | A | A | H | A |
| CD36 antigen collagen type 1 receptor | Hs.75613 | | | | | | |
| Four and a half LIM domain 1 | Hs.239069 | | | | | | |
| vascular adhesion protein 1 | Hs.198241 | | | | | | |
| alcohol dehydrogenase 2 class 1 | Hs.4 | | | | | | |
| Novel | | A | A | H | H | A | I |
| kinesin-like 5 mitotic kinesin-like protein 1 | Hs.270845 | | | | | | |
| putative integral membrane transporter | Hs.296398 | | | | | | |
| gamma-glutamyl hydrolase conjugase | Hs.78619 | | | | | | |
| squalene epoxidase | Hs.71465 | | | | | | |

TABLE 4a
Set of 49 Genes Upregulated in Tumors and 81 Genes Upregulated in Normals
Upregulated in tumors

| | | | | Normal_{—} | Tumor_{—} | Fold change | |
|---|---|---|---|---|---|---|---|
| Probe | Gene Description | UniGene | GeneBank | median | median | (normal/tumor) | P-value |
| 221730_at | collagen, type V, alpha 2 | Hs.82985 | NM_000393.1 | 2989.34 | 22050.38 | 0.135568639 | 6.53E−08 |
| 205483_{—} | interferon-stimulated | Hs.833 | NM_005101.1 | 3440.12 | 19587.87 | 0.175625017 | 2.89E−09 |
| s_at | protein, 15 kDa | | | | | | |
| 201422_at | interferon, gamma- | Hs.14623 | NM_006332.1 | 4216.08 | 22685.34 | 0.185850421 | 5.13E−11 |
| | inducible protein 30 | | | | | | |
| 202311_{—} | collagen, type I, alpha 1 | Hs.172928 | NM_000088.1 | 2309.8 | 11583.18 | 0.199409834 | 5.47E−08 |
| s_at | | | | | | | |
| 214290_{—} | H2A histone family, | Hs.795 | AA451996 | 8270.53 | 34668.82 | 0.238558163 | 0.000011 |
| s_at | member O | | | | | | |
| 204170_{—} | CDC28 protein kinase 2 | Hs.83758 | NM_001827.1 | 2364.5 | 9307.97 | 0.254029611 | 2.44E−09 |
| s_at | | | | | | | |
| 204620_{—} | chondroitin sulfate | Hs.81800 | NM_004385.1 | 8494.23 | 31700.6 | 0.267951711 | 1.64E−10 |
| s_at | proteoglycan 2 (versican) | | | | | | |
| 201261_{—} | biglycan | Hs.821 | BC002416.1 | 3832.74 | 14200.24 | 0.269906706 | 2.96E−10 |
| x_at | | | | | | | |
| 221731_{—} | chondroitin sulfate | Hs.81800 | J02814.1 | 10044.24 | 36814.75 | 0.272831949 | 1.97E−09 |
| x_at | proteoglycan 2 (versican) | | | | | | |
| 203936_{—} | matrix metalloproteinase 9 | Hs.151738 | NM_004994.1 | 2908.93 | 10635.99 | 0.273498753 | 1.4E−06 |
| s_at | (gelatinase B, 92 kD | | | | | | |
| | gelatinase, 92 kD type IV | | | | | | |
| | collagenase) | | | | | | |
| 213909_at | Homo sapiens cDNA FLJ12280 | Hs.288467 | AU147799 | 2270.33 | 8261.75 | 0.274800133 | 2.93E−07 |
| | fis, clone MAMMA1001744 | | | | | | |
| 204619_{—} | chondroitin sulfate | Hs.81800 | BF590263 | 1679.69 | 5982.22 | 0.280780379 | 4.7E−07 |
| s_at | proteoglycan 2 (versican) | | | | | | |
| 213905_{—} | biglycan | Hs.821 | AA845258 | 5025.39 | 17320.39 | 0.290143005 | 6.45E−10 |
| x_at | | | | | | | |
| 203362_{—} | MAD2 mitotic arrest | Hs.79078 | NM_002358.2 | 1126.73 | 3794.7 | 0.296922023 | 4.29E−07 |
| s_at | deficient-like 1 (yeast) | | | | | | |
| 209596_at | adlican | Hs.72157 | AF245505.1 | 9872.98 | 31833.51 | 0.310144247 | 9.57E−06 |
| 217762_{—} | RAB31, member RAS oncogene | Hs.223025 | BE789881 | 6239.5 | 20080.05 | 0.310731298 | 8.96E−07 |
| s_at | family | | | | | | |
| 212353_at | sulfatase FP | Hs.70823 | AW043713 | 3298.13 | 10610.47 | 0.310837314 | 2.29E−07 |
| 221729_at | collagen, type V, alpha 2 | Hs.82985 | NM_000393.1 | 8089.9 | 25965.7 | 0.311561021 | 1.79E−08 |
| 202503_{—} | KIAA0101 gene product | Hs.81892 | NM_014736.1 | 4140.8 | 13277.67 | 0.311861946 | 8.17E−09 |
| s_at | | | | | | | |
| 200660_at | S100 calcium binding | Hs.256290 | NM_005620.1 | 19359.81 | 60412.84 | 0.320458532 | 1.37E−08 |
| | protein A11 (calglzzarin) | | | | | | |
| 210046_{—} | isocitrate dehydrogenase 2 | Hs.5337 | U52144.1 | 6598.83 | 20503.1 | 0.321845477 | 2.19E−06 |
| s_at | (NADP+), mitochondrial | | | | | | |
| 218039_at | nucleolar protein ANKT | Hs.279905 | NM_016359.1 | 2649.43 | 8088.17 | 0.327568535 | 4.71E−08 |
| 200838_at | cathepsin B | Hs.297939 | NM_001908.1 | 8903.1 | 26015.64 | 0.342221064 | 5.79E−09 |
| 208850_{—} | Thy-1 cell surface antigen | Hs.125359 | AL558479 | 3334.94 | 9742.28 | 0.342316172 | 1.02E−07 |
| s_at | | | | | | | |
| 215438_{—} | G1 to S phase transition 1 | Hs.2707 | BE906054 | 3749.34 | 10880.78 | 0.344583752 | 2.4E−07 |
| x_at | | | | | | | |
| 213274_{—} | cathepsin B | Hs.297939 | BE875786 | 5290.88 | 15121.92 | 0.349881497 | 9.49E−10 |
| s_at | | | | | | | |
| 214352_{—} | v-Ki-ras2 Kirsten rat | Hs.351221 | BF673699 | 8905.97 | 25327.68 | 0.351629916 | 4.28E−13 |
| s_at | sarcoma 2 viral oncogene | | | | | | |
| | homolog | | | | | | |
| 208691_at | transferrin receptor | Hs.77356 | BC001188.1 | 10599.34 | 30095.24 | 0.352193237 | 1.63E−06 |
| | (p90, CD71) | | | | | | |
| 211161_{—} | collagen, type III, | Hs.119571 | AF130082.1 | 16874.98 | 47522.98 | 0.355090948 | 4.8E−07 |
| s_at | alpha 1 (Ehlers-Danlos | | | | | | |
| | syndrome type IV, | | | | | | |
| | autosomal dominant) | | | | | | |
| 200887_{—} | signal transducer and | Hs.21486 | NM_007315.1 | 11865.1 | 33057.82 | 0.358919614 | 2.31E−07 |
| s_at | activator of transcription | | | | | | |
| | 1, 91 kD | | | | | | |
| 222077_{—} | Rac GTPase activating | Hs.23900 | AU153848 | 2198.49 | 6100.35 | 0.360387519 | 1.65E−08 |
| s_at | protein 1 | | | | | | |
| 212057_at | KIAA0182 protein | Hs.75909 | D80004.1 | 5085.42 | 14109.59 | 0.360422946 | 9.01E−06 |
| 222039_at | hypothetical protein | Hs.274448 | AA292789 | 985.61 | 2733.2 | 0.360806615 | 6.79E−06 |
| | FLJ11029 | | | | | | |
| 202391_at | brain abundant, membrane | Hs.79516 | NM_006317.1 | 6613.73 | 18202.02 | 0.36335143 | 1.85E−06 |
| | attached signal protein 1 | | | | | | |
| 222158_{—} | CGI-146 protein | Hs.42409 | AF229834.1 | 2670.29 | 7278.07 | 0.366895345 | 1.63E−06 |
| s_at | | | | | | | |
| 214435_{—} | v-ral simian leukemia | Hs.288757 | NM_005402.1 | 1882.24 | 5097.71 | 0.369232459 | 2.9E−09 |
| x_at | viral oncogene homolog | | | | | | |
| | A (ras related) | | | | | | |
| 208998_at | uncoupling protein 2 | Hs.80658 | U94592.1 | 10979.98 | 29619.79 | 0.370697429 | 2.5E−08 |
| | (mitochondrial, | | | | | | |
| | proton carrier) | | | | | | |
| 205436_{—} | H2A histone family, | Hs.147097 | NM_002105.1 | 4050.78 | 10910.21 | 0.371283413 | 2.31E−08 |
| s_at | member X | | | | | | |
| 209218_at | squalene epoxidase | Hs.71465 | AF098865.1 | 4862.95 | 12883.73 | 0.377448922 | 2.68E−06 |
| 219148_at | T-LAK cell-originated | Hs.104741 | NM_018492.1 | 783.67 | 2061.19 | 0.380202698 | 1.27E−05 |
| | protein kinase | | | | | | |
| 214710_{—} | cyclin B1 | Hs.23960 | BE407516 | 1750.12 | 4576.64 | 0.382402811 | 1.41E−06 |
| s_at | | | | | | | |
| 202736_{—} | U6 snRNA-associatad | Hs.76719 | NM_012321.1 | 3258.86 | 8432.11 | 0.38648215 | 7.8E−07 |
| s_at | Sm-like protein | | | | | | |
| 201954_at | actin related protein | Hs.11538 | NM_005720.1 | 5792.32 | 14857.02 | 0.389870916 | 1.98E−09 |
| | ⅔ complex, | | | | | | |
| | subunit 1B (41 kD) | | | | | | |
| AFFX- | | | | | | | |
| HUMISGF3A/ | | | | | | | |
| M97935_{—} | signal transducer and | Hs.21486 | M97935 | 8912.27 | 22688.41 | 0.392811572 | 7.83E−08 |
| 3_at | activator of transcription | | | | | | |
| | 1, 91 kD | | | | | | |
| 202954_at | ubiquitin-conjugating | Hs.93002 | NM_007019.1 | 3982.35 | 10133.97 | 0.392970376 | 1.13E−06 |
| | enzyme E2C | | | | | | |
| 209945_{—} | glycogen synthase | Hs.78802 | BC000251.1 | 2414.33 | 6121.16 | 0.394423606 | 4.26E−08 |
| s_at | kinase 3 beta | | | | | | |
| 213553_{—} | apolipoprotein C-I | Hs.268571 | W79394 | 6342.73 | 15981.27 | 0.396885229 | 6.13E−06 |
| x_at | | | | | | | |
| 210004_at | oxidised low density | Hs.77729 | AF035776.1 | 929.49 | 2322.52 | 0.400207533 | 9.33E−06 |
| | lipoprotein (lectin-like) | | | | | | |
| | receptor 1 | | | | | | |
| 208091_{—} | hypothetical protein | Hs.4750 | NM_030796.1 | 7908.33 | 19735.4 | 0.400717999 | 4.32E−09 |
| s_at | DKFZp564K0822 | | | | | | |

Upregulated in normals

| | | | | Normal_{—} | Ttumor_{—} | Fold change | |
|---|---|---|---|---|---|---|---|
| Gene Name | Gene Description | UniGene | GeneBank | median | median | (nor [symbol] | P-value |
| 202037_{—} | secreted frizzled-related | Hs.7306 | NM_003012.2 | 59365.66 | 5359.35 | 11.07702613 | 7.16E−11 |
| s_at | protein 1 | | | | | | |
| 212730_at | KIAA0353 protein | Hs.10587 | AK026420.1 | 46331.26 | 4401.76 | 10.52562157 | 1.72E−12 |
| 205051_{—} | v-kit Hardy-Zuckerman 4 | Hs.81665 | NM_000222.1 | 30870.31 | 3453.96 | 8.937657066 | 1.28E−11 |
| s_at | feline sarcoma viral | | | | | | |
| | oncogene homolog | | | | | | |
| 203881_{—} | dystrophin (muscular | Hs.169470 | NM_004010.1 | 9702.27 | 1267.79 | 7.652899928 | 5.88E−17 |
| s_at | dystrophy, Duchenne and | | | | | | |
| | Becker types) | | | | | | |
| 209292_at | inhibitor of DNA binding | Hs.34853 | NM_001546.1 | 6037.09 | 864.39 | 6.984220086 | 8.13E−11 |
| | 4, dominant negative | | | | | | |
| | helix-loop-helix protein | | | | | | |
| 209291_at | inhibitor of DNA binding | Hs.34853 | NM_001546.1 | 19487.35 | 2908.02 | 6.701243458 | 7.26E−09 |
| | 4, dominant negative | | | | | | |
| | helix-loop-helix protein | | | | | | |
| 202035_{—} | secreted frizzled-related | Hs.7306 | AI332407 | 8226.47 | 1233.99 | 6.666581317 | 1.2E−05 |
| s_at | protein 1 | | | | | | |
| 206825_at | oxytocin receptor | Hs.2820 | NM_000916.2 | 14315.07 | 2188.79 | 6.540175165 | 2.48E−15 |
| 218706_{—} | hypothetical protein | Hs.235445 | AW575493 | 15578.77 | 2719.59 | 5.728352435 | 1.21E−13 |
| s_at | FLJ21313 | | | | | | |
| 202350_{—} | matrilin 2 | Hs.19368 | NM_002380.2 | 11301.25 | 2099.9 | 5.381803895 | 2.25E−07 |
| s_at | | | | | | | |
| 211737_{—} | pleiotrophin (heparin | Hs.44 | BC005916.1 | 19118.74 | 3681.29 | 5.193489239 | 1.98E−09 |
| x_at | binding growth factor 8, | | | | | | |
| | neurite growth-promoting | | | | | | |
| | factor 1) | | | | | | |
| 209863_{—} | tumor protein p63 | Hs.137569 | AF091627.1 | 15557.74 | 3073.13 | 5.062506305 | 5.23E−12 |
| s_at | | | | | | | |
| 218087_{—} | SH3-domain protein 5 | Hs.108924 | NM_015385.1 | 7983.63 | 1692.15 | 4.718039181 | 1.17E−12 |
| s_at | (ponsin) | | | | | | |
| 219795_at | solute carrier family 6 | Hs.162211 | NM_007231.1 | 3443.96 | 767.46 | 4.487478175 | 3.52E−06 |
| | (neuro-transmitter | | | | | | |
| | transporter), member 14 | | | | | | |
| 202342_{—} | tripartite motif- | Hs.12372 | NM_015271.1 | 8892.84 | 2088.2 | 4.258615075 | 5.46E−07 |
| s_at | containing 2 | | | | | | |
| 209290_{—} | nuclear factor I/B | Hs.33287 | BC001283.1 | 51664.48 | 12407.42 | 4.16399864 | 3.45E−06 |
| s_at | | | | | | | |
| 213029_at | Homo sapiens mRNA; cDNA | Hs.326416 | AL110126.1 | 31908.67 | 7680.26 | 4.154634088 | 1.19E−10 |
| | DKFZp564H1916 (from | | | | | | |
| | clone DKFZp564H1916) | | | | | | |
| 203706_{—} | frizzled homolog 7 | Hs.173859 | NM_003507.1 | 19052.38 | 4610.75 | 4.132165049 | 3.3E−07 |
| s_at | (Drosophila) | | | | | | |
| 209392_at | ectonucleotide | Hs.174185 | L35594.1 | 12733.37 | 3091.99 | 4.118179554 | 9.92E−10 |
| | pyrophosphatase/ | | | | | | |
| | phosphodiesterase | | | | | | |
| | 2 (autotaxin) | | | | | | |
| 214598_at | claudin 8 | Hs.162209 | AL049977.1 | 8208.2 | 1993.78 | 4.11690357 | 7.3E−07 |
| 203065_{—} | caveolin 1, caveolae | Hs.74034 | NM_001753.2 | 15611.14 | 3827.36 | 4.078827181 | 1.67E−12 |
| s_at | protein, 22 kD | | | | | | |
| 204731_at | transforming growth | Hs.342874 | NM_003243.1 | 12204.26 | 3072.8 | 3.971706587 | 5.14E−06 |
| | factor, beta receptor | | | | | | |
| | III (betaglycan, 300 kD) | | | | | | |
| 218330_{—} | retinoic acid inducible | Hs.23467 | NM_018162.1 | 12668.28 | 3289.49 | 3.851138018 | 2.24E−08 |
| s_at | in neuroblastoma | | | | | | |
| 203323_at | caveolin 2 | Hs.139851 | BF197655 | 11789.6 | 3069.88 | 3.8404107 | 1E−15 |
| 218804_at | hypothetical protein | Hs.26176 | NM_018043.1 | 12822.63 | 3377.19 | 3.796834054 | 1.74E−06 |
| | FLJ10261 | | | | | | |
| 206481_{—} | LIM domain binding 2 | Hs.4980 | NM_001290.1 | 7116.81 | 1895.62 | 3.754344225 | 1.03E−09 |
| s_at | | | | | | | |
| 208370_{—} | Down syndrome critical | Hs.184222 | NM_004414.2 | 21019.72 | 5602.52 | 3.751833104 | 7.5E−07 |
| s_at | region gene 1 | | | | | | |
| 211726_{—} | flavin containing | Hs.132821 | BC005894.1 | 17812.59 | 4796.43 | 3.713718328 | 3.49E−08 |
| s_at | monooxygenase 2 | | | | | | |
| 201012_at | annexin A1 | Hs.78225 | NM_000700.1 | 41241.85 | 11106.89 | 3.713177136 | 3.91E−10 |
| 212097_at | caveolin 1, caveolae | Hs.74034 | AU147399 | 23596.76 | 6367.19 | 3.705992753 | 3.08E−15 |
| | protein, 22 kD | | | | | | |
| 209170_{—} | glycoprotein M6B | Hs.5422 | AF016004.1 | 8790.1 | 2373.92 | 3.702778527 | 2.01E−07 |
| s_at | aldo-keto reductase | | | | | | |
| | family 1, member C3 | | | | | | |
| | (3-alpha hydroxysteroid | | | | | | |
| 209160_at | dehydrogenase, type II) | Hs.78183 | AB018580.1 | 6068.7 | 1643.09 | 3.693467795 | 2.12E−07 |
| 202746_at | Integral membrane protein | Hs.17109 | AL021786 | 14250.79 | 3939.27 | 3.617622047 | 2.69E−10 |
| | 2A | | | | | | |
| 209894_at | leptin receptor | Hs.226627 | U50748.1 | 3660.94 | 1016.43 | 3.601763033 | 5.5E−11 |
| 203324_{—} | caveolin 2 | Hs.139851 | NM_001233.1 | 6068.91 | 1715.26 | 3.538186631 | 2.97E−10 |
| s_at | | | | | | | |
| 204719_at | ATP-binding cassette, | Hs.38095 | NM_007168.1 | 4833.57 | 1388.04 | 3.482298781 | 5.56E−08 |
| | sub-family A (ABC1), | | | | | | |
| | member 8 | | | | | | |
| 203549_{—} | lipoprotein lipase | Hs.180878 | NM_000237.1 | 10789.01 | 3131.46 | 3.44536095 | 9.05E−11 |
| s_at | | | | | | | |
| 206115_at | early growth response 3 | Hs.74088 | NM_004430.1 | 12017.1 | 3516.09 | 3.41774528 | 5.81E−06 |
| 219935_at | a disintegrin-like and | Hs.58324 | NM_007038.1 | 8376.24 | 2753.5 | 3.405207917 | 3.35E−12 |
| | metalloprotease | | | | | | |
| | (reprolysin type) with | | | | | | |
| | thrombospondin type 1 | | | | | | |
| | motif, 5 (aggrecanase-2) | | | | | | |
| 201656_at | integrin, alpha 6 | Hs.227730 | NM_000210.1 | 9626.26 | 2893.95 | 3.326339432 | 4.04E−07 |
| 205463_{—} | platelet-derived growth | Hs.37040 | NM_002607.1 | 8648.24 | 2619.44 | 3.301560639 | 3.12E−12 |
| s_at | factor alpha polypeptide | | | | | | |
| 823_at | small inducible cytokine | Hs.80420 | U84487 | 12990.21 | 3946.33 | 3.291719142 | 8.6E−07 |
| | subfamily D (Cys-X3-Cys), | | | | | | |
| | member 1 (fractalkine, | | | | | | |
| | neurotactin) | | | | | | |
| 213032_at | Homo sapiens mRNA; cDNA | Hs.326416 | AL110126.1 | 12729.9 | 3880.97 | 3.280082041 | 8.56E−06 |
| | DKFZp564H1916 (from | | | | | | |
| | clone DKFZp564H1916) | | | | | | |
| 217047_{—} | KIAA0914 gene product | Hs.177664 | AK027138.1 | 9278.12 | 2871.79 | 3.230779409 | 5.28E−09 |
| s_at | | | | | | | |
| 209465_{—} | pleiotrophin (heparin | Hs.44 | AL565812 | 7512.2 | 2334.46 | 3.217960471 | 7.53E−08 |
| x_at | binding growth factor 8, | | | | | | |
| | neurite growth-promoting | | | | | | |
| | factor 1) | | | | | | |
| 207808_{—} | protein S (alpha) | Hs.64016 | NM_000313.1 | 5027.75 | 1573.15 | 3.195976226 | 1.7E−09 |
| s_at | | | | | | | |
| 209289_at | nuclear factor I/B | Hs.33287 | AI700518 | 43037.8 | 13478.56 | 3.193056232 | 3.62E−06 |
| 209185_{—} | insulin receptor | Hs.143648 | AF073310.1 | 19990.69 | 6334.2 | 3.155992864 | 1.39E−06 |
| s_at | substrate 2 | | | | | | |
| 202552_{—} | cysteine-rich motor | Hs.19280 | NM_016441.1 | 8386.55 | 2721.46 | 3.081636328 | 8.31E−09 |
| s_at | neuron 1 | | | | | | |
| 203688_at | polycystic kidney | Hs.82001 | NM_000297.1 | 7543.97 | 2462.41 | 3.063653088 | 3.73E−10 |
| | disease 2 (autosomal | | | | | | |
| | dominant) | | | | | | |
| 222162_{—} | a disintegrin-like and | Hs.8230 | AK023795.1 | 10496.22 | 3485.94 | 3.01101568 | 3.81E−06 |
| s_at | metalloprotease | | | | | | |
| | (reprolysin type) with | | | | | | |
| | thrombospondin type 1 | | | | | | |
| | motif, 1 | | | | | | |
| 211685_{—} | neurocalcin delta | Hs.90063 | AF251061.1 | 9352.32 | 3133.91 | 2.984233753 | 1.78E−08 |
| s_at | | | | | | | |
| 213900_at | Friedreich ataxia region | Hs.77889 | AA524029 | 11954.68 | 4037.3 | 2.961058133 | 1.26E−11 |
| | gene X123 | | | | | | |
| 222372_at | ESTs Weakly similar to | Hs.291289 | AW971248 | 8049.26 | 2718.48 | 2.960941408 | 4.62E−06 |
| | ALU1_HUMAN ALU SUBFAMILY | | | | | | |
| | J SEQUENCE CONTAMINATION | | | | | | |
| | WARNING ENTRY [H. sapiens] | | | | | | |
| 201540_at | four and a half LIM | Hs.239069 | NM_001449.1 | 17627.89 | 6015.25 | 2.930533228 | 4.28E−08 |
| | domains 1 | | | | | | |
| 212254_{—} | bullous pemphigoid | Hs.198689 | BG253119 | 19972.78 | 6991.03 | 2.856915219 | 1.32E−09 |
| s_at | antigen 1 (230/240 kD) | | | | | | |
| 213353_at | ATP-binding cassette, | Hs.180513 | BF693921 | 5730.62 | 2019.34 | 2.837867818 | 3.71E−10 |
| | sub-family A (ABC1), | | | | | | |
| | member 5 | | | | | | |
| 205498_at | growth hormone receptor | Hs.125180 | NM_000163.1 | 7384.79 | 2603.42 | 2.836572662 | 4.63E−06 |
| 215016_{—} | bullous pemphigoid | Hs.198689 | BC004912.1 | 19089.82 | 6747.39 | 2.829215445 | 3.72E−09 |
| x_at | antigen 1 (230/240 kD) | | | | | | |
| 208944_at | transforming growth | Hs.82028 | D50683.1 | 18938.86 | 6698.52 | 2.827320065 | 7.59E−12 |
| | factor, beta receptor | | | | | | |
| | II (70-80 kD) | | | | | | |
| 210839_{—} | ectonucleotide | Hs.174185 | D45421.1 | 7024.74 | 2493.07 | 2.817706683 | 4.26E−13 |
| s_at | pyrophosphatase/ | | | | | | |
| | phosphodiesterase | | | | | | |
| | 2 (autotaxin) | | | | | | |
| 218901_at | phospholipid scramblase | Hs.182538 | NM_020353.1 | 8923.62 | 3169.64 | 2.815341805 | 1.56E−10 |
| | 4 | | | | | | |
| 209466_{—} | pleiotrophin (neparin | Hs.44 | M57399.1 | 18099.82 | 6464.73 | 2.799779728 | 4.27E−08 |
| x_at | binding growth factor 8, | | | | | | |
| | neurite growth-promoting | | | | | | |
| | factor 1) | | | | | | |
| 200795_at | SPARC-like 1 (mast9, | Hs.75445 | NM_004684.1 | 62309.15 | 22325.59 | 2.790929601 | 4.78E−07 |
| | hevin) | | | | | | |
| 202973_{—} | KIAA0914 gene | Hs.177664 | NM_014883.1 | 11301.89 | 4053.46 | 2.788208099 | 4.1E−07 |
| x_at | product | | | | | | |
| 218723_{—} | RGC32 protein | Hs.76640 | NM_014059.1 | 13133.05 | 4722.25 | 2.781100111 | 2.13E−07 |
| s_at | | | | | | | |
| 213375_{—} | hypothetical gene | Hs.22174 | N80918 | 9894.2 | 3571.88 | 2.770025869 | 2.77E−09 |
| s_at | CG018 | | | | | | |
| 221841_{—} | Kruppel-like factor | Hs.356370 | BF514078 | 17464.66 | 6347.92 | 2.751241351 | 1.3E−06 |
| s_at | 4 (gut) | | | | | | |
| 218276_{—} | WW45 protein | Hs.288906 | NM_021818.1 | 6994.97 | 2552.32 | 2.740832052 | 4.14E−09 |
| s_at | | | | | | | |
| 212463_at | Homo sapiens mRNA; cDNA | Hs.99766 | BE379006 | 23386.73 | 8711.13 | 2.684695327 | 2.02E−08 |
| | DKFZp564J0323 (from | | | | | | |
| | clone DKFZp564J0323) | | | | | | |
| 213486_at | hypothetical protein | Hs.6421 | BF435376 | 4412.93 | 1649.6 | 2.675151552 | 2.78E−14 |
| | DKFZp761N09121 | | | | | | |
| 206306_at | ryanodine receptor 3 | Hs.9349 | NM_001036.1 | 2449.43 | 926.73 | 2.643089141 | 3.38E−09 |
| 212675_{—} | KIAA0582 protein | Hs.79507 | AB011154.1 | 6645.48 | 2532.1 | 2.624493503 | 4.88E−12 |
| s_at | | | | | | | |
| 200762_at | dihydropyrimidinase- | Hs.173381 | NM_001386.1 | 24509.97 | 9355.96 | 2.619717271 | 1.4E−08 |
| | like 2 | | | | | | |
| 207480_{—} | Meis1, myeloid ecotropic | Hs.104105 | NM_020149.1 | 5180.76 | 2010.23 | 2.577197634 | 2.37E−07 |
| s_at | viral integration site 1 | | | | | | |
| | homolog 2 (mouse) | | | | | | |
| 219091_{—} | EMILIN-like protein | Hs.127216 | NM_024756.1 | 6277.33 | 2442.04 | 2.5705271 | 4.58E−13 |
| s_at | EndoGlyx-1 | | | | | | |
| 219304_{—} | spinal cord-derived | Hs.112885 | NM_025208.1 | 10905.82 | 4319.06 | 2.525044801 | 9.33E−10 |
| s_at | growth factor-B | | | | | | |
| 207542_{—} | aquaporin 1 (channel- | Hs.74602 | NM_000385.2 | 8557.32 | 3405.56 | 2.512749739 | 8.69E−07 |
| s_at | forming integral | | | | | | |
| | protein, 28 kD) | | | | | | |
| 211998_at | H3 histone, family 38 | Hs.180877 | NM_005324.1 | 10030.86 | 3995.83 | 2.510332021 | 8.65E−06 |
| | (H3.3B) | | | | | | |
| 204115_at | guanine nucleotide | Hs.83381 | NM_004126.1 | 5852.14 | 2337.15 | 2.50396423 | 2.41E−07 |
| | binding protein 11 | | | | | | |
| 202016_at | mesoderm specific | Hs.70284 | NM_002402.1 | 21998.29 | 8805.67 | 2.498196049 | 1.05E−07 |
| | transcript homolog (mouse) | | | | | | |

Probe = Affymetrix Probe Sequence
Description = Gene name and annotation
Unigene = Unigene Number (NCBI)
Genbank = Genbank Accession Number
Median = Median expression value in Normals or Tumors
Fold change = Ratio of expression values (normals/tumors)
P-value = t-test significance

TABLE 4b
Minimal Geneset for the Classification of Normal vs Tumor

| | | | |
|---|---|---|---|
| Probe | Gene Description | UniGene | GeneBank |

Upregulated in Tumors

| | | | |
|---|---|---|---|
| 201954_at | actin related protein ⅔ complex, subunit 1B (41 kD) | Hs.11538 | NM_005720.1 |
| 213905_x_at | biglycan | Hs.821 | AA845258 |
| 201261_x_at | biglycan | Hs.821 | BC002416.1 |
| 202391_at | brain abundant, membrane attached signal protein 1 | Hs.79516 | NM_006317.1 |
| 205483_s_at | interferon-stimulated protein, 15 kDa | Hs.833 | NM_005101.1 |
| 221729_at | collagen, type V, alpha 2 | Hs.82985 | NM_000393.1 |
| 211161_s_at | collagen, type III, alpha 1 (Ehlers-Danlos syndrome type IV, | Hs.119571 | AF130082.1 |
| | autosomal dominant) | | |
| 201422_at | interferon, gamma-inducible protein 30 | Hs.14623 | NM_008332.1 |
| 203936_s_at | matrix metalloproteinase 9 (gelatinase B, 92 kD gelatinase, | Hs.151738 | NM_004994.1 |
| | 92 kD type IV collagenase) | | |
| 210004_at | oxidised low density lipoprotein (lectin-like) receptor 1 | Hs.77729 | AF035776.1 |
| 208998_at | uncoupling protein 2 (mitochondrial, proton carrier) | Hs.80658 | U94592.1 |
| 222039_at | hypothetical protein FLJ11029 | Hs.274448 | AA292789 |

Upregulated in Normals

| | | | |
|---|---|---|---|
| 209160_at | aldo-keto reductase family 1, member C3 (3-alpha | Hs.78183 | AB018580.1 |
| | hydroxysteroid dehydrogenase, type II) | | |
| 201012_at | annexin A1 | Hs.78225 | NM_000700.1 |
| 204719_at | ATP-binding cassette, sub-family A (ABC1), member 8 | Hs.38095 | NM_007168.1 |
| 221841_s_at | Kruppel-like factor 4 (gut) | Hs.356370 | BF514079 |
| 210839_s_at | ectonucleotide pyrophosphatase/phosphodiesterase 2 | Hs.174185 | D45421.1 |
| | (autotaxin) | | |
| 209392_at | ectonucleotide pyrophosphatase/phosphodiesterase 2 | Hs.174185 | L35594.1 |
| | (autotaxin) | | |
| 201540_at | four and a half LIM domains 1 | Hs.239069 | NM_001449.1 |
| 202342_s_at | tripartite motif-containing 2 | Hs.12372 | NM_015271.1 |
| 209185_s_at | insulin receptor substrate 2 | Hs.143648 | AF073310.1 |
| 209894_at | leptin receptor | Hs.226627 | U50748.1 |
| 206481_s_at | LIM domain binding 2 | Hs.4980 | NM_001290.1 |
| 202016_at | mesoderm specific transcript homolog (mouse) | Hs.79284 | NM_002402.1 |
| 209290_s_at | nuclear factor I/B | Hs.33287 | BC001283.1 |
| 218901_at | phospholipid scramblase 4 | Hs.182538 | NM_020353.1 |
| 209466_x_at | pleiotrophin (heparin binding growth factor 8, | Hs.44 | M57399.1 |
| | neurite growth-promoting factor 1) | | |
| 211737_x_at | pleiotrophin (heparin binding growth factor 8, | Hs.44 | BC005916.1 |
| | neurite growth-promoting factor 1) | | |
| 202037_s_at | secreted frizzled-related protein 1 | Hs.7306 | NM_003012.2 |
| 205051_s_at | v-kit Hardy-Zuckerman 4 feline sarcoma viral oncogene | Hs.81665 | NM_000222.1 |
| | homolog | | |
| 212730_at | KIAA0353 protein | Hs.10587 | AK026420.1 |
| 218330_s_at | retinoic acid inducible in neuroblastoma | Hs.23467 | NM_018162.1 |

TABLE 5A
CGS for ER and ERBB2 Classification
ER Classification Genes

| Probe | Gene Name | Unigene | Gen Bank | Regulation |
|---|---|---|---|---|
| 205225_at | estrogen receptor 1 | Hs.1657 | NM_000125.1 | + |
| 203963_at | carbonic anhydrase XII | Hs.5338 | NM_001218.2 | + |
| 209602_s_at | GATA binding protein 3 | Hs.169946 | AI796169 | + |
| 214164_x_at | adaptor-related protein complex 1, gamma 1 subunit | Hs.5344 | BF752277 | + |
| 202089_s_at | LIV-1 protein, estrogen regulated | Hs.79136 | NM_012319.2 | + |
| 212956_at | KIAA0882 protein | Hs.90419 | AB020689.1 | + |
| 214440_at | N-acetyltransferase 1 (arylamine N-acetyltransferase) | Hs.165956 | NM_000662.1 | + |
| 206754_s_at | cytochrome P450, subfamily IIB (phenobarbital-inducible), | Hs.1360 | NM_000767.2 | + |
| | polypeptide 6 | | | |
| 222212_s_at | LAG1 longevity assurance homolog 2 (S. cerevisiae) | Hs.285976 | AK001105.1 | + |
| 218195_at | hypothetical protein FLJ12910 | Hs.15929 | NM_024573.1 | + |
| 205862_at | KIAA0575 gene product | Hs.193914 | NM_014668.1 | + |
| 212195_at | Homo sapiens mRNA; cDNA DKFZp564F053 (from | Hs.71968 | AL049265.1 | + |
| | clone DKFZp564F053) | | | |
| 208682_s_at | melanoma antigen, family D, 2 | Hs.4943 | AF126181.1 | + |
| 202342_s_at | tripartite motif-containing 2 | Hs.12372 | NM_015271.1 | − |
| 209459_s_at | NPD009 protein | Hs.283675 | AF237813.1 | + |
| 201037_at | phosphofructokinase, platelet | Hs.99910 | NM_002627.1 | − |
| 203571_s_at | adipose specific 2 | Hs.74120 | NM_006829.1 | + |
| 214088_s_at | fucosyltransferase 3 (galactoside 3(4)-L-fucosyltransferase, | Hs.169238 | AW080549 | − |
| | Lewis blood group included) | | | |
| 201976_s_at | myosin X | Hs.61638 | NM_012334.1 | − |
| 218502_s_at | trichorhinophalangeal syndrome I | Hs.26102 | NM_014112.1 | + |
| 203221_at | transducin-like enhancer of split 1 (E(sp1) homolog, | Hs.28935 | AI951720 | − |
| | Drosophila) | | | |
| 207002_s_at | pleiomorphic adenoma gene-like 1 | Hs.75825 | NM_002656.1 | − |
| 207030_s_at | cysteine and glycine-rich protein 2 | Hs.10526 | NM_001321.1 | − |
| 204623_at | trefoil factor 3 (intestinal) | Hs.352107 | NM_003226.1 | + |
| 205009_at | trefoil factor 1 (breast cancer, estrogen-inducible | Hs.350470 | NM_003225.1 | + |
| | sequence expressed in) | | | |

Regulation = On (+) or Off (−) in an ER+ tumor

TABLE 5B
ERBB2 Classification Genes

| Probe | Gene Name | Unigene | GenBank | Regulation |
|---|---|---|---|---|
| 216836_s_at | v-erb-b2 erythroblastic leukemia viral oncogene homolog 2, | Hs.323910 | X03363.1 | + |
| | neuro/glioblastoma derived oncogene homolog (avian) | | | |
| 210761_s_at | growth factor receptor-bound protein 7 | Hs.86859 | AB008790.1 | + |
| 202991_at | steroidogenic acute regulatory protein related | Hs.77628 | NM_006804.1 | + |
| 55616_at | hypothetical gene MGC9753 | Hs.91668 | AI703342 | + |
| 214203_s_at | proline dehydrogenase (oxidase) 1 | Hs.343874 | AA074145 | + |
| 213557_at | KIAA0904 protein | Hs.278346 | AW305119 | + |
| 220149_at | hypothetical protein FLJ22671 | Hs.193745 | NM_024861.1 | + |
| 215659_at | Homo sapiens cDNA: FLJ21521 fis, clone COL0588O | Hs.306777 | AK025174.1 | + |
| 219233_s_at | hypothetical protein PRO2521 | Hs.19054 | NM_018530.1 | + |
| 203497_at | PPAR binding protein | Hs.15589 | NM_004774.1 | + |
| 219226_at | CDC2-related protein kinase 7 | Hs.123073 | NM_016507.1 | + |
| 202712_s_at | creatine kinase, mitochondrial 1 (ubiquitous) | Hs.153998 | NM_020990.2 | + |
| 204285_s_at | phorbol-12-myristate-13-acetate-induced protein 1 | Hs.96 | AI857639 | − |
| 205225_at | estrogen receptor 1 | Hs.1657 | NM_000125.1 | − |
| 214614_at | homeo box HB9 | Hs.37035 | AI738662 | + |
| 202917_s_at | S100 calcium binding protein A8 (calgranulin A) | Hs.100000 | NM_002964.2 | + |
| 219429_at | fatty acid hydroxylase | Hs.249163 | NM_024306.1 | + |
| 208614_s_at | filamin B, beta (actin binding protein 278) | Hs.81008 | M62994.1 | − |
| 204029_at | cadherin, EGF LAG seven-pass G-type receptor 2 (flamingo | Hs.57652 | NM_001408.1 | − |
| | homolog, Drosophila) | | | |
| 216401_x_at | Homo sapiens partial IGKV gene for Immunoglobulin | Hs.307136 | AJ408433 | + |
| | kappa chain variable region, clone 38 | | | |
| 203685_at | B-cell CLL/lymphoma 2 | Hs.79241 | NM_000633.1 | − |
| 216576_x_at | Homo sapiens isolate donor. N clone N88K | Hs.247910 | AF103529.1 | + |
| | Immunoglobulin kappa light chain variable | | | |
| | region mRNA, partial cds | | | |
| 211138_s_at | kynurenine 3-monooxygenase (kynurenine 3-hydroxylase) | Hs.107318 | BC005297.1 | + |
| 202039_at | TGFB1-induced anti-apoptotic factor 1 | Hs.78822 | NM_004740.1 | + |
| 203627_at | insulin-like growth factor 1 receptor | Hs.239176 | NM_000875.2 | − |
| 204863_s_at | interleukin 6 signal transducer (gp130, oncostatin | Hs.82065 | BE856546 | − |
| | M receptor) | | | |

TABLE 6a
Predictor Sets for Molecular Subtype Using OVA SVM

| Luminal A | | | |
|---|---|---|---|
| Probe | Gene Description | UniGene | GeneBank |
| 201030_x_at | lactate dehydrogenase B | Hs.234489 | NM_002300.1 |
| 201525_at | apolipoprotein D | Hs.75736 | NM_001647.1 |
| 201688_s_at | tumor protein D52 | Hs.2384 | BE974098 |
| 201754_at | cytochrome c oxidase subunit Vic | Hs.351875 | NM_004374.1 |
| 202376_at | serine (or cysteine) proteinase inhibitor, clade A | Hs.234726 | NM_001085.2 |
| | (alpha-1 antiproteinase, antitrypsin), member 3 | | |
| 202555_s_at | myosin, light polypeptide kinase | Hs.211582 | NM_005965.1 |
| 202746_at | Integral membrane protein 2A | Hs.17109 | AL021786 |
| 202991_at | steroidogenic acute regulatory protein related | Hs.77628 | NM_006804.1 |
| 203627_at | insulin-like growth factor 1 receptor | Hs.239176 | NM_000875.2 |
| 203749_s_at | retinoic acid receptor, alpha | Hs.250505 | AI806984 |
| 204198_s_at | runt-related transcription factor 3 | Hs.170019 | AA541630 |
| 204304_s_at | prominin-like 1 (mouse) | Hs.112360 | NM_006017.1 |
| 205225_at | estrogen receptor 1 | Hs.1657 | NM_000125.1 |
| 205471_s_at | dachshund homolog (Drosophila) | Hs.63931 | AW772082 |
| 206378_at | secretoglobin, family 2A, member 2 | Hs.46452 | NM_002411.1 |
| 208711_s_at | cyclin D1 (PRAD1: parathyroid adenomatosis 1) | Hs.82932 | BC000076.1 |
| 209016_s_at | keratin 7 | Hs.23881 | BC002700.1 |
| 209290_s_at | nuclear factor I/B | Hs.33287 | BC001283.1 |
| 209292_at | inhibitor of DNA binding 4, dominant negative | Hs.34853 | NM_001546.1 |
| | helix-loop-helix protein | | |
| 209351_at | keratin 14 (epidermolysis bullosa simplex, | Hs.117729 | BC002690.1 |
| | Dowling-Meara, Koebner) | | |
| 209398_s_at | chitinase 3-like 1 (cartilage glycoprotein-39) | Hs.75184 | M80927.1 |
| 209465_x_at | pleiotrophin (heparin binding growth factor 8, | Hs.44 | AL565812 |
| | neurite growth-promoting factor 1) | | |
| 209863_s_at | tumor protein p63 | Hs.137569 | AF091627.1 |
| 211538_s_at | heat shock 70 kD protein 2 | Hs.75452 | U56725.1 |
| 211726_s_at | flavin containing monooxygenase 2 | Hs.132821 | BC005894.1 |
| 211737_x_at | pleiotrophin (heparin binding growth factor 8, | Hs.44 | BC005916.1 |
| | neurite growth-promoting factor 1) | | |
| 211958_at | Homo sapiens, clone IMAGE: 4183312, | Hs.180324 | L27560.1 |
| | mRNA, partial cds | | |
| 211959_at | Homo sapiens, clone IMAGE: 4183312, | Hs.180324 | L27560.1 |
| | mRNA, partial cds | | |
| 212730_at | KIAA0353 protein | Hs.10587 | AK026420.1 |
| 213564_x_at | lactate dehydrogenase B | Hs.234489 | BE042354 |
| 216836_s_at | v-erb-b2 erythroblastic leukemia viral oncogene | Hs.323910 | X03363.1 |
| | homolog 2, neuro/glioblastoma derived oncogene | | |
| | homolog (avian) | | |
| 217762_s_at | RAB31, member RAS oncogene family | Hs.223025 | BE789881 |
| 217838_s_at | RNB6 | Hs.241471 | NM_016337.1 |
| 218532_s_at | hypothetical protein FLJ20152 | Hs.82273 | NM_019000.1 |
| 221765_at | Homo sapiens mRNA full length insert cDNA | Hs.23703 | BF970427 |
| | clone EUROIMAGE 1287006 | | |

ER-Subtype II

| Probe | Gene Description | UniGene | GeneBank |
|---|---|---|---|
| 200099_s_at | Human DNA sequence from clone RP11-486O22 on chromosome 10 | Hs.307132 | AL356115 |
| | Contains the 3part of a gene for KIAA1128 protein, a novel | | |
| | pseudogene, a gene for protein similar to RPS3A (ribosomal | | |
| | protein S3A), ESTs, STSs, GSSs and CpG islands | | |
| 37892_at | collagen, type XI, alpha 1 | Hs.82772 | J04177 |
| 39248_at | aquaporin 3 | Hs.234642 | N74607 |
| 200606_at | desmoplakin (DPI, DPII) | Hs.349499 | NM_004415.1 |
| 200706_s_at | LPS-induced TNF-alpha factor | Hs.76507 | NM_004862.1 |
| 200749_at | RAN, member RAS oncogene family | Hs.10842 | BF112006 |
| 200811_at | cold inducible RNA binding protein | Hs.119475 | NM_001280.1 |
| 200823_x_at | ribosomal protein L29 | Hs.350068 | NM_000992.1 |
| 200853_at | H2A histone family, member Z | Hs.119192 | NM_002106.1 |
| 200925_at | cytochrome c oxidase subunit Via polypeptide 1 | Hs.180714 | NM_004373.1 |
| 200935_at | calreticulin | Hs.16488 | NM_004343.2 |
| 201054_at | heterogeneous nuclear ribonucleoprotein A0 | Hs.77492 | BE966599 |
| 201080_at | phosphatidylinositol-4-phosphate 5-kinase, type II, beta | Hs.6335 | BF338509 |
| 201131_s_at | cadherin 1, type 1, E-cadherin (epithelial) | Hs.194657 | NM_004360.1 |
| 201134_x_at | cytochrome c oxidase subunit Vllc | Hs.3462 | NM_001867.1 |
| 201291_s_at | topoisomerase (DNA) II alpha (170 kD) | Hs.156346 | NM_001067.1 |
| 201349_at | solute carrier family 9 (sodium/hydrogen exchanger), | Hs.184276 | NM_004252.1 |
| | isoform 3 regulatory factor 1 | | |
| 201431_s_at | dihydropyrimidinase-like 3 | Hs.74566 | NM_001387.1 |
| 201552_at | lysosomal-associated membrane protein 1 | Hs.150101 | NM_005561.2 |
| 201688_s_at | tumor protein D52 | Hs.2384 | BE974098 |
| 201689_s_at | tumor protein D52 | Hs.2384 | BE974098 |
| 201830_s_at | neuroepithelial cell transforming gene 1 | Hs.25155 | NM_005863.1 |
| 201890_at | ribonucleotide reductase M2 polypeptide | Hs.75319 | NM_001034.1 |
| 201892_s_at | IMP (inosine monophosphate) dehydrogenase 2 | Hs.75432 | NM_000884.1 |
| 201903_at | ubiquinol-cytochrome c reductase core protein I | Hs.119251 | NM_003365.1 |
| 201925_s_at | decay accelerating factor for complement (CD55, | Hs.1369 | NM_000574.1 |
| | Cromer blood group system) | | |
| 201946_s_at | chaperonin containing TCP1, subunit 2 (beta) | Hs.6456 | AL545982 |
| 202071_at | syndecan 4 (amphiglycan, ryudocan) | Hs.252189 | NM_002999.1 |
| 202088_at | LIV-1 protein, estrogen regulated | Hs.79136 | AI635449 |
| 202291_s_at | matrix Gla protein | Hs.365706 | NM_000900.1 |
| 202376_at | serine (or cysteine) proteinase inhibitor, clade A | Hs.234726 | NM_001085.2 |
| | (alpha-1 antiproteinase, antitrypsin), member 3 | | |
| 202489_s_at | FXYD domain-containing ion transport regulator 3 | Hs.301350 | BC005238.1 |
| 202704_at | transducer of ERBB2, 1 | Hs.178137 | AA675892 |
| 203202_at | HIV-1 rev binding protein 2 | Hs.154762 | AI950314 |
| 203627_at | insulin-like growth factor 1 receptor | Hs.239176 | NM_000875.2 |
| 203628_at | insulin-like growth factor 1 receptor | Hs.239176 | NM_000875.2 |
| 203789_s_at | sema domain, immunoglobulin domain (Ig), short basic | Hs.171921 | NM_006379.1 |
| | domain, secreted, (semaphorin) 3C | | |
| 203892_at | WAP four-disulfide core domain 2 | Hs.2719 | NM_006103.1 |
| 203915_at | monokine induced by gamma interferon | Hs.77367 | NM_002416.1 |
| 203929_s_at | Homo sapiens cDNA FLJ31424 fis, clone NT2NE2000392 | Hs.101174 | NM_016835.1 |
| 203963_at | carbonic anhydrase XII | Hs.5338 | NM_001218.2 |
| 204018_x_at | hemoglobin, alpha 1 | Hs.272572 | NM_000558.2 |
| 204031_s_at | poly(rC) binding protein 2 | Hs.63525 | NM_005016.1 |
| 204320_at | collagen, type XI, alpha 1 | Hs.82772 | NM_001854.1 |
| 204457_s_at | growth arrest-specific 1 | Hs.65029 | NM_002048.1 |
| 205225_at | estrogen receptor 1 | Hs.1657 | NM_000125.1 |
| 205428_s_at | calbindin 2, (29 kD, calretinin) | Hs.106857 | NM_001740.2 |
| 205453_at | homeo box B2 | Hs.2733 | NM_002145.1 |
| 205887_x_at | mutS homolog 3 (E. coli) | Hs.42674 | NM_002439.1 |
| 205941_s_at | collagen, type X, alpha 1(Schmid metaphyseal | Hs.179729 | AI376003 |
| | chondrodysplasia) | | |
| 206211_at | selectin E (endothelial adhesion molecule 1) | Hs.89546 | NM_000450.1 |
| 206916_x_at | tyrosine aminotransferase | Hs.161640 | NM_000353.1 |
| 207721_x_at | histidine triad nucleotide binding protein 1 | Hs.256697 | NM_005340.1 |
| 208702_x_at | amyloid beta (A4) precursor-like protein 2 | Hs.279518 | BC000373.1 |
| 208703_s_at | amyloid beta (A4) precursor-like protein 2 | Hs.279518 | BC000373.1 |
| 208711_s_at | cyclin D1 (PRAD1: parathyroid adenomatosis 1) | Hs.82932 | BC000076.1 |
| 208764_s_at | ATP synthase, H+ transporting, mitochondrial F0 | Hs.89399 | D13119.1 |
| | complex, subunit c (subunit 9), isoform 2 clusterin | | |
| | (complement lysis inhibitor, SP-40, 40, sulfated | | |
| | glycoprotein 2, testosterone-repressed | | |
| | prostate message | | |
| 208791_at | 2, apolipoprotein J) clusterin (complement lysis | Hs.75106 | M25915.1 |
| | inhibitor, SP-40, 40, sulfated glycoprotein 2, | | |
| | testosterone-repressed prostate message | | |
| 208792_s_at | 2, apolipoprotein J) | Hs.75106 | M25915.1 |
| 208826_x_at | histidine triad nucleotide binding protein 1 | Hs.256697 | U27143.1 |
| 208950_s_at | aldehyde dehydrogenase 7 family, member A1 | Hs.74294 | BC002515.1 |
| 209035_at | midkine (neurite growth-promoting factor 2) | Hs.82045 | M69148.1 |
| 209069_s_at | H3 histone, family 3B (H3.3B) | Hs.180877 | BC001124.1 |
| 209112_at | cyclin-dependent kinase inhibitor 1B (p27, Kip1) | Hs.238990 | BC001971.1 |
| 209116_x_at | hemoglobin, beta | Hs.155376 | M25079.1 |
| 209143_s_at | chloride channel, nucleotide-sensitive, 1A | Hs.84974 | AF005422.1 |
| 209351_at | keratin 14 (epidermolysis bullosa simplex, | Hs.117729 | BC002690.1 |
| | Dowling-Meara, Koebner) | | |
| 209369_at | annexin A3 | Hs.1378 | M63310.1 |
| 209403_at | hypothetical protein DKFZp434P2235 | Hs.105891 | AL136860.1 |
| 209602_s_at | GATA binding protein 3 | Hs.169946 | AI796169 |
| 210163_at | small inducible cytokine subfamily B (Cys-X-Cys), | Hs.103982 | AF030514.1 |
| | member 11 | | |
| 210387_at | H2B histone family, member A | Hs.352109 | BC001131.1 |
| 210511_s_at | inhibin, beta A (activin A, activin AB alpha | Hs.727 | M13436.1 |
| | polypeptide) | | |
| 210715_s_at | serine protease inhibitor, Kunitz type, 2 | Hs.31439 | AF027205.1 |
| 210764_s_at | cysteine-rich, angiogenic inducer, 61 | Hs.8867 | AF003114.1 |
| 211113_s_at | ATP-binding cassette, sub-family G (WHITE), | Hs.10237 | U34919.1 |
| | member 1 | | |
| 211404_s_at | amyloid beta (A4) precursor-like protein 2 | Hs.279518 | BC004371.1 |
| 211696_x_at | hemoglobin, beta | Hs.155376 | AF349114.1 |
| 211745_x_at | hemoglobin, alpha 2 | Hs.347939 | BC005931.1 |
| 211935_at | ADP-ribosylation factor-like 6 interacting protein | Hs.75249 | D31885.1 |
| 212328_at | KIAA1102 protein | Hs.202949 | AK027231.1 |
| 212492_s_at | KIAA0876 protein | Hs.301011 | AW237172 |
| 212692_s_at | vesicle trafficking, beach and anchor containing | Hs.62354 | W60686 |
| 212942_s_at | KIAA1199 protein | Hs.50081 | AB033025.1 |
| 212956_at | KIAA0882 protein | Hs.90419 | AB020689.1 |
| 3213557_at | KIAA0904 protein | Hs.278346 | AW305119 |
| 213764_s_at | Microfibril-associated glycoprotein-2 | Hs.300946 | AW665892 |
| 213765_at | Microfibril-associated glycoprotein-2 | Hs.300946 | AW665892 |
| 214079_at | Homo sapiens cDNA FLJ20338 fis, clone HEP12179 | Hs.152677 | AK000345.1 |
| 214414_x_at | hemoglobin, alpha 2 | Hs.347939 | T50399 |
| 214836_x_at | immunoglobulin kappa constant | Hs.156110 | BG536224 |
| 215224_at | Homo sapiens cDNA: FLJ21547 fis, clone COL06206 | Hs.322680 | AK025200.1 |
| 215867_x_at | adaptor-related protein complex 1, gamma 1 subunit | Hs.5344 | AL050025.1 |
| 217014_s_at | Homo sapiens PAC clone RP4-604G5 from 7q22-q31.1 | Hs.307354 | AC004522 |
| 217428_s__at | collagen, type X, alpha 1 (Schmid metaphyseal | Hs.179729 | X98568 |
| | chondrodysplasia) ESTs, Moderately similar to | | |
| | ALU7_HUMAN ALU SUBFAMILY SQ SEQUENCE | | |
| | CONTAMINATION WARNING | | |
| 217704_x_at | ENTRY [H. sapiens] | Hs.310806 | AI820796 |
| 217753_s_at | ribosomal protein S26 | Hs.299465 | NM_001029.1 |
| 218237_s_at | solute carrier family 38, member 1 | Hs.18272 | NM_030674.1 |
| 218302_at | uncharacterized hematopoietic stem/progenitor | Hs.54960 | NM_018468.1 |
| | cells protein MDS033 | | |
| 218388_at | 6-phosphogluconolactonase | Hs.100071 | NM_012088.1 |
| 218468_s_at | cysteine knot superfamily 1, BMP antagonist 1 | Hs.40098 | AF154054.1 |
| 218469_at | cysteine knot superfamily 1, BMP antagonist 1 | Hs.40098 | NM_013372.1 |
| 219087_at | asporin (LRR class 1) | Hs.10760 | NM_017680.1 |
| 219454_at | EGF-like-domain, multiple 6 | Hs.12844 | NM_015507.2 |
| 219734_at | hypothetical protein FLJ20174 | Hs.114556 | NM_017699.1 |
| 219773_at | NADPH oxidase 4 | Hs.93847 | NM_016931.1 |
| 220149_at | hypothetical protein FLJ22671 | Hs.193745 | NM_024861.1 |
| 220864_s_at | cell death-regulatory protein GRIM19 | Hs.279574 | NM_015965.1 |
| 221434_s_at | hypothetical protein DC50 | Hs.324521 | NM_031210.1 |
| 221473_x_at | tumor differentially expressed 1 | Hs.272168 | U49188.1 |
| 221541_at | hypothetical protein DKFZp434B044 | Hs.262958 | AL136861.1 |

Basal

| | | | |
|---|---|---|---|
| 202342_s_at | tripartite motif-containing 2 | Hs.12372 | NM_015271.1 |
| 202345_s_at | fatty acid binding protein 5 (psoriasis-associated) | Hs.153179 | NM_001444.1 |
| 202412_s_at | ubiquitin specific protease 1 | Hs.35086 | AW499935 |
| 203780_at | epithelial V-like antigen 1 | Hs.116851 | AF275945.1 |
| 204580_at | matrix metalloproteinase 12 (macrophage elastase) | Hs.1695 | NM_002426.1 |
| 205066_s_at | ectonucleotide pyrophosphatase/phosphodiesterase 1 | Hs.11951 | NM_006208.1 |
| 206042_x_at | SNRPN upstream reading frame | Hs.58606 | NM_022804.1 |
| 206102_at | KIAA0186 gene product | Hs.36232 | NM_021067.1 |
| 209205_s_at | LIM domain only 4 | Hs.3844 | BC003600.1 |
| 209212_s_at | Kruppel-like factor 5 (intestinal) | Hs.84728 | AB030824.1 |
| 209351_at | keratin 14 (epidermolysis bullosa simplex, | Hs.117729 | BC002690.1 |
| | Dowling-Meara, Koebner) | | |
| 212236_x_at | keratin 17 | Hs.2785 | Z19574 |
| 212592_at | Homo sapiens, clone MGC: 24130 IMAGE: 4692359, | Hs.76325 | AV733266 |
| | mRNA, complete cds | | |
| 213664_at | solute carrier family 1 (neuronal/epithelial high | Hs.91139 | AW235061 |
| | affinity glutamate transporter, system Xag), member 1 | | |
| 213668_s_at | SRY (sex determining region Y)-box 4 | Hs.83484 | AI989477 |
| 213680_at | keratin 6B | Hs.335952 | AI831452 |
| 217744_s_at | p53-induced protein PIGPC1 | Hs.303125 | NM_022121.1 |
| 218499_at | Mst3 and SOK1-related kinase | Hs.23643 | NM_016542.1 |
| 218593_at | hypothetical protein FLJ10377 | Hs.274263 | NM_018077.1 |
| 222039_at | hypothetical protein FLJ11029 | Hs.274448 | AA292789 |

ERBB2

| | | | |
|---|---|---|---|
| 55616_at | hypothetical gene MGC9753 | Hs.91668 | AI703342 |
| 201388_at | proteasome (prosome, macropain) 26S subunit, non- | Hs.9736 | NM_002809.1 |
| | ATPase, 3 | | |
| 201525_at | apolipoprotein D | Hs.75736 | NM_001647.1 |
| 202035_s_at | secreted frizzled-related protein 1 | Hs.7306 | AI332407 |
| 202036_s_at | secreted frizzled-related protein 1 | Hs.7306 | AF017987.1 |
| 202145_at | lymphocyte antigen 6 complex, locus E | Hs.77667 | NM_002346.1 |
| 202218_s_at | fatty acid desaturase 2 | Hs.184641 | NM_004265.1 |
| 202376_at | serine (or cysteine) proteinase inhibitor, clade A | Hs.234726 | NM_001085.2 |
| | (alpha-1 antiproteinase, antitrypsin), member 3 | | |
| 202991_at | steroidogenic acute regulatory protein related | Hs.77628 | NM_006804.1 |
| 203355_s_at | KIAA0942 protein | Hs.6763 | NM_015310.1 |
| 203404_at | armadillo repeat protein ALEX2 | Hs.48924 | NM_014782.1 |
| 203439_s_at | stanniocalcin 2 | Hs.155223 | BC000658.1 |
| 203628_at | insulin-like growth factor 1 receptor | Hs.239176 | NM_000875.2 |
| 203685_at | B-cell CLL/lymphoma 2 | Hs.79241 | NM_000633.1 |
| 204734_at | keratin 15 | Hs.80342 | NM_002275.1 |
| 204942_s_at | aldehyde dehydrogenase 3 family, member B2 | Hs.87539 | NM_000695.2 |
| 205225_at | estrogen receptor 1 | Hs.1657 | NM_000125.1 |
| 205306_x_at | kynurenine 3-monooxygenase (kynurenine 3-hydroxylase) | Hs.107318 | AI074145 |
| 206165_s_at | chloride channel, calcium activated, family member 2 | Hs.241551 | NM_006536.2 |
| 206378_at | secretoglobin, family 2A, member 2 | Hs.46452 | NM_002411.1 |
| 207076_s_at | argininosuccinate synthetase | Hs.160786 | NM_000050.1 |
| 207131_x_at | gamma-glutamyltransferase 1 | Hs.284380 | NM_013430.1 |
| 208180_s_at | H4 histone family, member H | Hs.93758 | NM_003543.2 |
| 208614_s_at | filamin B, beta (actin binding protein 278) | Hs.81008 | M62994.1 |
| 209016_s_at | keratin 7 | Hs.23881 | BC002700.1 |
| 209603_at | GATA binding protein 3 | Hs.169946 | AI796169 |
| 210163_at | small inducible cytokine subfamily B (Cys-X-Cys), | Hs.103982 | AF030514.1 |
| | member 11 | | |
| 210519_s_at | diaphorase (NADHNADPH) (cytochrome b-5 reductase) | Hs.80706 | BC000906.1 |
| 210761_s_at | growth factor receptor-bound protein 7 | Hs.86859 | AB008790.1 |
| 211138_s_at | kynurenine 3-monooxygenase (kynurenine 3-hydroxylase) | Hs.107318 | BC005297.1 |
| 211430_s_at | immunoglobulin heavy constant gamma 3 (G3m marker) | Hs.300697 | M87789.1 |
| | gb: L06101.1 /DEF = Human IG VH-region gene, | | |
| | complete cds. /FEA = mRNA /GEN = | | |
| | IGH@ /PROD = immunoglobulin heavy | | |
| 211641_x_at | chain V-region /DB XREF = gi: 185526 | | L06101.1 |
| | gb: M85256.1 /DEF = Homo sapiens immunoglobulin | | |
| | kappa-chain VK-1 (IgK) mRNA, complete cds. /FEA = | | |
| | mRNA /GEN = IgK | | |
| 211645_x_at | /PROD = immunoglobulin kappa-chain VK-1 /DB_XREF = | | M85256.1 |
| | gi: 186008 gb: M18728.1 /DEF = Human nonspecific | | |
| | crossreacting antigen mRNA, complete cds. /FEA = | | |
| | mRNA /GEN = NCA; NCA; NCA | | |
| 211657_at | /PROD = non-specific cross reacting | | M18728.1 |
| | antigen /DB_XREF = gi: 189084 | | |
| 212218_s_at | F-box only protein 9 | Hs.11050 | NM_012347.1 |
| 212281_s_at | hypothetical protein | Hs.199695 | L19183.1 |
| 214451_at | transcription factor AP-2 beta (activating | Hs.33102 | NM_003221.1 |
| | enhancer binding protein 2 beta) | | |
| 214669_x_at | Homo sapiens isolate donor N clone N168K | Hs.306357 | BG485135 |
| | immunoglobulin kappa light chain variable region | | |
| | mRNA, partial cds | | |
| 215176_x_at | immunoglobulin kappa constant | Hs.156110 | AW404894 |
| 216557_x_at | Homo sapiens mRNA for single-chain antibody, | Hs.249245 | U92706 |
| | complete cds | | |
| 216836_s_at | v-erb-b2 erythroblastic leukemia viral oncogene | Hs.323910 | X03363.1 |
| | homolog 2, neuro/glioblastoma derived oncogene | | |
| | homolog (avian) | | |
| 217157_x_at | Homo sapiens isolate donor N clone N8K | Hs.247911 | AF103530.1 |
| | immunoglobulin kappa light chain variable region | | |
| | mRNA, partial cds | | |
| 217388_s_at | kynureninase (L-kynurenine hydrolase) | Hs.169139 | D55639.1 |
| 217480_x_at | Human kappa-immunoglobulin germline pseudogene | Hs.278448 | M20812 |
| | (cos118) variable region (subgroup V kappa I) | | |
| 219768_at | hypothetical protein FLJ22418 | Hs.36583 | NM_024626.1 |
| 220038_at | serum/glucocorticoid regulated kinase-like | Hs.279696 | NM_013257.1 |

Normal/Normal-like

| | | | |
|---|---|---|---|
| 201030_x_at | lactate dehydrogenase B | Hs.234489 | NM_002300.1 |
| 201792_at | AE binding protein 1 | Hs.118397 | NM_001129.2 |
| 201860_s_at | plasminogen activator, tissue | Hs.274404 | NM_000930.1 |
| 202037_s_at | secreted frizzled-related protein 1 | Hs.7306 | NM_003012.2 |
| 202218_s_at | fatty acid desaturase 2 | Hs.184641 | NM_004265.1 |
| 202662_s_at | inositol 1,4,5-triphosphate receptor, type 2 | Hs.238272 | NM_002223.1 |
| 202746_at | integral membrane protein 2A | Hs.17109 | AL021786 |
| 202887_s_at | HIF-1 responsive RTP801 | Hs.111244 | NM_019058.1 |
| 203058_s_at | 3′-phosphoadenosine 5′-phosphosulfate | Hs.274230 | AW299958 |
| | synthase 2 | | |
| 203213_at | cell division cycle 2, G1 to S and G2 to M | Hs.334562 | AL524035 |
| 203325_s_at | collagen, type V, alpha 1 | Hs.146428 | AI130969 |
| 203685_at | B-cell CLL/lymphoma 2 | Hs.79241 | NM_000633.1 |
| 203706_s_at | frizzled homolog 7 (Drosophila) | Hs.173859 | NM_003507.1 |
| 203755_at | BUB1 budding uninhibited by benzimidazoles 1 homolog | Hs.36708 | NM_001211.2 |
| | beta (yeast) | | |
| 203789_s_at | sema domain, immunoglobulin domain (Ig), short basic | Hs.171921 | NM_006379.1 |
| | domain, secreted, (semaphorin) 3C | | |
| 203878_s_at | matrix metalloproteinase 11 (stromelysin 3) | Hs.155324 | NM_005940.2 |
| 203915_at | monokine induced by gamma interferon | Hs.77367 | NM_002416.1 |
| 204033_at | thyroid hormone receptor interactor 13 | Hs.6566 | NM_004237.1 |
| 204602_at | dickkopf homolog 1 (Xenopus laevis) | Hs.40499 | NM_012242.1 |
| 204731_at | transforming growth factor, beta receptor III | Hs.342874 | NM_003243.1 |
| | (betaglycan, 300 kD) | | |
| 205034_at | cyclin E2 | Hs.30464 | NM_004702.1 |
| 205239_at | amphiregulin (schwannoma-derived growth factor) | Hs.270833 | NM_001657.1 |
| 207714_s_at | serine (or cysteine) proteinase inhibitor, clade H | Hs.241579 | NM_004353.1 |
| | (heat shock protein 47), member 1, (collagen | | |
| | binding protein 1) gb: NM_018407.1 /DEF = Homo | | |
| | sapiens putative integral membrane transporter | | |
| | (LC27), mRNA. /FEA = mRNA | | |
| 208029_s_at | /GEN = LC27 /PROD = putative integral | | NM_018407.1 |
| | membrane transporter /DB_XREF = gi: 8923827 | | |
| | clusterin (complement lysis inhibitor, SP-40, 40, | | |
| | sulfated glycoprotein 2, testosterone-repressed | | |
| | prostate message 2, | | |
| 208791_at | apolipoprotein J) clusterin (complement lysis | Hs.75106 | M25915.1 |
| | inhibitor, SP-40, 40, sulfated glycoprotein 2, | | |
| | testosterone-repressed prostate message 2, | | |
| 208792_s_at | apolipoprotein J) | Hs.75106 | M25915.1 |
| 209071_s_at | regulator of G-protein signalling 5 | Hs.24950 | AF159570.1 |
| 209218_at | squalene epoxidase | Hs.71465 | AF098865.1 |
| 209291_at | inhibitor of DNA binding 4, dominant negative | Hs.34853 | NM_001546.1 |
| | helix-loop-helix protein | | |
| 209292_at | Inhibitor of DNA binding 4, dominant negative | Hs.34853 | NM_001546.1 |
| | helix-loop-helix protein | | |
| 209465_x_at | pleiotrophin (heparin binding growth factor 8, neurite | Hs.44 | AL565812 |
| | growth-promoting factor 1) | | |
| 209687_at | stromal cell-derived factor 1 | Hs.237356 | U19495.1 |
| 210519_s_at | diaphorase (NADHNADPH) (cytochrome b-5 reductase) | Hs.80706 | BC000906.1 |
| | gb: M18728.1 /DEF = Human nonspecific | | |
| | crossreacting antigen mRNA, complete cds. /FEA = | | |
| | mRNA /GEN = NCA; | | |
| 211657_at | NCA; NCA /PROD = non-specific cross reacting | | M18728.1 |
| | antigen /DB_XREF = gi: 189084 | | |
| 211737_x_at | pleiotrophin (heparin binding growth factor 8, neurite | Hs.44 | BC005916.1 |
| | growth-promoting factor 1) | | |
| 212236_x_at | keratin 17 | Hs.2785 | Z19574 |
| 212254_s_at | bullous pemphigoid antigen 1 (230/240 kD) | Hs.198689 | BG253119 |
| 212592_at | Homo sapiens, done MGC: 24130 IMAGE: 4692359, mRNA, | Hs.76325 | AV733266 |
| | complete cds | | |
| 212730_at | KIAA0353 protein | Hs.10587 | AK026420.1 |
| 214290_s_at | H2A histone family, member O | Hs.795 | AA451996 |
| 216836_s_at | v-erb-b2 erythroblastic leukemia viral oncogene | Hs.323910 | X03363.1 |
| | homolog 2, neuro/glioblastoma derived oncogene | | |
| | homolog (avian) | | |
| 217428_s_at | collagen, type X, alpha 1 (Schmid metaphyseal | Hs.179729 | X98568 |
| | chondrodysplasia) | | |
| 218087_s_at | SH3-domain protein 5 (ponsin) | Hs.108924 | NM_015385.1 |
| 219115_s_at | interleukin 20 receptor, alpha | Hs.21814 | NM_014432.1 |
| 219197_s_at | CEGP1 protein | Hs.222399 | AI424243 |
| 219215_s_at | solute carrier family 39 (zinc transporter), | Hs.352415 | NM_017767.1 |
| | member 4 | | |
| 219304_s_at | spinal cord-derived growth factor-B | Hs.112885 | NM_025208.1 |
| 219768_at | hypothetical protein FLJ22418 | Hs.36563 | NM_024626.1 |
| 220038_at | serum/glucocorticoid regulated kinase-like | Hs.279696 | NM_013257.1 |
| 222155_s_at | hypothetical protein FLJ11856 | Hs.6459 | AK021918.1 |

TABLE 6b
2 Optimal Predictor Sets Using the GA/MLHD Algorithm

| | | | |
|---|---|---|---|
| Probe | Gene | Unigene | GeneBank |

Gene set 1

| | | | |
|---|---|---|---|
| 200926_at | ribosomal protein S23 | Hs.3463 | NM_001025.1 |
| 205225_at | estrogen receptor 1 | Hs.1657 | NM_000125.1 |
| 200670_at | X-box binding protein 1 | Hs.149923 | NM_005080.1 |
| 208248_{—} | amyloid beta (A4) | Hs.279518 | NM_001642.1 |
| x_at | precursor-like protein 2 | | |
| 209343_at | hypothetical protein | Hs.24391 | BC002449.1 |
| | FLJ13612 | | |
| 213399_{—} | ribophorin II | Hs.75722 | AI560720 |
| x_at | | | |
| 214938_{—} | high-mobility group | Hs.274472 | AF283771.2 |
| x_at | (nonhistone chromosomal) | | |
| | protein 1 | | |
| 207783_{—} | hypothetical protein | Hs.326456 | NM_017627.1 |
| x_at | FLJ20030 | | |
| 204533_at | small inducible cytokine | Hs.2248 | NM_001565.1 |
| | subfamily B (Cys-X-Cys), | | |
| | member 10 | | |
| 204798_at | v-myb myeloblastosis | Hs.1334 | NM_005375.1 |
| | viral oncogene homolog | | |
| | (avian) | | |
| 212790_{—} | ribosomal protein L13a | Hs.119122 | BF942308 |
| x_at | | | |
| 217276_{—} | serine hydrolase-like | Hs.301947 | AL590118.1 |
| x_at | | | |
| 213975_{—} | tudor repeat associator | Hs.283761 | AV711904 |
| s_at | with PCTAIRE 2 | | |
| 202428_{—} | diazepam binding | Hs.78888 | NM_020548.1 |
| x_at | inhibitor (GABA receptor | | |
| | modulator, | | |
| | acyl-Coenzyme A binding | | |
| | protein) | | |
| 200925_at | cytochrome c oxidase | Hs.180714 | NM_004373.1 |
| | subunit Via polypeptide 1 | | |

Gene set 2

| | | | |
|---|---|---|---|
| 221729_at | collagen, type V, alpha 2 | Hs.82985 | NM_000393.1 |
| 206461_{—} | metallothionein 1H | Hs.2667 | NM_005951.1 |
| x_at | | | |
| 205509_at | carboxypeptidase B1 | Hs.180884 | NM_001871.1 |
| | (tissue) | | |
| 212320_at | tubulin, beta polypeptide | Hs.179661 | BC001002.1 |
| 209043_at | 3′-phosphoadenosine | Hs.3833 | AF033026.1 |
| | 5′-phosphosulfate | | |
| | synthase 1 | | |
| 200032_{—} | ribosomal protein L9 | Hs.157850 | NM_000661.1 |
| s_at | | | |
| 202088_at | LIV-1 protein, estrogen | Hs.79136 | AI635449 |
| | regulated | | |
| 209604_{—} | GATA binding protein 3 | Hs.169946 | BC003070.1 |
| s_at | | | |
| 201892_{—} | IMP (inosine monophos- | Hs.75432 | NM_000884.1 |
| s_at | phate) dehydrogenase 2 | | |
| 211896_{—} | decorin | Hs.76152 | AF138302.1 |
| s_at | | | |
| 201952_at | activated leucocyte cell | Hs.10247 | NM_001627.1 |
| | adhesion molecule | | |
| 216836_{—} | v-erb-b2 erythroblastic | Hs.323910 | X03363.1 |
| s_at | leukemia viral oncogene | | |
| | homolog 2, neuro/glio- | | |
| | blastoma derived oncogene | | |
| | homolog (avian) | | |

TABLE 7
Up Regulated in luminal D

| Gene Name | Title | Unigene_Accession | Seq_Derived_From |
|---|---|---|---|
| 201422_at | interferon, gamma-inducible protein 30 | Hs.14623 | NM_006332.1 |
| 201577_at | non-metastatic cells 1, protein (NM23A) expressed in | Hs.118638 | NM_000269.1 |
| 201884_at | carcinoembryonic antigen-related cell adhesion molecule 5 | Hs.220529 | NM_004363.1 |
| 201946_s_at | chaperonin containing TCP1, subunit 2 (beta) | Hs.6456 | AL545982 |
| 202433_at | UDP-galactose transporter related | Hs.154073 | NM_005827.1 |
| 202779_s_at | ubiquitin carrier protein | Hs.174070 | NM_014501.1 |
| 203628_at | insulin-like growth factor 1 receptor | Hs.239176 | NM_000875.2 |
| 204566_at | protein phosphatase 1D magnesium-dependent, delta isoform | Hs.100980 | NM_003620.1 |
| 204868_at | immature colon carcinoma transcript 1 | Hs.9078 | NM_001545.1 |
| 211762_s_at | karyopherin alpha 2 (RAG cohort 1, importin alpha 1) | Hs.159557 | BC005978.1 |
| 211958_at | Homo sapiens, clone IMAGE: 4183312, mRNA, partial cds | Hs.180324 | L27560.1 |
| 211959_at | Homo sapiens, clone IMAGE: 4183312, mRNA, partial cds | Hs.180324 | L27560.1 |
| 217755_at | hematological and neurological expressed 1 | Hs.109706 | NM_016185.1 |
| 218585_s_at | RA-regulated nuclear matrix-associated protein | Hs.126774 | NM_016448.1 |
| 218732_at | CGI-147 protein | Hs.12677 | NM_016077.1 |
| 219493_at | hypothetical protein FLJ22009 | Hs.123253 | NM_024745.1 |
| 222039_at | hypothetical protein FLJ11029 | Hs.274448 | AA292789 |
| 222231_s_at | hypothetical protein PRO1855 | Hs.283558 | AK025328.1 |

Down Regulated in luminal D

| Gene Name | Title | Unigene_Accession [A] | Seq_Derived_From |
|---|---|---|---|
| 201667_at | gap junction protein, alpha 1, 43kD (connexin 43) | Hs.74471 | NM_000165.2 |
| 201939_at | serum-inducible kinase | Hs.3838 | NM_006622.1 |
| 202291_s_at | matrix Gla protein | Hs.365706 | NM_000900.1 |
| 203143_s_at | KIAA0040 gene product | Hs.158282 | T79953 |
| 203892_at | WAP four-disulfide core domain 2 | Hs.2719 | NM_006103.1 |
| 203917_at | coxsackie virus and adenovirus receptor | Hs.79187 | NM_001338.1 |
| 204942_s_at | aldehyde dehydrogenase 3 family, member B2 | Hs.87539 | NM_000695.2 |
| 205381_at | 37 kDa leucine-rich repeat (LRR) protein | Hs.155545 | NM_005824.1 |
| 205590_at | RAS guanyl releasing protein 1 (calcium and DAG-regulated) | Hs.182591 | NM_005739.2 |
| 208798_x_at | golgin-67 | Hs.182982 | AF204231.1 |
| 209189_at | v-fos FBJ murine osteosarcoma viral oncogene homolog | Hs.25647 | BC004490.1 |
| 212708_at | Homo sapiens mRNA; cDNA DKFZp586B1922 (from clone DKFZp586B1922) | Hs.184779 | AV721987 |
| 212927_at | KIAA0594 protein | Hs.103283 | AB011166.1 |
| 213089_at | ESTs, Highly similar to T17212 hypothetical protein DKFZp434P211.1 | Hs.352339 | AU158490 |
| | [H. sapiens] | | |
| 213605_s_at | Homo sapiens mRNA; cDNA DKFZp564F112 (from clone DKFZp564F112) | Hs.166361 | AL049987.1 |
| 214020_x_at | integrin, beta 5 | Hs.149846 | AI335208 |
| 214053_at | Homo sapiens clone 23736 mRNA sequence | Hs.7888 | AW772192 |
| 214218_s_at | Homo sapiens cDNA FLJ30298 fis, clone BRACE2003172 | Hs.351546 | AV699347 |
| 214657_s_at | multiple endocrine neoplasia I | Hs.240443 | AU134977 |
| 214705_at | PDZ domain protein (Drosophila inaD-like) | Hs.321197 | AJ001306.1 |
| 215071_s_at | H2A histone family, member L | HS.28777 | AL353759 |
| 215470_at | Human chromosome 5q13.1 clone 5G8 mRNA | Hs.14658 | U21915.1 |
| 217838_s_at | RNB6 | Hs.241471 | NM_016337.1 |
| 218312_s_at | hypothetical protein FLJ12895 | Hs.235390 | NM_023926.1 |
| 218330_s_at | retinoic acid inducible in neuroblastoma | Hs.23467 | NM_018162.1 |
| 218344_s_at | hypothetical protein FLJ10876 | Hs.94042 | NM_018254.1 |
| 218398_at | mitochondrial ribosomal protein S30 | Hs.28555 | NM_016640.1 |

1. A method of creating an expression profile characteristic of a breast tumor cell, said method comprising the steps of
(a) isolating expression products from said breast tumor cell and a normal breast cell;
(b) contacting said expression products for both the tumor and normal breast cell with a plurality of binding members capable of specifically binding to expression products of at least 10 genes selected from Table 2; so as to create an expression profile of those genes for both the tumor cell and the normal cell;
(c) comparing the expression profile of the tumor cell and the normal cell; and
(d) determining an expression profile characteristic of a breast tumor cell.

2-66. (canceled)

67. The method as set forth in claim 1 wherein the binding members are capable of specifically and independently binding to each of the genes provided in Table 2.

68. The method as set forth in claim 67 wherein the expression product is a polypeptide.

69. The method as set forth in claim 68 wherein the binding members are antibody binding domains.

70. The method as set forth in claim 67 wherein the expression product is mRNA or cDNA.

71. The method as set forth in claim 70 wherein the binding members are nucleic acid probes.

72. The method as set forth in claim 71 wherein the binding members are labelled.

73. The method as set forth in claim 70 wherein the expression products are labelled.

74. A method of creating an expression profile characteristic of a breast tumor cell, said method comprising the steps of
(a) isolating expression products from a breast tumor cell, contacting said expression products with a plurality of binding members capable of specifically and independently binding to expression products of at least 10 genes selected from Table 2; so as to create a first expression profile of a tumor cell;
(b) isolating expression products from a normal breast cell; contacting said expression products with the plurality of binding members as used in step (a), so as to create a comparable second expression profile of a normal breast cell; and
(c) comparing the first and second expression profiles to determine an expression profile characteristic of a breast tumor cell.

75. The method as set forth in claim 74 wherein the binding members are capable of specifically and independently binding to each of the genes provided in Table 2.

76. The method as set forth in claim 75 wherein the expression product is a polypeptide.

77. The method as set forth in claim 76 wherein the binding members are antibody binding domains.

78. The method as set forth in claim 75 wherein the expression product is mRNA or cDNA.

79. The method as set forth in claim 78 wherein the binding members are nucleic acid probes.

80. The method as set forth in claim 79 wherein the binding members are labelled.

81. The method as set forth in claim 78 wherein the expression products are labelled.

82. A method of creating a nucleic acid expression profile characteristic of a breast tumor cell, said method comprising the steps of
(a) isolating expression products from a first breast tumor cell, contacting said expression products with a plurality of binding members capable of specifically and independently binding to expression products of at least 10 genes selected from Table 2, so as to create a first expression profile;
(b) repeating step (a) with expression products from at least a second breast tumor cell so as to create at least a second expression profile;
(c) comparing the at least first and second expression profiles to create a standard nucleic acid expression profile characteristic of a breast tumor cell.

83. The method as set forth in claim 82 wherein the isolated expression products are contacted with a plurality of binding members capable of specifically and independently binding to expression products of each of the genes provided in Table 2.

84. The method as set forth in claim 83 wherein the expression product is a polypeptide.

85. The method as set forth in claim 84 wherein the binding members are antibody binding domains.

86. The method as set forth in claim 83 wherein the expression product is mRNA or cDNA.

87. The method as set forth in claim 86 wherein the binding members are nucleic acid probes.

88. The method as set forth in claim 87 wherein the binding members are labelled.

89. The method as set forth in claim 86 wherein the expression products are labelled.

90. A method for determining the presence or risk of breast cancer in an individual, said method comprising
(a) obtaining expression products from a breast tissue cell obtained from an individual suspected of having or at risk from having breast cancer;
(b) contacting said expression products with binding members capable of specifically and independently binding to expression products corresponding to at least 10 of the genes identified in Table 2; and
(c) determining the presence or risk of breast cancer in said individual based on the binding of the expression products from said breast

91. The method as set forth in claim 90 wherein the expression products are contacted with binding members are capable of specifically and independently binding to expression products corresponding to each of the genes identified in Table 2.

92. The method as set forth in claim 91 wherein the determination of the presence or risk of breast cancer in said individual is carried out by comparing the binding of the expression products from the breast tissue cell under test with an expression profile characteristic of breast tumor cell.

93. The method as set forth in claim 92 wherein the individual is of Asian descent.

94. A method of creating a nucleic acid expression profile characteristic of a breast tumor cell, said method comprising the steps of
(a) isolating expression products from said breast tumor cell and a normal breast cell;
(b) contacting said expression products for both the tumor and normal breast cell with a plurality of binding members capable of specifically binding to expression products of at least 10 genes selected from Table 4a; so as to create an expression profile of those genes for both the tumor cell and the normal cell;
(c) comparing the expression profile of the tumor cell and the normal cell; and
(d) determining a nucleic acid expression profile characteristic of breast tumor cell.

95. The method as set forth in claim 94 wherein the isolated expression products are contacted with a plurality of binding members capable of specifically and independently binding to expression products of at least 10 genes selected from Table 4b.

96. The method as set forth in claim 95 wherein the binding expression product is mRNA or cDNA.

97. The method as set forth in claim 95 wherein the binding members are nucleic acid probes.

98. The method as set forth in claim 95 wherein the expression product is a polypeptide.

99. The method as set forth in claim 98 wherein the binding members are antibody binding domains.

100. The method as set forth in claim 99 wherein the binding members are labelled.

101. The method as set forth in claim 99 wherein the expression products are labelled.

102. A method of creating a nucleic acid expression profile characteristic of a breast tumor cell, said method comprising the steps of
(a) isolating expression products from a breast tumor cell; contacting said expression products with a plurality of binding members capable of specifically and independently binding to expression products of at least 10 genes selected from Table 4a; so as to create a first expression profile of a tumor cell;
(b) isolating expression products from a normal breast cell; contacting said expression products with the plurality of binding members as used in step (a); so as to create a comparable second expression profile of a normal breast cell;
(c) comparing the first and second expression profiles to determine an expression profile characteristic of a breast tumor cell.

103. The method as set forth in claim 102 wherein the isolated expression products are contacted with a plurality of binding members capable of specifically and independently binding to expression products of at least 10 genes selected from Table 4b.

104. The method as set forth in claim 102 wherein the isolated expression products are contacted with a plurality of binding members capable of specifically and independently binding to expression products of at least twenty genes selected from Table 4a.

105. The method as set forth in claim 102 wherein the binding expression product is mRNA or cDNA.

106. The method as set forth in claim 102 wherein the binding members are nucleic acid probes.

107. The method as set forth in claim 102 wherein the expression product is a polypeptide.

108. The method as set forth in claim 107 wherein the binding members are antibody binding domains.

109. The method as set forth in claim 107 wherein the binding members are labelled.

110. The method as set forth in claim 107 wherein the expression products are labelled.

111. A method for determining the presence or risk of breast cancer in an individual, said method comprising
(a) obtaining expression products from a breast tissue cell obtained from an individual suspected of having or at risk from having breast cancer;
(b) contacting said expression products with binding members capable of binding to expression products corresponding to at least 10 genes identified in Table 4a; and
(c) determining the presence or risk of breast cancer in said individual based on the binding of the expression products from said breast tissue cell to one or more of the binding members.

112. The method as set forth in claim 111 wherein the determination of the presence or risk of breast cancer is computed using an algorithm which distinguishes a tumor cell from normal cell by their respective expression profiles.

113. The method as set forth in claim 111 wherein the determination of the presence or risk of breast cancer in said individual is carried out by comparing the binding of the expression products from the breast tissue cell under test with an expression profile characteristic of breast tumor cell.

114. The method as set forth in claim 111 wherein the expression products are contacted with a plurality of binding members are capable of binding to expression products of at least twenty genes selected from Table 4a.

115. The method as set forth in claim 114 wherein the determination of the presence or risk of breast cancer is computed using an algorithm which distinguishes a tumor cell from normal cell by their respective expression profiles.

116. The method as set forth in claim 114 wherein the determination of the presence or risk of breast cancer in said individual is carried out by comparing the binding of the expression products from the breast tissue cell under test with an expression profile characteristic of breast tumor cell.

117. The method as set forth in claim 111 wherein the expression products are contacted with a plurality of binding members are capable of binding to expression products of at least 10 genes identified in Table 4b.

118. The method as set forth in claim 117 wherein the determination of the presence or risk of breast cancer is computed using an algorithm which distinguishes a tumor cell from normal cell by their respective expression profiles.

119. The method as set forth in claim 117 wherein the determination of the presence or risk of breast cancer in said individual is carried out by comparing the binding of the expression products from the breast tissue cell under test with an expression profile characteristic of breast tumor cell.

120. A method of obtaining a plurality of gene expression profiles in order to determine a standard expression profile characteristic of presence and/or type of breast cancer, said method comprising
a) obtaining cells from a plurality of breast tumor sample;
b) disrupting said cells to expose gene expression products;
c) contacting said gene expression products with a plurality of binding members specific for expression products of at least 10 genes selected from Table 2; and
d) determining a gene expression profile characteristic of the presence and/or type of breast cancer based on the binding of said expression products to said binding members for each of said plurality of breast tumor samples.

121. The method as set forth in claim 120 further comprising the step of producing a database containing a plurality of expression profiles obtained from said plurality of breast tumor samples.

122. The method as set forth in claim 120 further comprising the step of determining the statistical variation between the plurality of expression profiles.

123. A method of obtaining a plurality of gene expression profiles in order to determine a standard expression profile characteristic of presence and/or type of breast cancer, said method comprising
a) obtaining cells from a plurality of breast tumor sample;
b) disrupting said cells to expose gene expression products;
c) contacting said gene expression products with a plurality of binding members specific for expression products of at least 10 genes selected from Table 4a; and
d) determining a gene expression profile characteristic of the presence and/or type of breast cancer based on the binding of said expression products to said binding members for each of said plurality of breast tumor samples.

124. The method as set forth in claim 123 further comprising the step of producing a database containing a plurality of expression profiles obtained from said plurality of breast tumor samples.

125. The method as set forth in claim 123 further comprising the step of determining the statistical variation between the plurality of expression profiles.

126. A database comprising expression profiles characteristic of breast cancer or type of breast cancer produced by the method as set forth in claim 125.

127. The database as set forth in claim 126 wherein the expression profiles are nucleic acid expression profiles.

128. The database as set forth in claim 126 wherein the expression profiles are protein expression profiles.

129. A method of obtaining a plurality of gene expression profiles in order to determine a standard expression profile characteristic of presence and/or type of breast cancer, said method comprising
a) obtaining cells from a plurality of breast tumor sample;
b) disrupting said cells to expose gene expression products;
c) contacting said gene expression products with a plurality of binding members specific for expression products of at least 10 genes selected from Table 4b; and
d) determining a gene expression profile characteristic of the presence and/or type of breast cancer based on the binding of said expression products to said binding members for each of said plurality of breast tumor samples.

130. The method as set forth in claim 129 further comprising the step of producing a database containing a plurality of expression profiles obtained from said plurality of breast tumor samples.

131. The method as set forth in claim 129 further comprising the step of determining the statistical variation between the plurality of expression profiles.

132. A database comprising expression profiles characteristic of breast cancer or type of breast cancer produced by the method as set forth in claim 131.

133. The database as set forth in claim 132 wherein the expression profiles are nucleic acid expression profiles.

134. The database as set forth in claim 132 wherein the expression profiles are protein expression profiles.

135. A method of obtaining a plurality of gene expression profiles in order to determine a standard expression profile characteristic of presence and/or type of breast cancer, said method comprising
a) obtaining cells from a plurality of breast tumor sample;
b) disrupting said cells to expose gene expression products;
c) contacting said gene expression products with a plurality of binding members specific for expression products of at least 10 genes selected from Table 5; and
d) determining a gene expression profile characteristic of the presence and/or type of breast cancer based on the binding of said expression products to said binding members for each of said plurality of breast tumor samples.

136. The method as set forth in claim 135 further comprising the step of producing a database containing a plurality of expression profiles obtained from said plurality of breast tumor samples.

137. The method as set forth in claim 135 further comprising the step of determining the statistical variation between the plurality of expression profiles.

138. A database comprising expression profiles characteristic of breast cancer or type of breast cancer produced by the method as set forth in claim 137.

139. The database as set forth in claim 138 wherein the expression profiles are nucleic acid expression profiles.

140. The database as set forth in claim 138 wherein the expression profiles are protein expression profiles.

141. A method of obtaining a plurality of gene expression profiles in order to determine a standard expression profile characteristic of presence and/or type of breast cancer, said method comprising
a) obtaining cells from a plurality of breast tumor sample;
b) disrupting said cells to expose gene expression products;
c) contacting said gene expression products with a plurality of binding members specific for expression products of at least 10 genes selected from Table 6a; and
d) determining a gene expression profile characteristic of the presence and/or type of breast cancer based on the binding of said expression products to said binding members for each of said plurality of breast tumor samples.

142. The method as set forth in claim 141 further comprising the step of producing a database containing a plurality of expression profiles obtained from said plurality of breast tumor samples.

143. The method as set forth in claim 141 further comprising the step of determining the statistical variation between the plurality of expression profiles.

144. A database comprising expression profiles characteristic of breast cancer or type of breast cancer produced by the method as set forth in claim 143.

145. The database as set forth in claim 144 wherein the expression profiles are nucleic acid expression profiles.

146. The database as set forth in claim 144 wherein the expression profiles are protein expression profiles.

147. A method of obtaining a plurality of gene expression profiles in order to determine a standard expression profile characteristic of presence and/or type of breast cancer, said method comprising
a) obtaining cells from a plurality of breast tumor sample;
b) disrupting said cells to expose gene expression products;
c) contacting said gene expression products with a plurality of binding members specific for expression products of at least 10 genes selected from Table 7; and
d) determining a gene expression profile characteristic of the presence and/or type of breast cancer based on the binding of said expression products to said binding members for each of said plurality of breast tumor samples.

148. The method as set forth in claim 147 further comprising the step of producing a database containing a plurality of expression profiles obtained from said plurality of breast tumor samples.

149. The method as set forth in claim 147 further comprising the step of determining the statistical variation between the plurality of expression profiles.

150. A database comprising expression profiles characteristic of breast cancer or type of breast cancer produced by the method as set forth in claim 149.

151. The database as set forth in claim 150 wherein the expression profiles are nucleic acid expression profiles.

152. The database as set forth in claim 150 wherein the expression profiles are protein expression profiles.

153. A method of obtaining a plurality of gene expression profiles in order to determine a standard expression profile characteristic of presence and/or type of breast cancer, said method comprising
a) obtaining cells from a plurality of breast tumor sample;
b) disrupting said cells to expose gene expression products;
c) contacting said gene expression products with a plurality of binding members capable of specifically and independently binding to expression products of the genes identified in Table 6b;
d) determining a gene expression profile characteristic of the presence and/or type of breast cancer based on the binding of said expression products to said binding members for each of said plurality of breast tumor samples.

154. The method as set forth in claim 153 further comprising the step of producing a database containing a plurality of expression profiles obtained from said plurality of breast tumor samples.

155. The method as set forth in claim 153 further comprising the step of determining the statistical variation between the plurality of expression profiles.

156. A database comprising expression profiles characteristic of breast cancer or type of breast cancer produced by the method as set forth in claim 155.

157. The database as set forth in claim 156 wherein the expression profiles are nucleic acid expression profiles.

158. The database as set forth in claim 156 wherein the expression profiles are protein expression profiles.

159. A method for classifying a breast tumor cell on the basis of Estrogen receptor (ER) status, said method comprising
(a) obtaining expression products from a breast tumor cell;
(b) contacting said expression products with binding members capable of binding to expression products corresponding to the genes identified in Table 5a; and
(c) classifying the breast tumor on the basis of ER status based on the binding of the expression products from said breast tumor cell to one or more of the binding members.

160. A method for classifying a breast tumor cell on the basis of ERBB2 status, said method comprising
(a) obtaining expression products from a breast tumor cell;
(b) contacting said expression products with binding members capable of binding to expression products corresponding to the genes identified in Table 5b; and
(c) classifying the breast tumor on the basis of ERBB2 status based on the binding of the expression products from said breast tumor cell to one or more of the binding members.

161. A method for classifying a breast tumor cell on the basis of its molecular subtype, said method comprising
(a) obtaining expression products from a breast tumor cell;
(b) contacting said expression products with binding members capable of binding to expression products corresponding to at least 10 genes identified in Table 6a; and
(c) classifying the tumor cell with regard to its molecular subtype based on the binding profile of the expression products from the tumor cell and the binding members.

162. The method as set forth in claim 161 wherein the binding members are capable of specifically and independently binding to at least twenty genes identified in Table 6a.

163. The method as set forth in claim 162 wherein the molecular subtypes are selected from Luminal, ERBB2, Basal, ER-type II and normal/normal-like.

164. The method as set forth in claim 161 wherein the binding members are capable of specifically and independently binding to at least the genes identified in Table 6b.

165. The method as set forth in claim 164 wherein the molecular subtypes are selected from Luminal, ERBB2, Basal, ER-type II and normal/normal-like.

166. A method for classifying a breast tumor cell on the basis of its Luminal sub-class, said method comprising
(a) obtaining expression products from a breast tumor cell;
(b) contacting said expression products with binding members capable of binding to expression products corresponding to at least 10 genes identified in Table 7; and
(c) classifying the tumor cell with regard to its Luminal sub-class based on the binding profile of the expression products from the tumor cell and the binding members.

167. The method as set forth in claim 166 wherein said tumor cell has been previously classified as a Luminal molecular subtype.

168. The method as set forth in claim 167 wherein the Luminal sub-class is Luminal D or Luminal A.

169. A diagnostic tool comprising a plurality of binding members capable of specifically and independently binding to expression products of at least 10 genes selected from Table 4a, said plurality of binding members being fixed to a solid support.

170. The diagnostic tool as set forth in claim 169 wherein said binding members are cDNA or oligonucleotides.

171. A diagnostic tool comprising a plurality of binding members capable of specifically and independently binding to expression products of at least 10 genes selected from Table 4b, said plurality of binding members being fixed to a solid support.

172. The diagnostic tool as set forth in claim 171 wherein said binding members are cDNA or oligonucleotides.

173. A diagnostic tool comprising a plurality of binding members capable of specifically and independently binding to expression products of at least 10 genes selected from Table 5a, said plurality of binding members being fixed to a solid support.

174. The diagnostic tool as set forth in claim 173 wherein said binding members are cDNA or oligonucleotides.

175. A diagnostic tool comprising a plurality of binding members capable of specifically and independently binding to expression products of at least 10 genes selected from Table 5b, said plurality of binding members being fixed to a solid support.

176. The diagnostic tool as set forth in claim 175 wherein said binding members are cDNA or oligonucleotides.

177. A diagnostic tool comprising a plurality of binding members capable of specifically and independently binding to expression products of at least 10 genes selected from Table 6a, said plurality of binding members being fixed to a solid support.

178. The diagnostic tool as set forth in claim 177 wherein said binding members are cDNA or oligonucleotides.

179. A diagnostic tool comprising a plurality of binding members capable of specifically and independently binding to expression products of at least 10 genes selected from Table 7, said plurality of binding members being fixed to a solid support.

180. The diagnostic tool as set forth in claim 179 wherein said binding members are cDNA or oligonucleotides.

181. A diagnostic tool comprising a plurality of binding members capable of specifically and independently binding to expression products of the genes identified in Table 6b, said plurality of binding members being fixed to a solid support.

182. The diagnostic tool as set forth in claim 181 wherein said binding members are cDNA or oligonucleotides.
